SEQUENCE LISTING (3 sequences)

SEQ ID NO: 1 - RNA, 88 nt, Homo sapiens
  source 1..88: mol_type = other RNA
caccttgtcctcacggtccagttttcccaggaatcccttagatgctaagatggggattcctggaaatactgttcttgaggtcatggtt

SEQ ID NO: 2 - RNA, 110 nt, Homo sapiens
  source 1..110: mol_type = other RNA
agaagggctatcaggccagccttcagaggactccaaggaacattcaacgctgtcggtgagtttgggatttgaaaaaaccactgaccgttgactgtaccttggggtcctta

SEQ ID NO: 3 - DNA, 106 bp, Homo sapiens
  source 1..106: mol_type = genomic DNA
gcgcagcgccctgtctcccagcctgaggtgcagtgctgcatctctggtcagttgggagtctgagatgaagcactgtagctcaggaagagagaagttgttctgcagc

(19) United States
(12) Patent Application Publication
(10) Pub. No.: US 2026/0335366 A1
(43) Pub. Date: Oct. 29, 2026

(54) METHODS FOR TREATING MUSCLE WASTING DISEASES USING MBV

(71) Applicant: University of Pittsburgh - Of the Commonwealth System of Higher Education, Pittsburgh, PA (US)

(72) Inventors: Stephen Francis Badylak, West Lafayette, IN (US); George S. Hussey, Cranberry Township, PA (US); William Anthony D'Angelo, Pittsburgh, PA (US); Héctor Capella Monsonís, Pittsburgh, PA (US)

(73) Assignee: University of Pittsburgh - Of the Commonwealth System of Higher Education, Pittsburgh, PA (US)

(21) Appl. No.: 19/484,333

(22) PCT Filed: May 16, 2024
(86) PCT No.: PCT/US2024/029787

§ 371 Date: Nov. 13, 2025

Related U.S. Application Data
(60) Provisional application No. 63/502,751, filed on May 17, 2023.

(51) Int. Cl. A61K 35/22; A61K 35/15; A61P 21/00; C12N 5/071; C12N 5/0786

(52) U.S. Cl. CPC A61K 35/22; A61K 35/15; A61P 21/00; C12N 5/0645; C12N 5/0685

(57) ABSTRACT

Disclosed are methods for treating muscle wasting diseases, such as spinal muscular atrophy, or muscular dystrophies, using matrix bound vesicles (MBV). Compositions for use in treating a muscle wasting disease are also disclosed.

### CROSS REFERENCE TO RELATED APPLICATIONS

This claims the benefit of U.S. Provisional Patent Application No. 63/502,751, filed May 17, 2023, which is incorporated herein by reference in its entirety.

### FIELD OF THE DISCLOSURE

This disclosure is related to the field of treatment of muscle wasting conditions, specifically to the use of extracellular matrix (ECM) bound nanovesicles (MBV) to treat muscle wasting conditions, such as a muscular dystrophy or spinal muscle atrophy.

### SEQUENCE LISTING

The contents of the sequence listing (sequence listing.xml, Size: 3,760 bytes, Date of Creation: May 1, 2024) is herein incorporated by reference in its entirety.

### BACKGROUND

The loss of muscle strength and tissue deterioration affects numerous individuals. In particular, atrophy involves a wasting away or deterioration of an organ, tissue, or body part typically caused by disease, injury, or lack of use. Many different types of atrophy are known. The primary diseases involve muscle atrophy, which is the wasting or loss of muscle tissue; multiple-system atrophy, which is a degenerative neurological disorder associated with the degeneration of nerve cells in the brain; and spinal muscular atrophy, which affects the spinal cord and nerves. Typically diseases that cause or result in atrophy affect one or more muscles within the human body. Similarly, dystrophic disorders also are typically caused by disease and have substantially the same effect as atrophy on muscle.

The muscular dystrophies are a group of diverse, heritable neuromuscular disorders which represent a group of devastating neuromuscular diseases characterized by primary or secondary skeletal muscle involvement. Duchenne muscular dystrophy (DMD) is an X-chromosome-linked disease and the most common form of muscular dystrophy. DMD affects 1 in 3500 live male births with patients suffering from chronic muscle degeneration and weakness. Clinical symptoms are first detected between the ages of 2 and 5 years and, by the time the patient is in their teens, the ability for independent ambulation is lost. Death typically occurs in the patient before they are 30 years old due to cardiopulmonary failure. A need remains for methods of treating muscular dystrophy in a subject.

Facioscapulohumeral muscular dystrophy (FSHD) is a genetic muscle disorder resulting in primarily the progressive degeneration of the muscles of the face, shoulder blades, and upper arms, although weakness in other muscles also occurs. FSHD is the third most common type of muscular dystrophy with an estimated prevalence of about 3 cases per 100,000 individuals. In most individuals diagnosed with FSHD, symptoms begin before age 20. FSHD is caused by a mutation that leads to inappropriate expression of the double homeobox protein 4 gene (DUX4). There is currently no pharmaceutical treatment for FSHD, and so patients must manage with surgical and mechanical assistance such as by surgical procedures to stabilize the shoulder blades, use of orthoses such as back supports, girdles, and braces, as well as through low-intensity exercise. Accordingly, new therapies targeting muscle degeneration are needed to improve the quality of life of these subjects.

Spinal muscular atrophy (SMA) is a neurodegenerative disease triggered by a genetic mutation in the survival motoneuron 1 (SMN1) gene. Affected motoneurons in SMA patients are less-capable of producing sustained firing and can degrade over time, leading to motoneuron (MN) death. SMA severity ranges from respiratory failure in the neonatal period (type 1-2) to mild muscle weakness noticed in adulthood (type 4). New genetic therapies have had success preventing the necessity of permanent breathing support and the death of neonatal patients (type 1-2). However, these genetic therapies have been unable to help type 3-4 patients with severe locomotion deficits in adulthood. Moreover, type 1-2 patients treated with genetic therapies can develop severe locomotion deficits. Thus, new therapies targeting motor impairments in SMA patients and improving the effectiveness of current therapies are needed to improve the quality of life of these subjects.

### SUMMARY OF THE DISCLOSURE

Methods are disclosed for treating a subject with a muscle wasting condition. These methods include administering to the subject a composition comprising an effective amount of: a) exogenous matrix bound nanovesicles (MBV) derived from extracellular matrix, wherein the MBV do not express CD63 and CD81 or are CD63^{lo}CD81^{lo} and wherein the MBV do not contain alkaline phosphatase; b) myeloid progenitor cells or myeloid-derived cells treated with the exogenous MBV; and/or c) conditioned media or a fraction thereof obtained from a culture of macrophages and cultured in the presence of the exogenous MBV.

Methods are also disclosed for promoting muscle regeneration or repair in a subject in need thereof. These methods include administering to the subject a composition comprising an effective amount of exogenous matrix bound nanovesicles (MBV) derived from extracellular matrix, wherein the MBV do not express CD63 and CD81 or are CD63^{lo}CD81^{lo} and wherein the MBV do not contain alkaline phosphatase, thereby to promote muscle regeneration in the subject.

The foregoing and other features and advantages of the invention will become more apparent from the following detailed description of several aspects which proceeds with reference to the accompanying figures.

### BRIEF DESCRIPTION OF THE FIGURES

FIG. 1 is a schematic drawing showing the procedures used to determine the direct (left side) and indirect effects (right side) of MBV administration on the transcriptome of FSHD myoblasts as described in Examples 1-4.

FIG. 2. Metabolic activity (%) of healthy and FSHD2 myoblasts from patients treated with different doses of MBV for 24 hours.

FIG. 3. The effect of a macrophage-derived secretome (MBV, LPS (M1) and IL4 (M2)) on the metabolic activity (%) of HMEC-1 cells after 24 h (left) and 72 h (right).

FIG. 4. Results of a scratch assay showing % area migration of HMEC-1 cells after treatment with M1, M2 and M-MBV macrophage-derived secretome.

FIG. 5. The effect of a macrophage-derived secretome (MBV, LPS (M1) and IL4 (M2)) on the metabolic activity (%) of N1E-155 cells after 24 h (left) and 72 h (right).

FIGS. 6A-6B. The effect of a macrophage-derived secretome (MBV, LPS (M1) and IL4 (M2)) on N1E-155 cell differentiation after 24 and 72 hours measured by morphologic features of roundness (FIG. 5A top panel), eccentricity ratio (FIG. 5A bottom panel) and Feret diameter (FIG. 5B).

FIG. 7. Classification of bone marrow cells uptaking MBV after 3 and 24 h.

FIG. 8. Shows the distribution of accessible sequences in different treatments in vitro and in vivo from an ATAC-sequence analysis.

FIG. 9. Shows the distribution of statistically significant regulated genes at an epigenetic level in different cells treated in vitro. Dots are shown on the right side of each graph (corresponding to the larger circle in the Venn diagram at bottom of figure) and dots are on the left side of each graph, corresponding to the smaller circle in the Venn diagram. Dots in the middle of each graph correspond to the genes in the overlapping area of the Venn diagram; the middle section represents the overlapping portion of the Venn diagram. Similarly, in the Venn diagram, the large circle is the dots on the right side and the small circle is the dots on the left side.

FIGS. 10A-10B. Schematic experimental design of MBV accumulation in bone marrow cells (FIG. 10A). Bone marrow and blood fluorescence imaging (FIG. 10B) after 24 h of intraperitoneal injection of different MBV-tagged concentrations and their correspondent readings (FIG. 10A). This accumulation was associated with a change in response to M1 challenging (FIG. 10B).

FIG. 11. Schematic diagram of study in mouse models of Facioscapulohumeral Muscular Dystrophy (FSHD). Top: DUX4 expression was induced by feeding four-week-old female iDUX4 pA-HSA mice dox chow on Day 0 and maintained for the duration of the study to simulate a “moderate” Dux4 environment (iDUX4 pA-HSA mouse model). Bottom: DUX4 expression was induced by TMX injection on Day 0 in 14-week old males to simulate the FSHD phenotype in adult. A second arm of the study evaluated the effect of MBV treatment on the accumulative phenotype caused by low chronic DUX4 expression in FSHD aged mouse model (no TMX induction, 7-month-old mice; FLExDUX4(+TMX) mouse model).

FIG. 12 is a bar graph showing the FSHD severity score in normal (control) mice, MBV-treated FSHD mice, and saline-treated FSHD mice as described in Example 8. A score of zero (0) was given to tissue sections showing no injury (i.e., normal skeletal muscle tissue). A score of 1 showed mild injury. A score of 2 showed moderate injury with increased inflammatory cell presence around dead or dying muscle fibers. A score of 3 showed severe, diffuse injury with extensive inflammatory cell presence, activated satellite cells with the scattered presence of myoblasts, and multifocal tissue necrosis.

FIG. 13 shows photographs of exemplary muscle tissue samples from control (PBS treated; left) and MBV treated (right) FSHD mice in the FLExDUX4(+TMX) model. Hematoxylin and eosin stain at approximately 400× magnification.

FIG. 14 shows photographs of exemplary muscle tissue samples from control (PBS treated; left) and MBV treated (right) SMA mice. Hematoxylin and eosin stain at approximately 400× magnification. These images show longitudinal sections of skeletal muscle. The control treated animals show severe muscle atrophy, separation of muscle fibers, increased inflammatory cell presence between fibers, and areas of tissue necrosis. The MBV treated animals also show muscle atrophy but less that the PBS treated controls and less inflammation and less severe muscle damage.

FIG. 15 is a schematic diagram of the study in a mouse model of Spinal Muscular Atrophy (SMA). Mice received an intramuscular injection of MBV on day 3 and were sacrificed on Day 7.

FIGS. 16A-16B provide a comparison of surface markers for exosomes, bone microvesicles (MV) and MBV. The figure shows the results of EXO-CHECK™ Exosome Antibody Arrays (System Biosciences) comparing levels of the various markers noted in murine exosomes, murine bone matrix vesicles (bone MV), and murine matrix bound nanovesicles (MBV). FIG. 16A provides digital images of the arrays and FIG. 16B is a graph showing the relative expression of each of the noted markers in the exosomes versus bone MV versus MBV. The data show that MBV are different from exosomes, bone microvesicles (MV) based on the profile of surface markers. The MBV do not express or have low expression of CD63, EpCAM, ANXA5, TSG101, GM130, FLOT1, ICAM1, ALIX, and CD81, as compared to Bone MV or exosome levels of these markers as shown in the bar graphs in the lower panel.

FIG. 17 is a Western blot showing that bone MV markers Annexin V and Tissue Non-specific Alkaline Phosphatase (TNAP) are expressed by bone MV. Lysate prepared from 1711A Cells was used as a positive control. The results of this experiment show that matrix bound nanovesicles (MBV) are devoid of any expression of both markers of bone microvesicles, TNAP and Annexin V. Plasma exosomes do express Annexin V, but do not express TNAP. These results clearly distinguish MBV from both exosomes and bone microvesicles. Notably, the MBV used were isolated from muscle tissue.

FIG. 18 is a bar graph showing the different effects of macrophage activated-gene expression on exosomes, MV and MBV. MBV have a differential immunomodulatory effect, namely they increase M2 macrophages, when compared to exosomes or bone MV which do not have this effect. Bone Marrow-Derived Macrophages (BMDM) harvested from mice were untreated (M0) or treated with the following test articles for 24 hours: IFNγ+LPS to induce an M1 phenotype (M1), IL-4 to induce an M2-like phenotype (M2), Exosomes derived from plasma, bone MV derived from 17A cells, or MBV isolated from muscle. After treatment, the fold change in the expression of the indicated genes (Arg, CD206, Fixx, IL-6. INOS, and TNF) was evaluated by qPCR. The pro-inflammatory markers IL-6 and TNF-α by MBV were downregulated and are clearly distinguished from the downregulation of the same two inflammatory mediators by exosomes and bone MV. MBV had a potent anti-inflammatory effect; whereas exosomes and bone MV did not have this effect.

FIG. 19 is a schematic of the D2.mdx mouse study. D2.mdx mice (6-8 weeks old) were randomly assigned into groups 1) saline treatment, n=6 and 2) MBV treatment n=6. 100 μl containing 4.2×10\hat{ }9 of MBV were administered via intraperitoneal injection on days 1, 3, and 5, and then once weekly thereafter. Body weight was recorded weekly. Animals were sacrificed at week 8 and tissue was explanted, weighed and processed for histological examination.

FIG. 20 provides graphs showing body weight and organ weights from the D2.mdx mouse study. Animals (n=6 per group) were weighed at 1, 3, 5, 7, and 8 weeks. The data show a statistically significant difference in body weight between MBV treated and control animals. At 8 weeks, animals were sacrificed and the gastrocnemius (GC), tibialis anterior (TA), extensor digitorum longus (EDL), soleus (SOLL, Liver and Spleen were explanted and weighted. Results showed that the gastrocnemius (GC) showed significant differences between groups. Other muscles tested demonstrated a trend toward higher weights in the MBV treated group versus the control group.

FIG. 21 shows results from muscle function tests. Functional analysis was performed by measuring isometric torque production of the gastrocnemius at weeks 1 and 7. Results showed a statistically significant increase in torque production in animals treated with MBV compared to Saline Control.

FIG. 22 are representative digital images of muscle architecture (assessed by H&E), and fibrosis (assessed by Masson's trichrome). The saline treated group showed widespread myodegeneration, inflammation, thick bands of dense fibrous tissue that replaced lost muscle tissue, and a modest attempt at muscle cell regeneration. In contrast, the MBV-treated group showed much less severe muscle degeneration, sparse and widely scattered areas of myodegeneration, a small number of scattered mononuclear inflammatory cells, and a robust muscle regenerative response as evidenced by the satellite cell activation and presence of myoblasts.

### DETAILED DESCRIPTION OF SEVERAL ASPECTS

Disclosed herein are methods for treating a subject with a muscle wasting condition. Various muscle wasting conditions suitable for treatment by these methods are disclosed herein. The methods involve the use of MBV which can be prepared from extracellular matrix sources as disclosed herein. The method can include selecting the subject with the muscle wasting condition, and then treating the subject.

In some aspects, a method is disclosed for treating a subject with a muscle wasting condition that includes administering to the subject a composition having an effective amount of: (1) exogenous matrix bound nanovesicles (MBV) derived from extracellular matrix, wherein the MBV do not express CD63 and CD81 or are CD63^{lo}CD81^{lo} and wherein the MBV do not contain alkaline phosphatase: (2) myeloid progenitor cells or myeloid derived cells treated with the exogenous MBV; and/or (3) conditioned media or a fraction thereof obtained from a culture of macrophages cultured in the presence of the exogenous MBV, thereby treating the muscle wasting condition in the subject. The subject may be a human subject. The method can include selecting the subject with the muscle wasting condition, and then treating the subject.

In some aspects, a method is disclosed for promoting muscle regeneration or repair in a subject in need thereof. These methods include administering to the subject a composition comprising an effective amount of exogenous matrix bound nanovesicles (MBV) derived from extracellular matrix, wherein the MBV do not express CD63 and CD81 or are CD63^{lo}CD81^{lo} and wherein the MBV do not contain alkaline phosphatase, thereby to promote muscle regeneration in the subject.

The MBV may be derived from extracellular matrix of urinary bladder, small intestine, heart, dermis, liver, kidney, uterus, brain, blood vessel, lung, bone, muscle, pancreas, placenta, stomach, spleen, colon, adipose tissue, or esophagus. In some examples, the MBV are not derived from bone or cardiac ECM. For example, the MBV are derived from extracellular matrix of urinary bladder, small intestine, dermis, liver, kidney, uterus, brain, blood vessel, lung, muscle, pancreas, placenta, stomach, spleen, colon, adipose tissue, or esophagus. For example, the MBV may be derived from urinary bladder matrix (UBM), small intestinal submucosa (SIS), or urinary bladder submucosa (UBS). For example, the MBV may be derived from dermis. For example, the MBV are derived from extracellular matrix from a mammalian vertebrate selected from a human, monkey, pig, cow, or sheep.

For example, in one aspect, a subject having a muscle wasting disease is administered an effective amount of a composition comprising exogenous matrix bound nanovesicles (MBV) derived from extracellular matrix, wherein the MBV do not express CD63 and CD81 or are CD63^{lo}CD81^{lo} and wherein the MBV do not contain alkaline phosphatase.

In another aspect, a subject in need of muscle regeneration or repair is administered an effective amount of a composition comprising exogenous matrix bound nanovesicles (MBV) derived from extracellular matrix, wherein the MBV do not express CD63 and CD81 or are CD63^{lo}CD81^{lo} and wherein the MBV do not contain alkaline phosphatase.

The MBV may be administered to a subject by systemic administration, such as intravenous administration.

The exogenous MBV may be administered in a physiologically acceptable solution, such as, for example, saline at physiological pH. The exogenous MBV may be contained within an extracellular-matrix (ECM) hydrogel or pregel prepared from extracellular matrix (ECM) and the hydrogel or pregel is administered to the subject. The ECM hydrogel or pregel comprises solubilized ECM at a concentration of 1 mg/mL to 500 mg/mL, such as 1 mg/mL to 50 mg/mL. For example, the extracellular-matrix hydrogel or pregel may be an enzymatic ECM hydrogel or pregel and contains an inactivated protease, e.g., trypsin and/or pepsin. For example, the enzymatic hydrogel or pregel has a pH of about 7.0 to about 7.8. For example, the enzymatic pregel forms a gel at a temperature greater than about 25 °C In some examples, the ECM hydrogel is an acoustic hydrogel with a storage modulus (G′) of about 50 Pa to about 200 Pa, and a loss modulus (G″) of about 5 Pa to about 20 Pa, and a G′ to G″ ratio of about 4:1 to about 15:1 at 37 °C In some examples, the ECM in the hydrogel or pregel is not dialyzed.

The ECM hydrogel may be prepared from extracellular matrix of urinary bladder, small intestine, heart, dermis, liver, kidney, uterus, brain, blood vessel, lung, bone, muscle, pancreas, placenta, stomach, spleen, colon, adipose tissue, or esophagus. In some examples, the ECM hydrogel may be prepared from is derived from urinary bladder matrix (UBM), small intestinal submucosa (SIS), or urinary bladder submucosa (UBS). The ECM hydrogel may be prepared from dermis. The ECM hydrogel may be prepared from extracellular matrix from a mammalian vertebrate selected from a human, monkey, pig, cow, or sheep.

For example, in another aspect, a subject having a muscle wasting disease is administered an effective amount of a composition comprising myeloid progenitor cells or myeloid derived cells treated with the exogenous MBV. The myeloid progenitor cells or myeloid derived cells may be autologous to the subject. The myeloid progenitor cells or myeloid derived cells may be treated with the exogenous MBV in vitro, for example, in a cell culture. The myeloid progenitor cells or myeloid derived cells can then be isolated from the cell culture in order to be administered to the subject. The myeloid derived cells may be macrophages, monocytes, granulocytes, or myeloid progenitor cells. The myeloid progenitor cells or myeloid derived cells may be administered to the subject by systemic administration. For example, the myeloid progenitor cells or myeloid derived cells may be administered by intravenous administration. The myeloid progenitor cells or myeloid derived cells may be administered directly to the bone marrow cavity. The subject may be a human subject.

For example, in another aspect, a subject having a muscle wasting disease is administered an effective amount of a composition comprising conditioned media or a fraction thereof obtained from a culture of macrophages derived from the subject and cultured in the presence of the exogenous MBV. The macrophages may be autologous to the subject. The conditioned media may be purified by dialysis, size fractionation, and/or centrifugation prior to the conditioned media being administered to the subject. The subject may be a human subject.

In some aspects, the muscle wasting condition is a muscular dystrophy. For example, the muscular dystrophy is Becker Muscular Dystrophy, Congenital Muscular Dystrophy, Duchenne Muscular Dystrophy (DMD), Distal Muscular Dystrophy, Emery-Dreifuss Muscular Dystrophy, Facioscapulohumeral MD (FSHD), Limb-Girdle Muscular Dystrophy, Myotonic Muscular Dystrophy, Oculopharyngeal Muscular Dystrophy, Bethlem Myopathy, or Ullrich Congenital Muscular Dystrophy. For example, the muscle wasting condition is FSHD.

In some aspects, the muscle wasting condition is spinal muscular atrophy (SMA). For example, the SMA may be infantile progressive spinal muscular atrophy (SMA Type I), intermediate spinal muscular atrophy (SMA Type II), juvenile spinal muscular atrophy (SMA Type III), or adult spinal muscular atrophy (SMA Type IV).

In other aspects, the muscle wasting condition is sarcopenia or cachexia.

In some aspects, the methods disclosed herein for treating a subject with a muscle wasting condition increases myotube formation in the subject, maintain neuronal progenitor cells in an undifferentiated state in the subject and/or increase the migratory capacity of endothelial cells in the subject. The methods disclosed herein may also increase the growth of muscle tissue in the subject.

In other aspects, a composition for use in treating the methods disclosed herein is provided, where the composition provides an effective amount of (a) exogenous matrix bound nanovesicles (MBV) derived from extracellular matrix, wherein the MBV do not express CD63 and CD81 or are CD63^{lo}CD81^{lo} and wherein the MBV do not contain alkaline phosphatase; (b) myeloid progenitor cells or myeloid-derived cells treated with the exogenous MBV; and/or (c) conditioned media, or a fraction thereof, obtained from a macrophages cultured in the presence of the exogenous MBV.

In some aspects, the methods disclosed herein for promoting muscle regeneration or repair increase the number of satellite cells in the muscle of the subject where regeneration or repair is needed as compared to the number prior to administration of the MBV. In some aspects, the methods disclosed herein for promoting muscle regeneration or repair increase the number of myoblasts in the muscle of the subject where regeneration or repair is needed as compared to the number prior to administration of the MBV.

### TERMS

Unless otherwise noted, technical terms are used according to conventional usage. Definitions of many common terms in molecular biology may be found in Krebs er al. (eds.), Lewin's genes XII, published by Jones & Bartlett Learning, 2017. The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a MBV” includes single or plural MBVs and is considered equivalent to the phrase “comprising at least one MBV.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.” It is further to be understood that any and all molecular weight or molecular mass values, or proportions, given for compositions are approximate, and are provided for descriptive purposes, unless otherwise indicated. Dates of GENBANK® Accession Nos. referred to herein are the sequences available at least as early as Apr. 5, 2021. All references, patent applications and publications, and GENBANK® Accession numbers cited herein are incorporated by reference. Unless otherwise indicated, “about” indicates within five percent. Where the use of the term “about” is before a quantitative value, the present disclosure also includes the specific quantitative value itself. In case of conflict, the present specification, including explanations of terms, will control. In order to facilitate review of the various aspects of the disclosure, the following explanations of specific terms are provided:
Acid Protease: An enzyme that cleaves peptide bonds, wherein the enzyme has increased activity of cleaving peptide bonds in an acidic pH. For example and without limitation, acid proteases can include pepsin and trypsin.
Administration: The introduction of a composition (such as MBV or a pharmaceutical preparation that includes MBV) into a subject by a chosen route. The route can be local or systemic. For example, if the chosen route is intravenous, the composition is administered by introducing the composition into a vein of the subject. If the chosen route is local, the composition can be administered by introducing the composition directly into a tissue of the subject.
Animal: Living multi-cellular vertebrate organisms, a category that includes, for example, mammals and birds. The term “mammal” includes both human and non-human mammals. Similarly, the term “subject” includes both human and veterinary subjects.
Biocompatible: Any material, that, when implanted in a mammalian subject, does not provoke an adverse response in the subject. A biocompatible material, when introduced into an individual, is able to perform its intended function, and is not toxic or injurious to that individual, nor does it induce immunological rejection of the material in the subject.
Cachexia: A muscle wasting syndrome associated with an underlying illness, causing ongoing muscle loss that is not entirely reversed with nutritional supplementation, and may be accompanied by loss of fat mass. A range of diseases can cause cachexia, most commonly cancer, congestive heart failure, chronic obstructive pulmonary disease, chronic kidney disease, and AIDS. The primary features of cachexia include progressive depletion of muscle and fat mass, reduced food intake, abnormal metabolism of carbohydrate, protein, and fat, reduced quality of life, and increased physical impairment. Cachexia differs from weight loss due to malnutrition from malabsorption, anorexia nervosa, or anorexia due to major depressive disorder. Weight loss from inadequate caloric intake generally causes fat loss before muscle loss, whereas cachexia causes predominantly muscle wasting. Cachexia is also distinct from sarcopenia.
Centrifugation: The process whereby a centrifugal force is applied to a mixture, whereby more-dense components of the mixture migrate away from the axis of the centrifuge relative to other less-dense components in the mixture. The force that is applied to the mixture is a function of the speed of the centrifuge rotor, and the radius of the spin. In most applications, the force of the spin will result in a precipitate (a pellet) to gather at the bottom of the centrifuge tube, where the remaining solution is properly called a “supernate” or “supernatant.” In other similar applications, a density-based separation or “gradient centrifugation” technique is used to isolate a particular species from a mixture that contains components that are both more dense and less dense than the desired component.

During the circular motion of a centrifuge rotor, the force that is applied is the product of the radius and the angular velocity of the spin, where the force is traditionally expressed as an acceleration relative to “g,” the standard acceleration due to gravity at the Earth's surface. The centrifugal force that is applied is termed the “relative centrifugal force” (RCF), and is expressed in multiples of “g.”
Comminute (comminution and comminuting): The process of reducing larger particles into smaller particles, including, without limitation, by grinding, blending, shredding, slicing, milling, or cutting. ECM can be comminuted while in any form, including, but not limited to, hydrated forms, frozen, air-dried, lyophilized, powdered, or sheet-form. “Comminuted ECM” includes intact collagen. In some instances, comminuted ECM has not been subjected to ultrasound or enzymatic digestion, e.g., with a protease, such as an acid protease.
Conditioned medium or media: A medium containing a base medium and a cell secretome. The base medium is suitable for culturing cells. Conditioned medium is generally produced by culturing cells of interest (e.g., macrophages) in a base tissue culture medium such that the cells secrete active components (e.g. proteins and other factors) into the base medium, thereby producing conditioned media. The cells are typically removed from the conditioned medium, e.g., by centrifugation and/or other suitable methods prior to use of the conditioned media. The conditioned medium may be substantially cell free (e.g., less than 5% of the volume of the conditioned media) or completely cell free, such that it does not contain viable cells.
Contacting: Placement in direct physical association, which can be in solid or liquid form.
Cytokine: The term “cytokine” is used as a generic name for a diverse group of soluble proteins and peptides that act as humoral regulators at nano- to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to, tumor necrosis factor-α, interleukin (IL)-6, IL-10, IL-12, transforming growth factor, and interferon-γ.
Diagnosis: The process of identifying a disease by its signs, symptoms and results of various tests. The conclusion reached through that process is also called “a diagnosis.” Forms of diagnostic testing commonly performed include, without limitation, blood tests, medical imaging, and biopsy.
Enriched: A process whereby a component of interest, such as a nanovesicle, that is in a mixture has an increased ratio of the amount of that component to the amount of other components in that mixture after the enriching process as compared to before the enriching process.
Extracellular matrix (ECM): A complex mixture of structural and functional biomolecules and/or biomacromolecules including, but not limited to, structural proteins, specialized proteins, proteoglycans, glycosaminoglycans, and growth factors that surround and support cells within tissues and, unless otherwise indicated, is acellular. ECM preparations can be considered to be “decellularized” or “acellular,” meaning the cells have been removed from the source tissue through processes described herein and known in the art. By “ECM-derived material,” such as an “ECM-derived nanovesicle,” “Matrix bound nanovesicle,” “MBV” or “nanovesicle derived from an ECM” it is meant a nanovesicle that is prepared from a natural ECM or from an in vitro source wherein the ECM is produced by cultured cells. “Intact Extracellular Matrix” and “intact ECM” refers to an extracellular matrix that retains activity of its structural and non-structural biomolecules, including, but not limited to, collagens, elastins, laminins, glycosaminoglycans, proteoglycans, antimicrobials, chemoattractants, cytokines, and growth factors, such as, without limitation, comminuted ECM as described herein. The activity of the biomolecules within the ECM can be removed chemically or mechanically, for example, by cross-linking and/or by dialyzing the ECM. Intact ECM essentially has not been cross-linked and/or dialyzed, meaning that the ECM has not been subjected to a dialysis and/or a cross-linking process, or conditions other than processes that occur naturally during storage and handling of ECM prior to solubilization in making an enzymatic ECM hydrogel. Thus, ECM that is substantially cross-linked and/or dialyzed (in anything but a trivial manner which does not substantially affect the gelation and functional characteristics of the ECM in its uses described herein) is not considered to be “intact”.
Exogenous: Originating from a different source. Exogenous MBVs are produced separately, e.g., extracted from an ECM source, and added to an ECM hydrogel, that may or may not have endogenous MBV present in the ECM hydrogel. Exogenous MBV can be derived from the same tissue, or a different tissue, than the ECM used to make an ECM hydrogel. Exogenous MBV can be derived from the same species, or a different species, than the ECM used to make an ECM hydrogel.
Gel: A state of matter between liquid and solid, and is generally defined as a cross-linked polymer network swollen in a liquid medium. Typically, a gel is a two-phase colloidal dispersion containing both solid and liquid, wherein the amount of solid is greater than that in the two-phase colloidal dispersion referred to as a “sol.” As such, a “gel” has some of the properties of a liquid (i.e., the shape is resilient and deformable) and some of the properties of a solid (for example, the shape is discrete enough to maintain three dimensions on a two dimensional surface). “Gelation time,” also referred to as “gel time,” refers to the time it takes for a composition to become non-flowable under modest stress.
Gelation: The formation of a gel from a sol.
Hydrogel: A network of polymer chains that are hydrophilic, sometimes found as a colloidal gel in which water is the dispersion medium. Hydrogels are highly absorbent natural or synthetic polymeric networks. Hydrogels also possess a degree of flexibility similar to natural tissue. An “acoustic” hydrogel, such as an acoustic ECM hydrogel, is produced using ultrasound energy, for example, to solubilize ECM. The characteristics of these hydrogels are disclosed herein. For a hydrogel, the G′ (storage modulus) is typically about an order of magnitude greater than the G″ (loss modulus). An “enzymatic” ECM hydrogel, is produced by enzymatically digested ECM. The viscosity of an enzymatic hydrogel increases when warmed to physiological temperatures approaching about 37 °C For example, an enzymatic hydrogel is formed from an injectable solution at temperatures lower than 37 °C which forms a gel at a physiological temperature of 37 °C A “pregel” refers to an enzymatic hydrogel in the sol state, e.g., because the appropriate temperature for gelation has not yet been reached.
Improving muscular health: An improvement in muscular health compared with a preexisting state or compared with a state which would occur in the absence of treatment. For example, improving muscular health may include enhancing muscle regeneration, maintenance, or repair, or reducing or reversing degeneration of muscular tissue. Improving muscular health may also include prospectively treating a subject to prevent or reduce muscular damage, degeneration, or injury.
Isolated: An “isolated” biological component (such as a nucleic acid, protein cell, or nanovesicle) has been substantially separated or purified away from other biological components in the cell of the organism or the ECM, in which the component naturally occurs. Nucleic acids and proteins that have been “isolated” include nucleic acids and proteins purified by standard purification methods. MBV that have been isolated are removed from the fibrous materials of the ECM. The term also embraces nucleic acids and proteins prepared by recombinant expression in a host cell as well as chemically synthesized nucleic acids.
Isotonic Buffered Solution: A solution that is buffered to a pH between 7.0 and 7.8 and that has a balanced concentration of salts to promote an isotonic environment.
Lysyl oxidase (Lox): A copper-dependent enzyme that catalyzes formation of aldehydes from lysine residues in collagen and elastin precursors. These aldehydes are highly reactive, and undergo spontaneous chemical reactions with other lysyl oxidase-derived aldehyde residues, or with unmodified lysine residues. In vivo, this results in cross-linking of collagen and elastin, which plays a role in stabilization of collagen fibrils and for the integrity and elasticity of mature elastin. Complex cross-links are formed in collagen (pyridinolines derived from three lysine residues) and in elastin (desmosines derived from four lysine residues) that differ in structure. The genes encoding Lox enzymes have been cloned from a variety of organisms (Hamalainen et al., Genomics 11:508, 1991; Trackman et al., Biochemistry 29:4863, 1990; incorporated herein by reference). Residues 153-417 and residues 201-417 of the sequence of human lysyl oxidase have been shown to be important for catalytic function. There are four Lox-like isoforms, called LoxL1, LoxL2, LoxL3 and LoxL4.
Macrophage: A type of white blood cell that phagocytoses and degrades cellular debris, foreign substances, microbes, and cancer cells. In addition to their role in phagocytosis, these cells play an important role in development, tissue maintenance and repair, and in both innate and adaptive immunity in that they recruit and influence other cells including immune cells such as lymphocytes. Macrophages can exist in many phenotypes, including phenotypes that have been referred to as M1 and M2. Macrophages that perform primarily pro-inflammatory functions are called M1 macrophages (CD86⁺/CD68⁺), whereas macrophages that decrease inflammation and encourage and regulate tissue repair are called M2 macrophages (CD206⁺/CD68⁺). The markers that identify the various phenotypes of macrophages vary among species. It should be noted that macrophage phenotype is represented by a spectrum that ranges between the extremes of M1 and M2. F4/80 (encoded by the adhesion G protein coupled receptor E1 (ADGRE1) gene) is a macrophage marker, see GENBANK® Accession No. NP_001243181.1, Apr. 6, 2018, and NP_001965, Mar. 5, 2018, both incorporated herein by reference. Without wishing to be bound by theory, it is believed that MBV have the ability to modulate the phenotype of macrophages, leading to an increase in M2-like, regulatory, or pro-remodeling macrophages. The effect of MBV on macrophages is further characterized in PCT Publication No. WO 2017/151862A1, incorporated herein by reference in its entirety. In some aspects, MBV can be used to induce an M2 phenotype in macrophages and inhibit M1 macrophages in a subject.
MicroRNA: A small non-coding RNA that is about 17 to about 25 nucleotide bases in length, that post-transcriptionally regulates gene expression by typically repressing target mRNA translation. A microRNA (“miRNA” or “miR”) can function as negative regulators, such that greater amounts of a specific miRNA will correlates with lower levels of target gene expression. There are three forms of miRNAs, primary miRNAs (pri-miRNAs), premature miRNAs (pre-miRNAs), and mature miRNAs. Primary miRNAs (pri-miRNAs) are expressed as stem-loop structured transcripts of about a few hundred bases to over 1 kb. The pri-miRNA transcripts are cleaved in the nucleus by an RNase II endonuclease called Drosha that cleaves both strands of the stem near the base of the stem loop. Drosha cleaves the RNA duplex with staggered cuts, leaving a 5′ phosphate and 2 nucleotide overhang at the 3′ end. The cleavage product, the premature miRNA (pre-miRNA) is about 60 to about 110 nucleotides long with a hairpin structure formed in a fold-back manner. Pre-miRNA is transported from the nucleus to the cytoplasm by Ran-GTP and Exportin-5. Pre-miRNAs are processed further in the cytoplasm by another RNase II endonuclease called Dicer. Dicer recognizes the 5′ phosphate and 3′ overhang, and cleaves the loop off at the stem-loop junction to form miRNA duplexes. The miRNA duplex binds to the RNA-induced silencing complex (RISC), where the antisense strand is preferentially degraded and the sense strand mature miRNA directs RISC to its target site. It is the mature miRNA that is the biologically active form of the miRNA and is about 17 to about 25 nucleotides in length.
Muscular dystrophy: A term used to refer to a group of genetic disorders that lead to progressive muscle weakness. Muscular dystrophy can result in skeletal muscle weakness and defects in skeletal muscle proteins, leading to a variety of impaired physiological functions. No satisfactory treatment of muscular dystrophy exists. Existing treatments typically focus on ameliorating the effects of the disease and improving the patient's quality of life, such as through physical therapy, surgical interventions, or through the provision of orthopedic devices.

Mutated genes associated with muscular dystrophy are responsible for encoding a number of proteins associated with the costameric protein network. Such proteins include laminin-2, collagen, dystroglycan, integrins, caveolin-3, ankyrin, dystrophin, α-dystrobrevin, vinculin, plectin, BPAG1b, muscle LIM protein, desmin, actinin-associated LIM protein, α-actin, titin, telethonin, cypher, myotilin, and the sarcoglycan/sarcospan complex.

The most common form of muscular dystrophy is Duchenne muscular dystrophy (DMD), affecting 1 in 3,500 live male births. DMD is an X-linked recessive disorder characterized by a mutation in the gene that codes for dystrophin. Dystrophin is a cytoskeletal protein about 430 kDa in size. This protein works to connect the cell's cytoskeleton and extracellular matrix. The loss of dystrophin in DMD patients leads to a loss of muscle fiber attachment at the extracellular matrix during contraction, which ultimately leads to progressive fiber damage, membrane leakage and a loss of muscle function. Most patients die before they reach the age of 30 due to respiratory or cardiac failure.
Beckers muscular dystrophy (also known as Benign pseudohypertrophic muscular dystrophy) is related to Duchenne muscular dystrophy in that both result from a mutation in the dystrophin gene, but in Duchenne muscular dystrophy no functional dystrophin is produced making DMD much more severe than BMD. BMD is an X-linked recessive inherited disorder characterized by slowly progressive muscle weakness of the legs and pelvis. BMD is a type of dystrophinopathy, which includes a spectrum of muscle diseases in which there is insufficient dystrophin produced in the muscle cells, results in instability in the structure of muscle cell membrane. This is caused by mutations in the dystrophin gene. The pattern of symptom development of BMD is similar to DMD, but with a later, and much slower rate of progression.
“Congenital muscular dystrophy” is caused by gene mutations affecting the production of other costameric proteins. Merosin deficient congenital muscular dystrophy (MDC1A) is a congenital muscular dystrophy due to a genetic mutation in the LAMA2 gene which results in lack of or complete loss of laminin-α2 protein. This loss of laminin-α2 leads to an absence of laminins-211/221. Laninins-211/221 are major components of the extracellular matrix and play a key role in muscle cell development. During muscle cell differentiation laminin binds to the α7β1 integrin. Without laminin-α2, muscle fibers are unable to adhere to the basement membrane and myotubes undergo apoptosis. Muscle regeneration also fails, leading to a loss of muscle repair and an increase in muscle fibrosis and inflammation. This chronic tissue injury is a major cause of morbidity and mortality in MDC1A.

Congenital Muscular Dystrophies (CMD) and Limb-Girdle muscular dystrophy (LGMD) are common forms of highly heterogeneous muscular dystrophies which can be distinguished by their age at onset. In CMD, onset of symptoms is at birth or within the first 6 months of life; in LGMD onset of symptoms is in late childhood, adolescence or even adult life. Inheritance in LGMD can be autosomal dominant (LGMD type 1) or autosomal recessive (LGMD type 2), CMD is recessively inherited. CMD and LGMD can overlap both clinically and genetically
MDC1A is a progressive muscle wasting disease that results in children being confined to a wheelchair, requiring ventilator assistance to breathe and premature death. Symptoms are detected at birth with poor muscle tone and “floppy” baby syndrome. DMD, BMD and LGMD are progressive muscle degenerative diseases usually diagnosed at 3-5 years of age when children show developmental delay including ability to walk and climb stairs. The disease is progressive and children are usually confined to a wheelchair in their teens and require ventilator assistance.
Facioscapulohumeral muscular dystrophy (FSHD) is a form of muscular dystrophy associated with progressive muscle weakness and loss of muscle tissue. Unlike DMD and BMD which mainly affect the lower body, FSHD affects the upper body mainly the face, shoulder and upper arm muscles. However, it can affect muscles around the pelvis, hips, and lower leg. Symptoms for FSHD often do not appear until age 10-26, but it is not uncommon for symptoms to appear much later. In some cases, symptoms never develop. Symptoms are usually mild and very slowly become worse. Facial muscle weakness is common, and may include eyelid drooping, inability to whistle, decreased facial expression, depressed or angry facial expression, difficulty pronouncing words, shoulder muscle weakness (leading to deformities such as pronounced shoulder blades (scapular winging) and sloping shoulders), weakness of the lower, hearing loss and possible heart conditions. FSHD is caused by a mutation that leads to aberrant expression of the double homeobox protein 4 gene (DUX4), leading to protein expression that is toxic to muscle cells.
Myeloid cell: “Myeloid cell” or “myeloid derived cell” refers to cells derived from a myeloid progenitor cell and includes, e.g., granulocytes (e.g., basophils, neutrophils, eosinophils) and monocytes. As monocytes may differentiate into macrophages or dendritic cells, macrophages and dendritic cells are also considered myeloid cells or myeloid derived cells.
Myoblast: Proliferating satellites cells and their progeny are referred to as myoblasts, also known as myogenic progenitor cells. Myoblasts fuse together to produce new myofibers and can fuse to existing myofibers of skeletal muscle tissue, leading to skeletal muscle regeneration.
Muscle cell: A myocyte, which is a mature contractile cell in the muscle of an animal. There are three types of muscle: skeletal, smooth, and cardiac. A skeletal muscle cell is long and threadlike with many nuclei and is called a muscle fiber, and develops from myoblasts. Skeletal muscle cells form by fusion of myoblasts to produce multinucleated cells (syncytia) in a process known as myogenesis. Skeletal muscle cells contain myofibrils, formed of filaments, and sarcomeres and form striated muscle tissue. The filaments of myofibrils, myofilaments, consist of three types, thick, thin, and elastic filaments. Thin filaments consist primarily of the protein actin, coiled with nebulin filaments, hick filaments consist primarily of the protein myosin, that is responsible for force generation. Elastic filaments are made up of a protein called titin and hold the thick filaments in place. The actin and myosin filaments each have a specific and constant length on the order of a few micrometers, far less than the length of the elongated muscle cell (a few millimeters in the case of human skeletal muscle cells). The filaments are organized into repeated subunits along the length of the myofibril. The muscle cell is nearly filled with myofibrils running parallel to each other on the long axis of the cell.
Nanovesicle: An extracellular vesicle that is a nanoparticle of about 10 to about 1,000 nm in diameter. Nanovesicles are lipid membrane bound particles that carry biologically active signaling molecules (e.g. microRNAs, proteins) among other molecules. Generally, the nanovesicle is limited by a lipid bilayer, and the biological molecules are enclosed and/or can be embedded in the bilayer. Thus, a nanovesicle includes a lumen surrounded by plasma membrane. The different types of vesicles can be distinguished based on diameter, subcellular origin, density, shape, sedimentation rate, lipid composition, protein markers, nucleic acid content and origin, such as from the extracellular matrix or secreted. A nanovesicle can be identified by its origin, such as a matrix bound nanovesicle from an ECM (see above), protein content and/or the miR content.

An “exosome” or “liquid phase extracellular vesicle (EV)” is a membranous vesicle which is secreted by a cell, and ranges in diameter from 10 to 150 nm. Generally, late endosomes or multivesicular bodies contain intralumenal vesicles which are formed by the inward budding and scission of vesicles from the limited endosomal membrane into these enclosed vesicles. These intralumenal vesicles are then released from the multivesicular body lumen into the extracellular environment, typically into a body fluid such as blood, cerebrospinal fluid or saliva, during exocytosis upon fusion with the plasma membrane. An exosome is created intracellularly when a segment of membrane invaginates and is endocytosed. The internalized segments which are broken into smaller vesicles and ultimately expelled from the cell contain proteins and RNA molecules such as mRNA and miRNA. Plasma-derived exosomes largely lack ribosomal RNA. Extra-cellular matrix derived exosomes include specific miRNA and protein components, and have been shown to be present in virtually every body fluid such as blood, urine, saliva, semen, and cerebrospinal fluid. Exosomes can express CD11c, CD63, CD81, and/or CD9, and thus can be CD11c⁺ and/or CD63⁺ and/or C81⁺ and/or CD9⁺. Exosomes do not have high levels of lysyl oxidase on their surface.

A “nanovesicle derived from an ECM,” “matrix bound nanovesicle,” “MBV” or an “ECM-derived nanovesicle” all refer to the same membrane bound particles, ranging in size from 10 nm-1000 nm, present in the extracellular matrix, which contain biologically active signaling molecules such as protein, lipids, nucleic acid, growth factors and cytokines that influence cell behavior. The terms are interchangeable, and refer to the same vesicles. These nanovesicles are embedded within, and bound to, the ECM and are not simply attached to the surface or circulating freely in body fluids. These nanovesicles are resistant to harsh isolation conditions, such as freeze-thawing and digestion with proteases such as pepsin, elastase, hyaluronidase, proteinase K, and collagenase, and digestion with detergents. MBV are distinct from other extracellular vesicles including exosomes and have a phospholipid composition distinct from exosomes. MBV are distinct from bone matrix vesicles which express alkaline phosphatase, as MBV do not express alkaline phosphatase. In certain circumstances, MBV can also be distinguished from exosomes based on the absence of certain markers commonly attributed to exosomes.

In some aspects, MBV are characterized by one or more of the following features of protein expression or lipid content:
(i) MBV may not express one or more of CD63 and/or CD81 and/or CD9 or have low or barely detectable levels of CD63 and/or CD81 and/or CD9 (CD63^{lo} and/or CD81^{lo} and/or CD9^{lo})(see, e.g., Example 1) compared with other vesicles, such as exosomes. A variety of methods can be used to distinguish low, barely detectable, or absent expression of CD63 and/or CD81 and/or CD9 in MBV, for example, antibody-based methods, such as western blotting or flow cytometry (see, e.g., Bashashati and Brinkman, Adv Bioinformatics, 2009: 584603). In some aspects, MBV expression of CD63 and/or CD81 and/or CD9 is considered low or barely detectable compared with other vesicles where the expression of CD63 and/or CD81 and/or CD9 in MBV is at least one standard deviation or at least two standard deviations below the mean expression of other vesicles, such as exosomes;
(ii) MBV have a phospholipid content wherein at least 55% of total phospholipids comprise phosphatidylcholine (PC) and phosphatidyl inositol (PI) in combination;
(iii) MBV have a phospholipid content wherein 10% or less of total phospholipids comprise sphingomyelin (SM);
(iv) MBV have a phospholipid content wherein 20% or less of total phospholipids comprise phosphatidylethanolamine (PE);
(v) MBV have a phospholipid content wherein 15% or greater of the total phospholipid content comprises phosphatidylinositol (PI) with the percent representing the percent of lipid concentration.

In some aspects, MBV are characterized by all of the following features:
(i) do not express one or more of CD63 and/or CD81 and/or CD9 or have low or barely detectable levels of CD63 and/or CD81 and/or CD9 (CD63^{lo} and/or CD81^{lo} and/or CD9^{lo})(as further described above);
(ii) a phospholipid content wherein at least 55% of total phospholipids comprise phosphatidylcholine (PC) and phosphatidyl inositol (PI) in combination;
(iii) a phospholipid content wherein 10% or less of total phospholipids comprise sphingomyelin (SM);
(iv) a phospholipid content wherein 20% or less of total phospholipids comprise phosphatidylethanolamine (PE); and
(v) a phospholipid content wherein 15% or greater of the total phospholipid content is phosphatidylinositol (PI).

In some aspects, MBV are characterized by all of the following features:
(i) a phospholipid content wherein at least 55% of total phospholipids comprise phosphatidylcholine (PC) and phosphatidyl inositol (PI) in combination;
(ii) a phospholipid content wherein 10% or less of total phospholipids comprise sphingomyelin (SM);
(iii) a phospholipid content wherein 20% or less of total phospholipids comprise phosphatidylethanolamine (PE); and
(iv) a phospholipid content wherein 15% or greater of the total phospholipid content is phosphatidylinositol (PI).

In some aspects, MBV are characterized by one or more of the following features:
(i) a phospholipid content wherein at least 55% of total phospholipids comprise phosphatidylcholine (PC) and phosphatidyl inositol (PI) in combination;
(ii) a phospholipid content wherein 10% or less of total phospholipids comprise sphingomyelin (SM);
(iii) a phospholipid content wherein 20% or less of total phospholipids comprise phosphatidylethanolamine (PE); and
(iv) a phospholipid content wherein 15% or greater of the total phospholipid content is phosphatidylinositol (PI).

In some aspects, MBV are characterized by one or more of the following features:
(i) do not contain detectable levels of alkaline phosphatase;
(ii) do not contain detectable levels of osteopontin;
(iii) do not contain detectable levels of osteoprotegerin;
(iv) do not contain detectable levels of complement C5; and/or
(v) do not contain detectable levels of c-reactive protein.

In some aspects, MBV contain IL33 and are IL33⁺.

The ECM from which MBV are isolated can be an ECM from a tissue, can be produced from cells in culture, or can be purchased from a commercial source.
Pharmaceutically acceptable carriers: The pharmaceutically acceptable carriers useful in the claimed pharmaceutical preparations are conventional. Remington's Pharmaceutical Sciences, by E. W. Martin, Mack Publishing Co., Easton, PA, 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically-neutral carriers, pharmaceutical preparations to be administered can contain minor amounts of non-toxic auxiliary substances, such as wetting or emulsifying agents, preservatives, and pH buffering agents and the like, for example sodium acetate or sorbitan monolaurate.
Pharmaceutical agent: A chemical compound or composition capable of inducing a desired therapeutic or prophylactic effect when properly administered to a subject or a cell.
Phospholipid: A class of lipids having a structure consisting of two hydrophobic fatty acid tails and a hydrophilic head consisting of a phosphate group. Major classes of phospholipids include phosphatidylcholine (PC), phosphatidylethanolamine (PE), phosphatidylinositol (PI), phosphatidylserine (PS), phosphatidylglycerol (PG), sphingomyelin (SM), cardiolipin (CL), phosphatidic acid (PA), and bis-monoacylglycerophosphate (BMP). Phospholipids can be measured in a variety of ways. For example, liquid chromatography-mass spectrometry (LC-MS) based global lipidomics and redox lipidomics can be used. In some aspects, specific phospholipid content is indicated as the percent concentration of the total phospholipids (such as total phospholipids in MBV), where the percent concentration is weight/weight (w/w).
Polynucleotide: A nucleic acid sequence (such as a linear sequence) of any length. Therefore, a polynucleotide includes oligonucleotides, and also gene sequences found in chromosomes. An “oligonucleotide” is a plurality of joined nucleotides joined by native phosphodiester bonds. An oligonucleotide is a polynucleotide of between 6 and 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.
Prophylactic: as used herein refers to a medication or a treatment designed and used to prevent a disease or disorder from occurring. As used herein, the terms “prophylactic” and “prevention” are used interchangeably.
Purified: The term “purified” does not require absolute purity; rather, it is intended as a relative term. Thus, for example, a purified nucleic acid molecule preparation is one in which the nucleic referred to is more pure than the nucleic in its natural environment within a cell. For example, a preparation of a nucleic acid is purified such that the nucleic acid represents at least 50% of the total protein content of the preparation. Similarly, a purified MBV preparation is one in which the exosome is more pure than in an environment including cells, wherein there are microvesicles and exosomes. A purified population of nucleic acids or MBV is greater than about 90%, 91%, 92%, 93%, 94%, 95%, 96%, 97%, 98%, 99% or 100% pure, or free other nucleic acids or cellular components, respectively.
Preventing or treating a disease: “Preventing” a disease refers to inhibiting the development of a disease, for example in a person who is known to have a predisposition to a disease. An example of a person with a known predisposition is someone with a history of a disease in the family, or who has been exposed to factors that predispose the subject to a condition. “Treatment” refers to a therapeutic intervention that ameliorates a sign or symptom of a disease or pathological condition after it has begun to develop.
Regeneration: When referring to skeletal muscle, refers to the formation of new skeletal muscle tissue. Regeneration is a natural processing occurring in response to injury or stress or other event that causes loss of existing skeletal muscle in a subject. Skeletal muscle regeneration occurs through a process initiated by muscle stem cells (satellite cells) that leads to production of new myofibers and repair of existing myofibers. The presence of satellite cells and myoblasts in muscle tissue is indicative that muscle tissue is in a state of regeneration.
Sarcopenia: The age-related progressive loss of muscle mass and strength. The main symptom of the condition is muscle weakness. Sarcopenia affects both males and females equally; the incidence increases with age. In humans, sarcopenia generally affects individuals aged 60 and older. The disease affects both sexes equally. This condition is characterized by the degenerative loss of skeletal muscle mass, quality, and strength. The rate of muscle loss is dependent on exercise level, co-morbidities, nutrition and other factors. The pathologic changes of sarcopenia include a reduction in muscle tissue quality as reflected in the replacement of muscle fibers with fat, an increase in fibrosis, changes in muscle metabolism, oxidative stress, and degeneration of the neuromuscular junctions. Sarcopenia is diagnosed when a patient has muscle mass that is at least two standard deviations below the relevant population mean and has a slow walking speed. A subject with this condition has the presence of low muscle mass and either low muscular strength or low physical performance.
Satellite cells: Satellite cells are myogenic (muscle) stem cells that play a critical role in muscle fiber maintenance, remodeling, and repair. These precursors to skeletal muscle cells are mononucleated and found between the basement membrane and plasma membrane of the muscle fiber. Normally quiescent in adult muscle, they can proliferate in response to injury or stress, giving rise to regenerated muscle and more satellite cells.
Solubilized ECM: ECM that has been treated with ultrasonic cavitation or enzymatic digestion thereby causing micro-structural changes, such as by physical disruption of protein aggregates or digestion, respectively.
Spinal Muscular Atrophy: An inherited and acquired central nervous system (CNS) neurodegenerative disease characterized by progressive motor neuron loss in the spinal cord and brainstem causing muscle weakness and muscle atrophy. The most common form of SMA is caused by mutations in the Survival Motor Neuron (SMN) gene and manifests over a wide range of severity affecting infants through adults (Crawford and Pardo, Neurobiol. Dis., 1996, 3:97).

Specifically, SMA is triggered by a genetic mutation in the survival motoneuron 1 (SMN1) gene where a mutation of the SMN1 gene prevents the normal expression of the SMN protein. In most cases, SMA is diagnosed based on clinical symptoms and by the presence of at least one copy of the SMN1 gene test. However, in approximately 5% of cases SMA is caused by mutation in genes other than the inactivation of SMN 1. In some cases, when the SMN 1 gene test is not feasible or does not show any abnormality, other tests such as an electromyography (EMG) or muscle biopsy may be indicated.

Strikingly, even though the SMN1 gene is ubiquitously expressed in all MNs, not all muscles are affected. SMA specially affects lower limbs and, in the more severe cases, respiratory function. Affected motoneurons in SMA patients are less capable of producing sustained firing and can degrade over time leading to motoneuron (MN) death. Experiments in mouse models have shown that the insufficient expression of SMN protein first produces the dysfunction and, in the late stage of the disease, the death of MNs. MN dysfunction in SMA patients is produced by a non-cell-autonomous mechanism and it is independent of MN death. Thus, MN dysfunction and their death in SMA patients are two independent processes.

SMA severity ranges from respiratory failure in the neonatal period (type 1-2) to mild muscle weakness noticed in adulthood (type 4). Infantile SMA is the most severe form of this neurodegenerative disorder. Symptoms include muscle weakness, poor muscle tone, weak cry, limpness or a tendency to flop, difficulty sucking or swallowing, accumulation of secretions in the lungs or throat, feeding difficulties, and increased susceptibility to respiratory tract infections. The legs tend to be weaker than the arms and developmental milestones, such as lifting the head or sitting up, cannot be reached. In general, the earlier the symptoms appear, the shorter the lifespan. As the motor neuron cells deteriorate, symptoms appear shortly afterward. The severe forms of the disease are fatal. The course of SMA is directly related to the rate of motor neuron cell deterioration and the resulting severity of weakness. Infants with a severe form of SMA frequently succumb to respiratory disease due to weakness in the muscles that support breathing. Children with milder forms of SMA live longer, although they may need extensive medical support, especially those at the more severe end of the spectrum. The clinical spectrum of SMA disorders has been divided into the following five groups.
1) Type 0 SMA (In Utero SMA) is the most severe form of the disease and begins before birth. Usually, the first symptom of Type 0 SMA is reduced movement of the fetus that can first be observed between 30 and 36 weeks of pregnancy. After birth, these newborns have little movement and have difficulties with swallowing and breathing.
2) Type 1 SMA (Infantile SMA or Werdnig-Hoffmann disease) presents symptoms between 0 and 6 months. This form of SMA is also very severe. Patients never achieve the ability to sit, and death usually occurs within the first 2 years without ventilatory support.
3) Type 2 SMA (Intermediate SMA) has an age of onset at 7-18 months. Patients achieve the ability to sit unsupported, but never stand or walk unaided. Prognosis in this group is largely dependent on the degree of respiratory involvement.
4) Type 3 SMA (Juvenile SMA or Kugelberg-Welander disease) is generally diagnosed after 18 months. Type 3 SMA individuals are able to walk independently at some point during their disease course but often become wheelchair-bound during youth or adulthood.
5) Type 4 SMA (Adult onset SMA). Weakness usually begins in late adolescence in the tongue, hands, or feet, then progresses to other areas of the body. The course of adult SMA is much slower and has little or no impact on life expectancy.
Subject: Human and non-human animals, including all vertebrates, such as mammals and non-mammals, such as non-human primates, mice, rabbits, sheep, dogs, cats, horses, cows, chickens, amphibians, and reptiles. In some aspects of the described methods, the subject is a human. “Subject” is used interchangeably with the term “patient.” A subject may be an individual diagnosed with a high risk of developing a disease or disorder, for example, an infectious disease or disorder (e.g., an immunocompromised individual, a healthcare professional), someone who has been diagnosed with a disease or disorder, for example, an infectious disease or disorder, someone who previously suffered from a disease or disorder, for example, an infectious disease or disorder, or an individual evaluated for symptoms or indications of a disease or disorder, for example, an infectious disease or disorder.
Therapeutically effective amount: A quantity of a specific substance, such as an MBV, sufficient to achieve a desired effect in a subject being treated. When administered to a subject, a dosage will generally be used that will achieve target tissue concentrations (for example, in the lung) that has been shown to achieve a desired in vitro effect.
Thermoreversible hydrogel: Hydrogel formed due to entanglement of polymer chains wherein the viscosity changes at a characteristic temperature of gelation. The disclosed acoustic ECM hydrogels are thermoreversible hydrogels that show gelation (sol to gel transition) upon cooling.
Topical application: A topically applied agent is applied only in a specific area, and not throughout the body. In particular examples the composition is applied to the skin or the eye in an area where hemostasis is desired. For example the pharmaceutical composition can be applied in a topical preparation to a wound, such as an epithelial wound or defect, for example a traumatic or surgical wound, such as a skin or corneal abrasion or surgical incision.
Total phospholipid content: “Total phospholipids” or “total phospholipid content”, as used herein, with respect to MBV, refers to the sum of all phospholipids present in a given quantity of isolated MBV, i.e., MBV isolated from the ECM. MBV can be isolated, for example, by enzymatic digestion of decellularized ECM and differential centrifugation. The total phospholipid content can be determined by methods such as LC-MS based global lipidomics and redox lipidomics. The total phospholipid content is measured by weight. A percentage of the total phospholipid content refers to a percent concentration on a weight/weight basis
Transplanting: The placement of a biocompatible substrate, such as an MBV, into a subject in need thereof.
Treating, Treatment, and Therapy: Any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, or improving a subject's physical or mental well-being. The treatment may be assessed by objective or subjective parameters; including the results of a physical examination, neurological examination, or psychiatric evaluations.
Ultrasonication: The process of exposing ultrasonic waves with a frequency higher than 20 kHz.

### Matrix Bound Nanovesicles (MBV) Derived from an Extracellular Matrix (ECM)

Nanovesicles derived from ECM (also called matrix bound nanovesicles, “MBV”) are generally described in PCT Publication Nos. WO 2017/151862, WO 2018/204848, and WO 2019/213482, all incorporated herein by reference. It is disclosed that MBV are embedded in the extracellular matrix. These MBV can be isolated and are biologically active. MBV do not express CD63 and CD81 or are CD63^{lo}CD81^{lo} and do not contain alkaline phosphatase. The MBV can contain IL-33. These MBV can be used for therapeutic purposes. In some aspects, the MBV do not contain alkaline phosphatase, osteopontin, osteoprogeterin, complement C5, and/or c-reactive protein.

An extracellular matrix is a complex mixture of structural and functional biomolecules and/or biomacromolecules including, but not limited to, structural proteins, specialized proteins, proteoglycans, glycosaminoglycans, and growth factors that surround and support cells within mammalian tissues and, unless otherwise indicated, is acellular. Generally, the disclosed MBV are embedded in any type of extracellular matrix (ECM), and can be isolated from this location. Thus, MBV are not detachably present on the surface of the ECM, and are not exosomes (also known as extracellular vesicles or EV).

Extracellular matrices are disclosed, for example and without limitation, in U.S. Pat. Nos. 4,902,508; 4,956,178; 5,281,422; 5,352,463; 5,372,821; 5,554,389; 5,573,784; 5,645,860; 5,771,969; 5,753,267; 5,762,966; 5,866,414; 6,099,567; 6,485,723; 6,576,265; 6,579,538; 6,696,270; 6,783,776; 6,793,939; 6,849,273; 6,852,339; 6,861,074; 6,887,495; 6,890,562; 6,890,563; 6,890,564; and 6,893,666; each of which is incorporated by reference in its entirety). However, an ECM can be produced from any tissue, or from any in vitro source wherein the ECM is produced by cultured cells and comprises one or more polymeric components (constituents) of native ECM. ECM preparations can be considered to be “decellularized” or “acellular”, meaning the cells have been removed from the source tissue or culture.

In some aspects, the ECM is isolated from a vertebrate animal, for example, from a mammalian vertebrate animal including, but not limited to, human, monkey, pig, cow, sheep, etc. The ECM may be derived from any organ or tissue, including without limitation, urinary bladder, intestine (such as small intestine or large intestine), heart, dermis, liver, kidney, uterus, brain, blood vessel, lung, bone, muscle, pancreas, placenta, stomach, spleen, colon, adipose tissue, or esophagus. In specific non-limiting examples, the extracellular matrix is isolated from esophageal tissue, urinary bladder (such as urinary bladder matrix (UBM) or urinary bladder submucosa (UBS)), small intestinal submucosa (SIS), dermis, umbilical cord, pericardium, cardiac tissue, or skeletal muscle. For example, the ECM is UBM, or the ECM is SIS, or the ECM is UBS, or the ECM is dermis. The ECM can comprise any portion or tissue obtained from an organ, including, for example and without limitation, submucosa, epithelial basement membrane, tunica propria, etc. In one non-limiting aspect, the ECM is isolated from urinary bladder. In some aspects, the ECM is from a human subject. In other aspects, the ECM is from a porcine subject. In some aspect, the ECM is not porcine ECM. In some aspects, the ECM is not porcine UBM.

The ECM may or may not include the basement membrane. In another non-limiting aspect, the ECM includes at least a portion of the basement membrane. The ECM material may or may not retain some of the cellular elements that comprised the original tissue such as capillary endothelial cells or fibrocytes. In some aspects, the ECM contains both a basement membrane surface and a non-basement membrane surface.

In some aspects, the ECM is harvested from porcine urinary bladders (also known as urinary bladder matrix or UBM). Briefly, the ECM is prepared by removing the urinary bladder tissue from a mammal, such as a pig, and trimming residual external connective tissues, including adipose tissue. All residual urine is removed by repeated washes with tap water. The tissue is delaminated by first soaking the tissue in a de-epithelializing solution, for example and without limitation, hypertonic saline (e.g., 1.0 N saline), for periods of time ranging from ten minutes to four hours. Exposure to hypertonic saline solution removes the epithelial cells from the underlying basement membrane. Optionally, a calcium chelating agent may be added to the saline solution. The tissue remaining after the initial delamination procedure includes the epithelial basement membrane and tissue layers abluminal to the epithelial basement membrane. The relatively fragile epithelial basement membrane is invariably damaged and removed by any mechanical abrasion on the luminal surface. This tissue is next subjected to further treatment to remove most of the abluminal tissues but maintain the epithelial basement membrane and the tunica propria. The outer serosal, adventitial, tunica muscularis mucosa, tunica submucosa and most of the muscularis mucosa are removed from the remaining deepithelialized tissue by mechanical abrasion or by a combination of enzymatic treatment (e.g., using trypsin or collagenase) followed by hydration, and abrasion. Mechanical removal of these tissues is accomplished by removal of mesenteric tissues with, for example and without limitation, Adson-Brown forceps and Metzenbaum scissors and wiping away the tunica muscularis and tunica submucosa using a longitudinal wiping motion with a scalpel handle or other rigid object wrapped in moistened gauze. Automated robotic procedures involving cutting blades, lasers and other methods of tissue separation are also contemplated. After these tissues are removed, the resulting ECM consists mainly of epithelial basement membrane and subjacent tunica propria.

In another aspect, the ECM is prepared by abrading porcine bladder tissue to remove the outer layers including both the tunica serosa and the tunica muscularis using a longitudinal wiping motion with a scalpel handle and moistened gauze. Following eversion of the tissue segment, the luminal portion of the tunica mucosa is delaminated from the underlying tissue using the same wiping motion. Care is taken to prevent perforation of the submucosa. After these tissues are removed, the resulting ECM consists mainly of the tunica submucosa (see FIG. 2 of U.S. Pat. No. 9,277,999, which is incorporated herein by reference).

ECM can also be prepared as a powder. Such powder can be made according to the method of Gilbert et al., Biomaterials 26 (2005) 1431-1435, herein incorporated by reference in its entirety. For example, UBM sheets can be lyophilized and then chopped into small sheets for immersion in liquid nitrogen. The snap frozen material can then be comminuted so that particles are small enough to be placed in a rotary knife mill, where the ECM is powdered. Similarly, by precipitating NaCl within the ECM tissue the material will fracture into uniformly sized particles, which can be snap frozen, lyophilized, and powdered.

In one non-limiting aspect, the ECM is derived from small intestinal submucosa or SIS. Commercially available preparations include, but are not limited to, SURGISIS™, SURGISIS-ES™, STRATASIS™, and STRATASIS-ES™ (Cook Urological Inc.; Indianapolis, Ind.) and GRAFTPATCH™ (Organogenesis Inc.; Canton Mass.). In another non-limiting aspect, the ECM is derived from dermis. Commercially available preparations include, but are not limited to PELVICOL™ (sold as PERMACOL™ in Europe; Bard, Covington, Ga.), REPLIFORM™ (Microvasive; Boston, Mass.) and ALLODERM™ (LifeCell; Branchburg, N.J.). In another aspect, the ECM is derived from urinary bladder. Commercially available preparations include, but are not limited to UBM (ACell Corporation; Jessup, Md.).

MBV can be derived from (released from) an extracellular matrix using the methods disclosed below. For example, MBV may be obtained from extracellular matrix according to the methods disclosed in U.S. Patent Application Publication No. 2019/0117837, the contents of which are incorporated by reference herein for all purposes. In some aspects, the ECM is digested with an enzyme, such as pepsin, collagenase, elastase, hyaluronidase, and/or proteinase K, and the MBV are isolated. In other aspects, the MBV are released and separated from the ECM by changing the pH with solutions such as glycine HCL, citric acid, ammonium hydroxide, use of chelating agents such as, but not limited to, EDTA, EGTA, by ionic strength and or chaotropic effects with the use of salts such as, but not limited to potassium chloride (KCl), sodium chloride, magnesium chloride, sodium iodide, sodium thiocyanate, or by exposing ECM to denaturing conditions like guanidine HCl or Urea.

The MBV may be derived from extracellular matrix of urinary bladder, small intestine, heart, dermis, liver, kidney, uterus, brain, blood vessel, lung, bone, muscle, pancreas, placenta, stomach, spleen, colon, adipose tissue, and/or esophagus. In specific non-limiting examples, the MBV are derived from urinary bladder matrix (UBM), small intestinal submucosa (STS), or urinary bladder submucosa (UBS). In one aspect, the MBV are derived from dermis. In another aspect, the MBV are derived from UBM. In further aspects, the MBV are derived from extracellular matrix from a mammalian vertebrate selected from a human, monkey, pig, cow, or sheep. In specific non-limiting examples, the MBV are from a non-human mammal. In some aspects, the MBV are not derived from bone ECM. In some aspects, the MBV are not derived from heart (cardiac) ECM. In some aspects, the MBV are not derived from heart (cardiac) ECM or bone ECM. In particular aspects, the MBV are prepared following digestion of an ECM with an enzyme, such as pepsin, elastase, hyaluronidase, proteinase K, salt solutions, and/or collagenase, or combinations thereof. The ECM can be freeze-thawed, or subject to mechanical degradation.

In some aspects, expression of CD63, CD81, and/or CD9 cannot be detected on the MBV. Thus, in some aspects the MBV do not express CD63 and/or CD81 and/or CD9. In one specific example, CD63, CD81, and CD9 cannot be detected on the nanovesicles. In other aspects, the MBV have barely detectable levels of CD63, CD81, and CD9, such as that detectable by Western blot. These MBV are CD63^{lo}CD81^{lo}CD9^{lo}. In other aspects, MBV do not express detectable levels of one or more of CD63, CD81, or CD9. In other aspects, MBV express barely detectable levels of one or more of CD63, CD81, or CD9. One of skill in the art can readily identify MBV that are CD63^{lo} and/or CD81^{lo} and/or CD9^{lo}, using, for example, antibodies that specifically bind CD63, CD81, and CD9. A low level of these markers can be established using procedures such as fluorescent activated cell sorting (FACS) and fluorescently labeled antibodies to determine a threshold for low and high amounts of CD63, CD81, and CD9. In some examples, the disclosed MBV do not contain detectable alkaline phosphatase, osteopontin, osteoprogeterin, complement C5, and/or c-reactive protein. The disclosed MBV differ from nanovesicles, such as exosomes that may be transiently attached to the surface of the ECM due to their presence in biological fluids, as MBV in vivo are bound to the ECM and not found in biological fluids.

MBV have distinctive phospholipid content, for example, in comparison to exosomes. In some aspects, the total phospholipid content of the MBV is at least 50%, 55%, 60%, 65%, 70%, 75%, 85%, or 90%, or about 50%-90%, 50%-65%, 50%-60%, 50%-70%, 60%-70%, 60%-90%, or 70%-90% of phosphatidylcholine (PC) and phosphatidyl inositol (PI) in combination. In specific aspects, the total phospholipid content of the MBV is at least 55% of phosphatidylcholine (PC) and phosphatidyl inositol (PI) in combination. In specific aspects, the total phospholipid content of the MBV is at least 60% of phosphatidylcholine (PC) and phosphatidyl inositol (PI) in combination. In some aspects, the phospholipid content of the MBV comprises a phosphatidylcholine (PC) to phosphatidyl inositol (PI) ratio of less than 8:1 (for example, less than 7:1, less than 6:1, less than 5:1, less than 4:1, less than 3:1, or less than 2:1). In some aspects, the phospholipid content of the MBV comprises a phosphatidylcholine (PC) to phosphatidyl inositol (PI) ratio in the range of 0.5-1:1, or in the range of 1:0.5-1, or in the range of 0.5-1:2, or in the range of 2:0.5-1, or in the range of 0.8-1:1, or in the range of 1:0.8-1. In one aspect, the phospholipid content of the MBV comprises a phosphatidylcholine (PC) to phosphatidyl inositol (PT) ratio of about 1:1. In specific aspects, the phospholipid content of the MBV comprises a phosphatidylcholine (PC) to phosphatidyl inositol (PI) ratio of about 0.9:1.

In some aspects, the total phospholipid content of the MBV is 15%, 10%, 9%, 8%, 7%, 6%, 5%, 4% or less, or about 5%-10%, 5%-15%, 10%-15%, or 8%-12% of sphingomyelin (SM). In specific aspects, the total phospholipid content of the MBV is 10% or less of sphingomyelin (SM). In some aspects, the total phospholipid content of the is 15% or less of sphingomyelin (SM), 14% or less of sphingomyelin, 13% or less of sphingomyelin, 12% or less of sphingomyelin, 11% or less of sphingomyelin, 10% or less of sphingomyelin, 9% or less of sphingomyelin, 8% or less of sphingomyelin, 7% or less of sphingomyelin, 6% or less of sphingomyelin, 5% or less of sphingomyelin, or 4% or less of sphingomyelin.

In some aspects, the total phospholipid content of the MBV 20%, 19%, 18%, 17%, 16%, 15%, 14%, 13%, 12%, 11%, or 10% or less, or about 10%-20%, 15%-20%, 14%-18%, or 12%-16% of phosphatidylethanolamine (PE). In specific aspects, the total phospholipid content of the MBV is 20% or less of phosphatidylethanolamine (PE).

In some aspects, the total phospholipid content of the MBV is 5%, 10%, 12%, 15%, 18%, 20%, 25%, or 30% or greater, or about 5%-30%, 10%-20%, 10-25%, 15%-25%, or 12%-18% of phosphatidylinositol (PI). In specific aspects, MBV include a phospholipid content 15% or greater of phosphatidylinositol (PI).

In specific aspects, the total phospholipid content of the MBV comprises 15% or more phosphatidylinositol, 20% or less phosphatidylethanolamine, and 10% or less sphingomyelin. In specific aspects, the total phospholipid content of the MBV is 15% or more phosphatidylinositol and 20% or less phosphatidylethanolamine. In specific aspects, the total phospholipid content of the MBV is 15% or more phosphatidylinositol and 10% or less sphingomyelin. In specific aspects, the total phospholipid content of the MBV comprises 20% or less phosphatidylethanolamine and 10% or less sphingomyelin. In specific aspects, the total phospholipid content of the MBV is more than 15% phosphatidylinositol, 20% or less phosphatidylethanolamine, 10% or less sphingomyelin, and at least 55% of phosphatidylinositol and phosphatidylcholine in combination. In one aspect, the total phospholipid content of the MBV is at least 55% phosphatidylcholine (PC) and phosphatidyl inositol (PI) in combination and 10% or less sphingomyelin (SM). In specific aspects, the total phospholipid content of the MBV is at least 55% of phosphatidylinositol and phosphatidylcholine in combination and more than 15% phosphatidylinositol. In specific aspects, the total phospholipid content of the MBV is 55% of phosphatidylinositol and phosphatidylcholine in combination and 20% or less phosphatidylethanolamine.

The MBV may also comprise lysyl oxidase (Lox). Generally, nanovesicles derived from the ECM have a higher Lox content than exosomes. Lox is expressed on the surface of MBV. Nano-LC MS/MS proteomic analysis can be used to detect Lox proteins. Quantification of Lox can be performed (see, e.g., Hill R C, et al., Mol Cell Proteomics. 2015; 14(4):961-73, incorporated herein by reference in its entirety).

In some aspects, MBV are characterized by one or more of the following features:
(i) do not contain detectable levels of alkaline phosphatase;
(ii) do not contain detectable levels of osteopontin;
(iii) do not contain detectable levels of osteoprogeterin;
(iv) do not contain detectable levels of complement C5; and/or
(v) do not contain detectable levels of c-reactive protein.

In some aspects, MBV are characterized by one or more of the following features:
(i) contain low or do not contain detectable levels of EpCAM,
(ii) contain low or do not contain detectable levels of ANXA5,
(iii) contain low or do not contain detectable levels of TSG101;
(iv) contain low or do not contain detectable levels of FLOT1;
(v) contain low or do not contain detectable levels of ICAM1;
(vi) contain low or do not contain detectable levels of GM130; and/or
(vii) contain low or do not contain detectable levels of ALIX.

In one embodiment, MBV are characterized by low or undetectable levels of ANXA5, TSG101, and ICAM1.

In one embodiment, MBV are characterized by low or undetectable levels of CD81, CD63, ANXA5, TSG101, and ICAM1.

In certain aspects, the MBV comprise one or more miRNA. In specific non-limiting examples, the MBV comprise one, two, or all three of miR-143, miR-145 and miR-181. MiR-143, miR-145 and miR-181 are known in the art.

The miR-145 nucleic acid sequence is provided in MiRbase Accession No. M10000461, incorporated herein by reference. A miR-145 nucleic acid sequence is CACCUUGUCCUCACGGUCCAGUUUUCCCAGGAAUCCCUUAGAUGCUAAGAUGGGGAUUCCU GGAAAUACUGUUCUUGAGGUCAUGGUU (SEQ ID NO: 1). An miR-181 nucleic acid sequence is provided in miRbase Accession No. MI0000269, incorporated herein by reference. A miR-181 nucleic acid sequence is:

(SEQ ID NO: 2)
AGAAGGGCUAUCAGGCCAGCCUUCAGAGGACUCCAAGGAAC
AUUCAACGCUGUCGGUGAGUUUGGGAUUUGAAAAAACCACU
GACCGUUGACUGUACCUUGGGGUCCUUA.

The miR-143 nucleic acid sequence is provided in NCBI Accession No. NR_029684.1, Mar. 30, 2018, incorporated herein by reference. A DNA encoding an miR-143 nucleic acid sequence is: GCGCAGCGCC CTGTCTCCCA GCCTGAGGTG CAGTGCTGCA TCTCTGGTCA GTTGGGAGTC TGAGATGAAG CACTGTAGCT CAGGAAGAGA GAAGTTGTTC TGCAGC (SEQ ID NO: 3).

Following administration, the MBV maintain expression of F4/80 (a macrophage marker) and CD-11b on macrophages in the subject. Nanovesicle treated macrophages are predominantly F4/80+Fizz1+indicating an M2 phenotype.

The MBV disclosed herein can be formulated into compositions for pharmaceutical delivery. MBV are further disclosed and described in PCT Publication No. WO 2017/151862, which is incorporated herein by reference.

### Isolation of MBV from ECM

MBV can be produced from ECM that is produced by any cells of interest, or they be isolated from a commercial source of ECM, as described supra. The MBV can be produced from the same species as, or a different species than, the subject being treated. In some aspects, these methods include digesting the ECM with an enzyme to produce digested ECM. In specific aspects, the ECM is digested with one or more of pepsin, elastase, hyaluronidase, collagenase a metalloproteinase, and/or proteinase K, or combinations thereof. In a specific non-limiting example, the ECM is digested with only elastase and/or a metalloproteinase. In another non-limiting example, the ECM is not digested with collagenase and/or trypsin and/or proteinase K. In other aspects, the ECM is treated with a detergent. In further aspects, the method does not include the use of enzymes. In specific non-limiting examples, the method utilizes chaotropic agents or ionic strength to isolate MBV such as salts, such as potassium chloride. In additional aspects, the ECM can be manipulated to increase MBV content prior to isolation of MBV. Techniques for isolating MBV from ECM are described, for example, in U.S. Patent Application Publication No. 2019/0117837, the contents of which are incorporated by reference herein for all purposes. Techniques for isolating MBV are also disclosed in Quijano et al., Tissue Eng Part C Methods. 2020 October; 26(10):528-540, also incorporated by reference herein.

In some aspects, the ECM is digested with an enzyme. The ECM can be digested with the enzyme for about 12 to about 48 hours, such as about 12 to about 36 hours. The ECM can be digested with the enzyme for about 12, about 24 about 36 or about 48 hours. In one specific non-limiting example, the ECM is digested with the enzyme at room temperature. However, the digestion can occur at about 4 °C, or any temperature between about 4 °C and 25 °C Generally, the ECM is digested with the enzyme for any length of time, and at any temperature, sufficient to remove collagen fibrils. The digestion process can be varied depending on the tissue source. Optionally, the ECM is processed by freezing and thawing, either before or after digestion with the enzyme. The ECM can be treated with detergents, including ionic and/or non-ionic detergents.

The digested ECM is then processed, such as by centrifugation, to isolate a fibril-free supernatant. In some aspects the digested ECM is centrifuged, for example, for a first step at about 300 to about 1000 g. Thus, the digested ECM can be centrifuged at about 400 g to about 750 g, such as at about 400 g, about 450 g, about 500 g or about 600 g. This centrifugation can occur for about 10 to about 15 minutes, such as for about 10 to about 12 minutes, such as for about 10, about 11, about 12, about 14, about 14, or about 15 minutes. The supernatant including the digested ECM is collected.

In some aspects, the MBV comprise Lox. In some aspects, methods for isolating such MBV include digesting the extracellular matrix with elastase and/or metalloproteinase to produce digested extracellular matrix, centrifuging the digested extracellular matrix to remove collagen fibril remnants and thus to produce a fibril-free supernatant, centrifuging the fibril-free supernatant to isolate the solid materials, and suspending the solid materials in a carrier.

In some aspects, digested ECM also can be centrifuged for a second step at about 2000 g to about 3000 g. Thus, the digested ECM can be centrifuged at about 2,500 g to about 3,000 g, such as at about 2,000 g, 2,500 g, 2,750 g or 3,000 g. This centrifugation can occur for about 20 to about 30 minutes, such as for about 20 to about 25 minutes, such as for about 20, about 21, about 22, about 23, about 24, about 25, about 26, about 27, about 28, about 29 or about 30 minutes. The supernatant including the digested ECM is collected.

In additional aspects, the digested ECM can be centrifuged for a third step at about 10,000 to about 15,000 g. Thus, the digested ECM can be centrifuged at about 10,000 g to about 12,500 g, such as at about 10,000 g, 11,000 g or 12,000 g. This centrifugation can occur for about 25 to about 40 minutes, such as for about 25 to about 30 minutes, for example for about 25, about 26, about 27, about 28, about 29, about 30, about 31, about 32, about 33, about 34, about 35, about 36, about 37, about 38, about 39 or about 40 minutes. The supernatant including the digested ECM is collected. One, two or all three of these centrifugation steps can be independently utilized. In some aspects, all three centrifugation steps are utilized. The centrifugation steps can be repeated, such as 2, 3, 4, or 5 times. In one aspect, all three centrifugation steps are repeated three times.

In some aspects, the digested ECM is centrifuged at about 500 g for about 10 minutes, centrifuged at about 2,500 g for about 20 minutes, and/or centrifuged at about 10,000 g for about 30 minutes. These step(s), such as all three steps are repeated 2, 3, 4, or 5 times, such as three times. Thus, in one non-limiting example, the digested ECM is centrifuged at about 500 g for about 10 minutes, centrifuged at about 2,500 g for about 20 minutes, and centrifuged at about 10,000 g for about 30 minutes. These three steps are repeated three times. Thus, a fibril-free supernatant is produced. The fibril-free supernatant is then centrifuged to isolate the MBV. In some aspects, the fibril-free supernatant is centrifuged at about 100,000 g to about 150,000 g. Thus, the fibril-free supernatant is centrifuged at about 100,000 g to about 125,000 g, such as at about 100,000 g, about 105,000 g, about 110,000 g, about 115,000 g or about 120,000 g. This centrifugation can occur for about 60 to about 90 minutes, such as about 70 to about 80 minutes, for example for about 60, about 65, about 70, about 75, about 80, about 85 or about 90 minutes. In one non-limiting example, the fiber-free supernatant is centrifuged at about 100,000 g for about 70 minutes. The solid material is collected, which is the MBV. These MBV then can be re-suspended in any carrier of interest, such as, but not limited to, a buffer.

In further aspects the ECM is not digested with an enzyme. In these methods, ECM is suspended in an isotonic saline solution, such as phosphate buffered saline. Salt is then added to the suspension so that the final concentration of the salt is greater than about 0.1 M. The concentration can be, for example, up to about 3 M, for example, about 0.1 M salt to about 3 M, or about 0.1 M to about 2M. The salt can be, for example, about 0.1M, 0.15M, 0.2M, 0.3M, 0.4 M, 0.7 M, 0.6 M, 0.7 M, 0.8M., 0.9M, 1.0 M, 1.1 M, 1.2 M, 1.3 M, 1.4 M, 1.5M, 1.6 M, 1.7 M, 1.8M, 1.9 M, or 2M. In some non-limiting examples, the salt is potassium chloride, sodium chloride or magnesium chloride. In other aspects, the salt is sodium chloride, magnesium chloride, sodium iodide, sodium thiocyanate, a sodium salt, a lithium salt, a cesium salt or a calcium salt.

In some aspects, the ECM is suspended in the salt solution for about 10 minutes to about 2 hours, such as about 15 minutes to about 1 hour, about 30 minutes to about 1 hour, or about 45 minutes to about 1 hour. The ECM can be suspended in the salt solution for about 15, 20, 25, 30, 35, 40, 45, 50, 55, 60, 65, 70, 75, 80, 85, 90, 95, 100, 105, 110, 115 or 120 minutes. The ECM can be suspended in the salt solution at temperatures from 4 °C to about 50 °C, such as, but not limited to about 4 °C to about 25 °C or about 4 °C to about 37 °C In a specific non-limiting example, the ECM is suspended in the salt solution at about 4 °C In other specific non-limiting examples, the ECM is suspended in the salt solution at about 22 °C or about 25 °C (room temperature). In further non-limiting examples, the ECM is suspended in the salt solution at about 37 °C.

In some aspects, the method includes incubating an extracellular matrix at a salt concentration of greater than about 0.4 M; centrifuging the digested extracellular matrix to remove collagen fibril remnants, and isolating the supernatant; centrifuging the supernatant to isolate the solid materials; and suspending the solid materials in a carrier, thereby isolating MBV from the extracellular matrix.

Following incubation in the salt solution, the ECM is centrifuged to remove collagen fibrils. In some aspects, digested ECM also can be centrifuged at about 2000 g to about 5000 g. Thus, the digested ECM can be centrifuged at about 2,500 g to about 4,500 g, such as at about 2,500 g, about 3,000 g, 3,500, about 4,000 g, or about 4,500 g. In one specific non-limiting example, the centrifugation is at about 3,500 g. This centrifugation can occur for about 20 to about 40 minutes, such as for about 25 to about 35 minutes, such as for about 20, about 21, about 22, about 23, about 24, about 25, about 26, about 27, about 28, about 29, about 30 minutes, about 31, about 32, about 33 about 34 or about 35 minutes. The supernatant is then collected.

In additional aspects, the supernatant then can be centrifuged for a third step at about 100,000 to about 150,000 g. Thus, the digested ECM can be centrifuged at about 100,000 g to about 125,000 g, such as at about 100,000 g, 110,000 g or 120,000 g. This centrifugation can occur for about 30 minutes to about 2.5 hour, such as for about 1 hour to about 3 hours, for example for about 30 minutes, about 45 minutes, about 60 minutes, about 90 minutes, or about 120 minutes (2 hours). The solid materials are collected and suspended in a solution, such as buffered saline, thereby isolating the MBV.

In yet other aspects, the ECM is suspended in an isotonic buffered salt solution, such as, but not limited to, phosphate buffered saline. Centrifugation or other methods can be used to remove large particles (see below). Ultrafiltration is then utilized to isolate MBV from the ECM, particles between about 10 nm and about 10,000 nm, such as between about 10 and about 1,000 nm, such as between about 10 nm and about 300 nm.

In specific non-limiting examples, the isotonic buffered saline solution has a total salt concentration of about 0.164 mM, and a pH of about 7.2 to about 7.4. In some aspects, the isotonic buffered saline solution includes 0.002 M KCl to about 0.164 M KCL, such as about 0.0027 M KCl (the concentration of KCL in phosphate buffered saline). This suspension is then processed by ultracentrifugation.

Following incubation in the isotonic buffered salt solution, the ECM is centrifuged to remove collagen fibrils. In some aspects, digested ECM also can be centrifuged at about 2000 g to about 5000 g. Thus, the digested ECM can be centrifuged at about 2,500 g to about 4,500 g, such as at about 2,500 g, about 3,000 g, 3,500, about 4,000 g, or about 4,500 g. In one specific non-limiting example, the centrifugation is at about 3,500 g. This centrifugation can occur for about 20 to about 40 minutes, such as for about 25 to about 35 minutes, such as for about 20, about 21, about 22, about 23, about 24, about 25, about 26, about 27, about 28, about 29, about 30 minutes, about 31, about 32, about 33 about 34 or about 35 minutes.

Microfiltration and centrifugation can be used and combined to remove large molecular weight materials from the suspension. In one aspect, large size molecule materials, such as more than 200 nm are removed using microfiltration. In another aspect, large size materials are removed by the use of centrifugation. In a third aspect both microfiltration and ultracentrifugation are used to remove large molecular weight materials. Large molecular weight materials are removed from the suspended ECM, such as materials greater than about 10,000 nm, greater than about 1,000 nm, greater than about 500 nm, or greater than about 300 nm.

The effluent for microfiltration or the supernatant is then subjected to ultrafiltration. Thus, the effluent, which includes particle of less than about 10,000 nm, less than about 1,000 nm, less than about 500 nm, or less than about 300 nm is collected and utilized. This effluent is then subjected to ultrafiltration with a membrane with a molecular weight cutoff (MWCO) of 3,000 to 100,000.

### Preparation of Extracellular Matrix (ECM) and Hydrogels

Any type of extracellular matrix can be used to produce a mammalian ECM hydrogel (see U.S. Pat. Nos. 4,902,508; 4,956,178; 5,281,422; 5,352,463; 5,372,821; 5,554,389; 5,573,784; 5,645,860; 5,771,969; 5,753,267; 5,762,966; 5,866,414; 6,099,567; 6,485,723; 6,576,265; 6,579,538; 6,696,270; 6,783,776; 6,793,939; 6,849,273; 6,852,339; 6,861,074; 6,887,495; 6,890,562; 6,890,563; 6,890,564; and 6,893,666 related to ECM). In certain aspects, the ECM is isolated from a vertebrate animal, for example and without limitation, from a mammal including, but not limited to, humans, monkeys, horses, pigs, cows and sheep. In specific non-limiting examples, the ECM is porcine.

ECM can be derived from any organ or tissue, including without limitation, urinary bladder, intestine (such as small intestine or large intestine), heart, kidney, uterus, brain, blood vessel, lung, bone, muscle, pancreas, stomach, spleen adipose tissue, liver, esophagus and dermis. The ECM can be obtained from a cell culture. In one aspect, the ECM is isolated from a urinary bladder. In another aspect, the ECM is from an esophagus. In another aspect, the ECM is from dermis. In another aspect, the ECM is from small intestinal submucosa (SIS). The ECM may or may not include the basement membrane portion of the ECM. In certain aspects, the ECM includes at least a portion of the basement membrane. A tissue can be decellularized to remove cells and cellular material, e.g., from the source tissue or organ, to produce an ECM. It desirable to use a decellularized material prevent an immune response, such as when ECM is implanted in a subject, for example, as a component of a hydrogel disclosed herein. Removal of cellular material, such as when using ECM to form a hydrogel, prevents such an immune response.

U.S. Pat. No. 8,361,503 (incorporated herein by reference in its entirety for all purposes) discloses preparation of a urinary bladder ECM, such as porcine bladder ECM is prepared by abrading bladder tissue to remove the outer layers including both the tunica serosa and the tunica muscularis using a longitudinal wiping motion with a scalpel handle and moistened gauze. Following eversion of the tissue segment, the luminal portion of the tunica mucosa is delaminated from the underlying tissue using the same wiping motion. In some aspects, perforation of the submucosa is prevented. After these tissues are removed, the resulting ECM consists mainly of the tunica submucosa.

The production of hydrogels from dermal ECM is disclosed in Wolf et al., Biomaterials 33: 7028-7038, 2012, incorporated herein by reference. The production of ECM from esophageal tissue is disclosed, for example, in Badylak et al. J Pediatr Surg. 35(7):1097-103, 2000 and Badylak et al., J Surg Res. 2005 September; 128(1):87-97, 2005, both incorporated herein by reference. U.S. Pat. No. 6,893,666, incorporated herein by reference, discloses production of ECM from urinary bladder, skin, esophagus and small intestine. ECM can be produced from any of these tissues.

Commercially available ECM preparations can also be used. In one aspect, the ECM is derived from small intestinal submucosa or SIS. Commercially available preparations include, but are not limited to, SURGISIS™, SURGISIS-ES™, STRATASIS™, and STRATASIS-ES™ (Cook Urological Inc.; Indianapolis, Ind.) and GRAFTPATCH™ (Organogenesis Inc.; Canton Mass.). In another aspect, the ECM is derived from dermis. Commercially available preparations include, but are not limited to PELVICOL™ (sold as PERMACOL™ in Europe; Bard, Covington, Ga.), REPLIFORM™ (Microvasive; Boston, Mass.) and ALLODERM™ (LifeCell; Branchburg, N.J.). In another aspect, the ECM is derived from urinary bladder. Commercially available preparations include, but are not limited to, UBM (Acell Corporation; Jessup, Md.).

Tissue for preparation of ECM can be harvested in a large variety of ways and once harvested, a variety of portions of the harvested tissue may be used. ECM has also been prepared from the esophagus and small intestine, see, for example, Keane et al., Tissue Eng. Part A, 21(17-18): 2293-2300, 2015, incorporated herein by reference. Esophageal ECM can be prepared by mechanically separating the mucosa and submucosa from the muscularis externa and digesting the mucosal layers in a buffer including trypsin, followed by exposure to sucrose, TRITON-X100®, deoxycholic acid, peracetic acid and DNAse. Small intestine submucosa (SIS) can be prepared by mechanically removing the superficial layers of the tunica mucosa, tunica serosa, and tunica muscularis externa from the intact small intestine, leaving the submucosa, muscularis mucosa, and basilar stratum compactum intact. The SIS is then treated with peracetic acid. Exemplary protocols are provided in Keane et al. Dermal hydrogels can be produced, for example, as disclosed in Wolf et al, J Biomed Mater Res A. 2013. 35(25):6838-49. PMID: 23873846. PMCID: 3808505, incorporated herein by reference.

In one aspect, the ECM is isolated from harvested porcine urinary bladder to prepare urinary bladder matrix (UBM). Excess connective tissue and residual urine are removed from the urinary bladder. The tunica serosa, tunica muscularis externa, tunica submucosa and most of the muscularis mucosa can be removed by mechanical abrasion or by a combination of enzymatic treatment, hydration, and abrasion. Mechanical removal of these tissues can be accomplished by abrasion using a longitudinal wiping motion to remove the outer layers (particularly the abluminal smooth muscle layers) and even the luminal portions of the tunica mucosa (epithelial layers). Mechanical removal of these tissues is accomplished by removal of mesenteric tissues with, for example, Adson-Brown forceps and Metzenbaum scissors and wiping away the tunica muscularis and tunica submucosa using a longitudinal wiping motion with a scalpel handle or other rigid object wrapped in moistened gauze. The epithelial cells of the tunica mucosa can also be dissociated by soaking the tissue in a de-epithelializing solution, for example and without limitation, hypertonic saline. The resulting UBM comprises basement membrane of the tunica mucosa and the adjacent tunica propria, which is further treated with peracetic acid, lyophilized and powdered, see U.S. Pat. No. 8,361,503, incorporated herein by reference.

Dermis sections can used for the preparation of the ECM hydrogels, see PCT Application No. 2015/15164728, incorporated herein by reference. In a specific non-limiting example, the dermis can be decellularized with 0.25% Trypsin/1% TRITON-X®-100 (i.e. no SDS) on a vortex shaker at 300 RPM at room temperature in the following solutions: 0.25% trypsin for 6 hours, 1×; deionized water, 15 minutes, 3×; 70% ethanol, 10 to 12 hours, 1×; 3% H₂O₂, 15 minutes, 1×, deionized water, 15 minutes, 2×; 1% TRITON-X®-100 in 0.26% EDTA/0.69% Tris, 6 hours, 1× and then overnight, 1×; deionized water, 15 minutes, 3×; 0.1% peracetic acid/4% ethanol, 2 hours, 1×; PBS, 15 minutes, 2×; and finally deionized water, 15 minutes, 2×. Dermis sheets are then lyophilized and subsequently reduced to particulate form using a Waring blender and a Wiley Mill with a #20 mesh screen.

In some aspects, the epithelial cells can be delaminated first by first soaking the tissue in a de-epithelializing solution such as hypertonic saline, for example and without limitation, 1.0 N saline, for periods of time ranging from 10 minutes to 4 hours. Exposure to hypertonic saline solution effectively removes the epithelial cells from the underlying basement membrane. The tissue remaining after the initial delamination procedure includes epithelial basement membrane and the tissue layers abluminal to the epithelial basement membrane. This tissue is next subjected to further treatment to remove the majority of abluminal tissues but not the epithelial basement membrane. The outer serosal, adventitial, smooth muscle tissues, tunica submucosa and most of the muscularis mucosa are removed from the remaining deepithelialized tissue by mechanical abrasion or by a combination of enzymatic treatment, hydration, and abrasion.

In some aspects, the ECM itself can be sterilized by any number of standard techniques, including, but not limited to, exposure to peracetic acid, low dose gamma radiation, gas plasma sterilization, ethylene oxide treatment or electron beam treatment. More typically, sterilization of ECM is obtained by soaking in 0.1% (v/v) peracetic acid, 4% (v/v) ethanol, and 95.9% (v/v) sterile water for two hours. The peracetic acid residue is removed by washing twice for 15 minutes with PBS (pH=7.4) and twice for 15 minutes with sterile water. ECM material can be sterilized by propylene oxide or ethylene oxide treatment, gamma irradiation treatment (0.05 to 4 mRad), gas plasma sterilization, peracetic acid sterilization, or electron beam treatment. The ECM can also be sterilized by treatment with glutaraldehyde, which causes cross linking of the protein material, but this treatment substantially alters the material such that it is slowly resorbed or not resorbed at all and incites a different type of host remodeling which more closely resembles scar tissue formation or encapsulation rather than constructive remodeling. Cross-linking of the protein material can also be induced with carbodiimide or dehydrothermal or photooxidation methods. As disclosed in U.S. Pat. No. 8,361,503, ECM is disinfected by immersion in 0.1% (v/v) peracetic acid (a), 4% (v/v) ethanol, and 96% (v/v) sterile water for 2 h. The ECM material is then washed twice for 15 min with PBS (pH=7.4) and twice for 15 min with deionized water.

Generally, following isolation of the tissue of interest, decellularization is performed by various methods, for example and without limitation, exposure to hypertonic saline, peracetic acid, TRITON-X® or other detergents. Sterilization and decellularization can be simultaneous. For example and without limitation, sterilization with peracetic acid, described above, also can be used for decellularization. ECM can then be dried, either lyophilized (freeze-dried) or air dried. Dried ECM can be comminuted by methods including, but not limited to, tearing, milling, cutting, grinding, and shearing. The comminuted ECM can also be further processed into a powdered form by methods, for example and without limitation, such as grinding or milling in a frozen or freeze-dried state.

Mammalian ECM is also commercially available. These include AVITENE™, MICROMATRIX® and XENMATRIX™. These commercially available products can also be used to produce a mammalian acoustic ECM hydrogel.

### Preparation of Acoustic ECM Hydrogels

In some aspects, a comminuted ECM, such as a mammalian ECM, is diluted in a liquid for preparation of an acoustic ECM hydrogel. The ECM may or may not be lyophilized prior to comminuting. The ECM can be comminuted, for example, by grinding, chopping or cutting the ECM. Comminuted ECM should have pieces in the range of about 10 μm to about 5000 μm, about 10 μm to about 4000 μm, about 10 μm to about 3000 μm, about 10 μm to about 2000 μm, about 10 μm to about 1000 μm, about 10 μm to about 500 μm, about 30 μm to about 300 μm, about 40 to about 400 μm, about 25 μm to about 500 μm, about 50 μm to about 500 μm, about 100 μm to about 300 μm, about 10 μm to about 50 μm, or about 10 μm to about 100 μm. In one aspect, the ECM is provided in pieces having a range from about 10 μm to about 1000 μm. In another preferred aspect, the ECM is provided in pieces having a range from about 10 μm to about 2000 μm. In one non-limiting example, the pieces are in the range of about 30 μm to about 300 μm. The liquid can be a buffer at neutral pH, such as, for example, a pH of about 7.0 to about 7.6, such as about 7.1 to about 7.5, such as about 7.2 to about 7.4, such as about 7.0 to 7.2, such as about 7.0 to 7.4, such as about 7.1, 7.2, 7.3, 7.4, 7.5 or 7.6. The ECM can be diluted in an isotonic buffered saline solution, such as, but not limited to, phosphate buffered saline (PBS) or Tris buffered saline. In some aspects, the buffered saline solution has an osmolarity of about 290 mOsm/L. The liquid can be water. In some aspects, the isotonic buffer, including, without limitation, Phosphate Buffered Saline (PBS), can be used to bring the solution to a target pH, or to aid in maintaining the pH and ionic strength of the gel to target levels, such as physiological pH and ionic conditions. This forms a liquid ECM solution.

The methods for preparation of an acoustic hydrogel generally do not involve the use of an acid protease, including pepsin, trypsin, or hyaluronidase, or enzymatic digestion of the ECM tissue, generally. See PCT Application No. WO 2015/164728, incorporated herein by reference. Generally, the solubilized ECM in the liquid is not contacted with an acid protease.

Methods of preparing an extracellular matrix hydrogel by application of acoustic techniques, e.g., ultrasonic frequencies, may be found in U.S. Patent Application Publication No. 2022/0143265, the contents of which is incorporated by reference herein for all purposes. In some aspects, the ECM is utilized at a concentration of greater than about 25 mg/ml in the liquid. The ECM can be utilized at a concentration of about 25 mg/ml to about 600 mg/ml in the liquid, such as the buffer. Suitable concentrations also include about 25 mg/ml to about 300 mg/ml, about 25 mg/ml to about 200 mg/ml, and about 25 mg/ml to about 150 mg/mi. The ECM can be utilized at a concentration of about 50 mg/ml to 600 mg/ml in the liquid, such as the buffer. Suitable concentrations also include about 50 mg/ml to about 300 mg/ml, about 50 mg/ml to about 200 mg/ml, and about 50 mg/ml to about 150 mg/ml. Suitable concentrations include about 25, 30, 35, 40, 45, 50, 55, 60, 65, 70, 75, 80, 85, 90, 95, 100, 105, 110, 115, 120, 125, 130, 135, 140, 150, 155, 160, 165, 170, 175, 180, 185, 190, 195, and 200 mg/ml. Exemplary concentrations include about 25 mg/ml, 100 mg/ml, and 150 mg/ml. In one non-limiting example, the ECM in a liquid at a concentration of about 25 mg/nil to about 150 mg/ml. In one non-limiting example, the ECM is in the liquid at a concentration of 100 mg/ml.

The ECM in the liquid, such as the buffered saline solution, is treated with an ultrasound frequency. In one aspect, the ultrasound is at a frequency of about 20 kHz to about 100 kHz. The ECM in the liquid can be treated with ultrasound at a frequency of about 20 kHz to about 30 kHz, about 20 Hz to about 40 kHz, about 20 kHz to about 50 kHz, about 20 kHz to about 60 kHz, about 20 kHz to about 70 kHz, about 20 kHz to about 80 kHz, or about 20 kHz to about 90 kHz. The ECM in the liquid can be treated with ultrasound at a frequency of about 20 kHz, 30 kHz, 40 kHz, 50 kHz, 60 kHz, 70 kHz, 80 kHz, 90 kHz or 100 kHz. In one non-limiting example, the ECM in the liquid can be treated with ultrasound at a frequency of about 20 kHz.

The ECM in the liquid, such as the buffered saline solution, is treated with ultrasound for at least 20 seconds, such as at least 30 seconds. The ECM in the liquid, such as the buffered saline solution, is treated with ultrasound for at least 60 seconds. In some aspects, the ECM in the liquid is treated with ultrasound for at least 60 seconds to about one hour. In further aspects, the ECM in the liquid is treated with ultrasound for at least 60 seconds to about 30 minutes. In further aspects, the ECM in the liquid is treated with ultrasound for at least 30 seconds to about 30 minutes. In more aspects, the ECM in the liquid is treated with ultrasound for at least 60 seconds to about 15 minutes. In more aspects, the ECM in the liquid is treated with ultrasound for at least 30 seconds to about 15 minutes. In some aspects, the ECM in the liquid is treated with ultrasound for at least 60 seconds to about 10 minutes. In some aspects, the ECM in the liquid is treated with ultrasound for at least 30 seconds to about 10 minutes. In some aspects, the ECM in the liquid is treated with ultrasound for at least 60 seconds to about 5 minutes. In some aspects, the ECM in the liquid is treated with ultrasound for at least 30 seconds to about 5 minutes. The ECM in the liquid can treated with ultrasound for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 minutes. In some aspects, the ECM in the liquid is treated with the ultrasound in pulses for a total time as listed herein. Thus, in some aspects, the ECM in the liquid, such as the buffered saline solution, is treated with pulses, such as of at least about 30 seconds in length, such as about 30, about 40 or about 60 seconds in length. The ECM in the liquid such as the buffered saline solution, can be treated 1, 2, 3, 4, 5, 6, 7, 8, 9, 10 times, with the ultrasound, such that the total time of treatment is the 60 seconds to one hour, or any of the total times listed. The ECM in the liquid such as saline solution can be treated for 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 seconds. The ECM in the liquid such as saline solution can be treated for at least 30 seconds. Generally, if multiple treatments are used, they occur in a period of less than 1 hour. An exemplary method is pulses of 30 seconds of ultrasound, followed by no treatment for 30 to 45 seconds, followed by another treatment. This treatment is applied 2, 3, 4, 5, 6, 7, 8, 9 or 10 or more times. One exemplary non-limiting method is six pulses of 30 seconds of ultrasound, such as at about 20 kHz, followed by 45 seconds off, for six repetitions, totaling 3 minutes of treatment with ultrasound.

The ultrasound can have an amplitude of about 20 μm to about 320 μm. Generally, the amplitude is measure from the center of the probe used to produce the ultrasound. The amplitude of the probe's vibrating surface the distance between its position in the probe's fully extended and fully contracted states, measured in microns (μm). In some aspects, the amplitude is about 30 μm to about 200 μm. In further aspects, the amplitude is about 36 μm to about 180 μm. The amplitude can be about 30, 40, 50, 60, 70, 80, 90, 100, 110, 120, 130, 140, 150, 150, 160, 70, 180, 190, 200, 210, 220, 230, 240, 250, 260, 270, 280, 290 or 300 μm. In some aspects, the amplitude can be about 30-40 μm, 40-50 μm, 50-60 m, 60-70 μm, 70-80 μm, 80-90 μm, 90-100 μm, 100-110, 110-120 μm, 120-130 μm, 130-140 μm, 140-150 μm, 150-160 μm, 160-170 μm, 170-180 μm, 180-190 μm, 190-200 μm, 200-210 μm, 210-220 μm 220-230 μm, 230-240 μm, 240-250 μm, 250-260 μm, 260-270 μm, 270-280 μm, 280-290 μm or 290-300 μm. In one specific, non-limiting example, the ultrasound is at a frequency of about 20 kHz, and the amplitude is about 36 μm to about 180 μm. In a further non-limiting example, the ultrasound is at a frequency of about 20 kHz, and the amplitude is about 36 μm to about 180 μm, and the treatment is for a total of about 1, 2, 3, 4, or 5 minutes, such as about 3 minutes. The sonication can be for about 1, 2, 3, 4, 5, 6, 7, 8, 9, 10, 11, 12, 13, 14, 15, 16, 17, 18, 19 or 20 minutes. The sonication can be from about 30 seconds to about 5 minutes. The sonication can be for example, for between about 1 to about 5 minutes. The sonication can be for about 1 to about 10 minutes. The sonication can be, for example, for between 1 to about 20 minutes. In more aspects, the sonication can be for less than about one hour, less than about 30 minutes, less than about 20 minutes, or less than about 10 minutes. In some aspects, the sonication can be for at least 30 seconds. In other aspects, the sonication can be for about 10 minutes to about 24 hours, for example, about 1, 2, 3, 4, 5, 6, 7, 8, 9, 10, 11, 12, 13, 14, 15, 16, 17, 18, 19, 20, 21, 22, 23, or 24 hours. In some aspects, sonication can be for up to 48 hours.

In some aspects, the ECM in the liquid is treated with the ultrasound at a temperature in a range of about 30 °C to about 43 °C In one aspect, the ECM in the liquid is treated with the ultrasound at a temperature in the range of about 35 °C to about 40 °C In one aspect, the ECM in the liquid is treated with ultrasound at a temperature in the range of about 36 °C to about 38 °C In another aspect, the ECM in the liquid is treated with ultrasound at a temperature in the range of about 37 °C or greater, such as a temperature of about 37 °C to about 55 °C, such as about 37 °C to about 50 °C, such as about 37 °C to about 45 °C, such as about 37 °C to about 40 °C The ECM in the liquid is treated with the ultrasound at a temperature of about 37, 38, 39, 40, 41, 42, 43, 44, 45, 46, 47, 48, 49, 50, 51, 52, 53, 54, or 55 °C In further aspects, the ECM in the liquid is treated with the ultrasound at greater than about 38 °C, such as about 38 °C to about 50 °C, such as about 38 °C to about 45 °C, such as about 38 °C to about 40 °C.

In aspects, treatment with ultrasound produces an acoustic ECM hydrogel. The acoustic ECM hydrogel generally experiences a phase transition from sol to gel around 37 °C and therefore transitions to a liquid phase at greater than 37 °C, and to a gel phase at below 37 °C. At 37 °C the acoustic ECM hydrogel is sufficiently viscous to resemble a gel; however, as the temperature is increased above 37 °C, the gel transitions to a sol. The acoustic ECM hydrogel forms a gel (sol to gel transition) upon a decrease in temperature below 37 °C Thus, in some aspects, following sonication, the acoustic ECM hydrogel is cooled to a temperature of less than 37 °C, such as about 4 °C to about 36 °C The acoustic ECM hydrogel can be cooled to room temperature, which is generally about 25 °C In some aspects, the acoustic ECM hydrogel is cooled to about 15 °C to about 25 °C The acoustic ECM hydrogel can be cooled to about 23 °C to about 27 °C The acoustic ECM hydrogel can be cooled to about 4, 5, 6, 7, 8, 9, 10, 11, 12, 13, 14, 15, 16, 17, 18, 19, 20, 21, 22, 23, 24, 25, 26, 27, 28 29 or 30 °C to induce the gel phase.

In some aspects, exogenous MBV may be added to ECM solution prior to sonication. In yet other aspects, exogenous MBV may be added to the acoustic ECM hydrogel after sonication. The exogenous MBV may be added to the ECM hydrogel before the hydrogel transitions to a gel (e.g., while it is in a liquid phase); therefore, in one aspect, exogenous MBV are added to the acoustic ECM hydrogel at a temperature greater 37 °C to produce a composition comprising an acoustic hydrogel disclosed herein containing exogenous MBV. In another aspect, the exogenous MBV are added to the acoustic ECM hydrogel during its gel phase, e.g., at a temperature lower than 37 °C For example, disclosed herein in is an acoustic ECM hydrogel comprising exogenous MBV.

In some aspects, disclosed is an acoustic mammalian ECM hydrogel, wherein the hydrogel is thermoreversible, wherein the hydrogel is in a solid (gel) phase at temperatures below about 37 °C and is in a liquid (sol) phase at temperatures of greater than 37 °C The acoustic hydrogel can be produced using any of the methods disclosed herein. In some aspects, the storage modulus (G′) is greater than loss modulus (G″) by about an order of magnitude for the acoustic ECM hydrogel. In further aspects, wherein the viscosity of the acoustic ECM hydrogel decreases with increased stress at a temperature of about 15 to about 37 °C, such as at about 15, 15, 16, 17, 18, 20, 21, 22, 23, 24, 25, 26, 27, 28, 29, 30, 31, 32, 33, 34, 35, and/or 36 °C In further aspects, the viscosity of the acoustic ECM hydrogel decreases with increased stress at room temperature, and/or at about 23 °C to about 27 °C and/or about 15 °C to about 25 °C In one aspect, the gel to sol transition of the acoustic ECM hydrogel is at about 37 °C, such that the hydrogel can be used as a submucosal cushion because it is sufficiently viscous at body temperature.

These acoustic ECM hydrogels can be made from any mammalian ECM disclosed above. In specific, non-limiting example, the ECM is human ECM. In other non-limiting examples, the ECM is urinary bladder ECM, small intestinal submucosal ECM, esophageal EMC, or dermal ECM. In one aspect, the ECM is urinary bladder ECM. In another aspect, the ECM is dermal ECM. In yet another aspect, the ECM is esophageal ECM. The source of ECM may be, for example, porcine, bovine, or ovine.

In some aspects, the acoustic ECM hydrogel includes ECM at a concentration of about 25 mg/ml to about 600 mg/ml. In further aspects, the acoustic ECM hydrogel includes ECM at a concentration of about 20 mg/ml to about 600 mg/ml, about 25 mg/ml to about 300 mg/ml, about 25 mg/ml to about 200 mg/ml, and about 25 mg/ml to about 150 mg/ml. In more aspects, the acoustic ECM hydrogel includes ECM at a concentration of about 50 mg/ml to 600 mg/nil in the liquid, such as in the buffer. The acoustic ECM hydrogel also can have an ECM concentration of about 50 mg/ml to about 300 mg/ml, about 50 mg/ml to about 200 mg/nil, about 50 mg/nil to about 150 mg/ml, about 50-100 mg/ml, or about 100-150 mg/ml. In some non-limiting examples, the acoustic ECM hydrogel includes ECM at a concentration of about 20, 25, 30, 35, 40, 45, 50, 55, 60, 65, 70, 75, 80, 85, 90, 95, 100, 105, 110, 115, 120, 125, 130, 135, 140, 150, 155, 160, 165, 170, 175, 180, 185, 190, 195, and 200 mg/ml. In some non-limiting examples, the acoustic ECM hydrogel includes ECM at a concentration of about 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, and 195-200 mg/ml. Exemplary non-limiting concentrations of ECM also include about 25 mg/ml, 100 mg/ml, and 150 mg/ml. In one non-limiting example, the acoustic ECM hydrogel includes ECM at a concentration of about 25 mg/ml to about 150 mg/ml. In one aspect, the ECM concentration is about 100 mg/ml.

In some aspects, the acoustic ECM hydrogel has a viscosity of about 1400 Pa*s at 15 °C, and a viscosity of about 400 Pa*s at a temperature of 25 °C, when the concentration of ECM is about 150 mg/mL. In other aspects, the acoustic ECM hydrogel has a storage modulus of approximately 2700 Pa*s at 15 °C, approximately 800 Pa*s at 25 °C, and 600 Pa*s at 37 °C, when the concentration of ECM is about 150 mg/mL.

The acoustic ECM hydrogel in the liquid phase, can be placed into a three-dimensional cast prior to cooling, or spread on a TEFLON® sheet to form a film. The high concentration of ECM in (50 to 600 mg/ml) in the acoustic ECM hydrogel allows for the formation of very thin sheets, for example a sheet as thin as 4 microns. The acoustic ECM hydrogel can be configured to any size greater than 4 microns and in any 2-dimensional or 3-dimensional shape. In some aspects, a sheet is formed that is about 4 to about 10 microns in thickness, such as about 4, 5, 6, 7, 8, 9, or 10 microns in thickness. The acoustic ECM hydrogel can be formed into any three-dimensional shape, which includes, without limitation a cylinder, sphere, ellipsoid, disk, sheet, cube, cuboid, cone, triangular or rectangular prism, as well as hollow spheres, hollow ellipsoids, and open-ended hollow cylinders, etc. The acoustic ECM hydrogel can also be used as an injectable, such as by placing it in a syringe and extruding it from the syringe in either a gel or sol phase.

In some aspects, the mammalian acoustic ECM hydrogel includes solubilized ECM at a concentration of greater than about 0.1 mg/ml. The mammalian acoustic ECM hydrogel can include solubilized ECM at a concentration of about 0.1 mg/ml to about 1,000 mg/ml. Suitable concentrations also include about 1 mg/ml to about 1,000 mg/ml, 1 mg/ml to about 500 mg/ml, 1 mg/ml to about 300 mg/ml, 1 mg/ml to about 200 mg/ml, about 1 mg/ml to about 100 mg/ml, about 10 mg/ml to 100 mg/ml, about 10 mg/ml to about 200 mg/ml, about 100 mg/ml to about 500 mg/ml, about 50 mg/ml to about 150 mg/ml, about 20 mg/ml to about 70 mg/ml, about 4 mg/ml to about 20 mg/ml, or about 40 mg/ml to about 66 mg/ml of solubilized ECM. The mammalian acoustic ECM hydrogel can include solubilized ECM at a concentration of about 10 mg/ml to about 500 mg/ml in the liquid, such as the buffer. The mammalian acoustic ECM hydrogel can include 10, 25, 30, 35, 40, 45, 50, 55, 60, 65, 70, 75, 80, 85, 90, 95, 100, 105, 110, 115, 120, 125, 130, 135, 140, 150, 155, 160, 165, 170, 175, 180, 185, 190, 195, and 200 mg/ml solubilized ECM. Exemplary concentrations include about 20 mg/ml, 40 mg/ml, 66 mg/ml, 70 mg/ml, and 150 mg/ml solubilized ECM. In one non-limiting example, the mammalian acoustic ECM hydrogel includes solubilized ECM at a concentration of about 20 mg/ml to about 70 mg/ml. In one non-limiting example, the mammalian acoustic ECM hydrogel includes solubilized ECM a concentration of about 40 mg/ml or about 66 mg/ml. In one non-limiting example, the mammalian acoustic ECM hydrogel includes solubilized ECM at a concentration of about 10 mg/ml to about 100 mg/ml. In one non-limiting example, the mammalian acoustic ECM hydrogel includes solubilized ECM at a concentration of about 50 mg/ml to about 150 mg/ml. In one non-limiting example, the mammalian acoustic ECM hydrogel includes solubilized ECM at a concentration of about 10 mg/ml to about 200 mg/ml. In one non-limiting example, the mammalian acoustic ECM hydrogel includes solubilized ECM at a concentration of about 10 mg/ml to about 500 mg/ml.

Exemplary concentrations include about 20 mg/ml, 40 mg/ml, 66 mg/ml, 70 mg/ml, and 150 mg/ml of solubilized ECM. In one non-limiting example, the mammalian acoustic ECM hydrogel includes about 20 mg/ml to about 70 mg/ml solubilized ECM. In one non-limiting example, the mammalian acoustic ECM hydrogel includes about 40 mg/ml or about 66 mg/ml of solubilized ECM.

In some aspects, the mammalian acoustic ECM hydrogel includes solubilized ECM at a concentration of about 25 mg/ml to about 600 mg/ml. In further aspects, the mammalian acoustic ECM hydrogel includes solubilized ECM at a concentration of about 20 mg/ml to about 600 mg/ml, about 25 to about 500 mg/ml, about 25 to about 400 mg/ml, about 25 mg/ml to about 300 mg/ml, about 25 mg/ml to about 200 mg/ml, and about 25 mg/ml to about 150 mg/ml. In more aspects, the mammalian acoustic ECM hydrogel includes solubilized ECM at a concentration of about 50 mg/ml to 600 mg/ml. The mammalian acoustic ECM hydrogel also can include solubilized ECM at a concentration of about 50 mg/ml to about 300 mg/ml, about 50 mg/ml to about 200 mg/ml, about 50 mg/ml to about 150 mg/ml, about 50-100 mg/ml, or about 100-150 mg/ml. In some non-limiting examples, the mammalian acoustic ECM hydrogel includes solubilized ECM at a concentration of about 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, and 195-200 mg/ml

In some aspects, a composition is produced that includes the mammalian acoustic ECM hydrogel and trehalose. In more aspects, a composition is used includes about 0.1 mg/ml to about 700 mg/ml of trehalose. In some aspects, the composition includes about 1 mg/ml trehalose to about 700 mg/ml trehalose. In further aspects, the composition includes 50 mg/ml to about 500 mg/ml trehalose. In other aspects, the composition includes about 10 mg/ml trehalose to about 600 mg/ml, about 10 mg/ml to about 500 mg/ml, about 10 mg/ml to about 400 mg/ml, about 10 mg/ml to about 300 mg/ml, about 10 mg/ml to about 200 mg/ml, or about 10 mg/ml to about 100 mg/ml trehalose. In further aspects, the composition can include about 0.1 to about 100 mg/ml trehalose, about 0.1 to about 10 mg/ml trehalose, or about 0.1 to about 1 mg/nil trehalose. In more aspects, the composition can include about 50 mg/ml to about 400 mg/ml trehalose, about 50 mg/ml to about 300 mg/ml trehalose, about 50 mg/ml to about 200 mg/ml trehalose, or about 50 ml/ml to about 100 mg/ml trehalose. In some aspects, the composition includes about 20 mg/ml to about 70 mg/ml trehalose. In some aspects, the composition includes about 10 mg/ml to about 100 mg/ml trehalose. In some aspects, the composition includes 15-30 mg/ml trehalose. In some aspects, the composition includes 60-70 mg/ml trehalose. In some aspects, the composition includes 20 mg/ml trehalose. In some aspects, the composition includes 66 mg/ml trehalose. In other aspects, the composition can include about 1, 2, 3, 4, 5, 6, 7, 8, 9, 10, 20, 30, 40, 50, 60, 66, 70, 80, 90, 100, 200, 300, 400, 500, or 600 mg/ml of trehalose. In other aspects, the composition includes about 100 mg/ml to about 700 mg/ml trehalose, such as about 100, 150, 20, 250, 300, 350, 400, 450, 500, 550, or 600 mg/ml trehalose. In more aspects, the composition can include about 0.1, 0.2, 0.3, 0.4, 0.5, 0.6, 0.7, 0.8, 0.9 or 1 mg/ml trehalose.

In more aspects, the composition includes the mammalian acoustic ECM hydrogel comprising solubilized ECM, additional comminuted mammalian ECM, and optionally trehalose. Comminuted ECM is not treated with ultrasound, and is not solubilized into the hydrogel. The comminuted ECM is a distinct additive to composition that also includes the mammalian ECM hydrogel. The composition can include about 1 to about 30% comminuted ECM, weight per volume (w/v), that is not solubilized in the acoustic ECM hydrogel. Without being bound by theory, comminuted ECM generally has intact collagen particles, whereas an acoustic ECM hydrogel has collagen that has been disrupted by ultrasound resulting in an increase in soluble collagen content (Hussey et al., Ultrasonic cavitation to prepare ECM hydrogels Acta Biomater. 2020 May; 108:77-86, incorporated herein by reference in its entirety). As such, an acoustic ECM hydrogel composition containing additional comminuted mammalian ECM includes both intact collagen and disrupted collagen.

The composition can include about 5% to about 30% w/v, about 10% to about 30%, about 15% to about 30%, about 20% to about 30%, about 25% to about 30%, about 1% to about 20%, about 5% to about 20%, about 10% to about 20%, about 15% to about 20%, about 10% to about 20%, or about 15% to about 20% comminuted ECM (w/v). The composition can include about 1, 2, 3, 4, 5, 6, 7, 8, 9, 10, 15, 20, 25, or 30% comminuted ECM (w/v). The composition can include no more than 1, 2, 3, 4, 5, 6, 7, 8, 9, 10, 15, 20, 25, or 30% comminuted ECM (w/v). The composition can include at least about 1, 2, 3, 4, 5, 6, 7, 8, 9, 10, 15, 20, 25, or 30% comminuted ECM (w/v).

Comminuted ECM can be from the same species as the mammalian acoustic ECM hydrogel. In one specific non-limiting example, both the mammalian acoustic ECM hydrogel and the comminuted ECM are porcine. In other non-limiting examples, both the mammalian acoustic ECM hydrogel and the comminuted ECM are human.

The comminuted ECM can be from the same or different tissue as the mammalian acoustic ECM hydrogel. In one aspect, the mammalian acoustic ECM hydrogel and the comminuted ECM are from the same tissue. In one aspect, the mammalian acoustic ECM hydrogel and the comminuted ECM are dermal ECM. In one aspect, the mammalian acoustic ECM hydrogel and the comminuted ECM are porcine dermal ECM.

The composition can be sterilized prior to application to a subject. The composition can be sterilized using any methods known to those of skill in the art, including filtration and radiation. In some aspects, the composition is sterilized with ionizing radiation, such as e-beam or gamma radiation. The composition can be sterilized using gamma radiation, for example, the composition is sterilized using 10 to 50 kGy irradiation, such as 15 to 45 kGy irradiation, 20 to 40 kGy irradiation, or 10 to 30 kGy of irradiation. In some non-limiting examples, the composition is sterilized using 10, 15, 20, 25, 30, 35, 40, 45 or 50 kGy irradiation. Generally, the composition is sterilized for a sufficient time to achieve an absence of detectable viable pathogens, such as, but not limited to, viruses and bacteria.

### Preparation of Enzymatic ECM Hydrogels

Methods of preparing ECM hydrogels, are disclosed herein above, and also, for example, in U.S. Pat. No. 8,361,503, the contents of which is incorporated by reference herein for all purposes. Any type of extracellular matrix tissue can be used to produce a hydrogel which can be used in the methods as disclosed herein (see U.S. Pat. Nos. 4,902,508; 4,956,178; 5,281,422; 5,352,463; 5,372,821; 5,554,389; 5,573,784; 5,645,860; 5,771,969; 5,753,267; 5,762,966; 5,866,414; 6,099,567; 6,485,723; 6,576,265; 6,579,538; 6,696,270; 6,783,776; 6,793,939; 6,849,273; 6,852,339; 6,861,074; 6,887,495; 6,890,562; 6,890,563; 6,890,564; and 6,893,666 related to ECM). In certain aspects, the ECM is isolated from a vertebrate animal, for example and without limitation, from a warm blooded mammalian vertebrate animal including, but not limited to, humans, monkeys, horses, pigs, cows and sheep. In specific non-limiting examples, the ECM is porcine or human.

The ECM can be derived from any organ or tissue, including without limitation, urinary bladder, intestine, liver, esophagus and dermis. For example, the ECM can be derived from urinary bladder, small intestine, heart, dermis, liver, kidney, uterus, brain, blood vessel, lung, bone, muscle, pancreas, placenta, stomach, spleen, colon, adipose tissue, and/or esophagus. The ECM can be obtained from a cell culture. In one aspect, the ECM is isolated from a urinary bladder. In another aspect, the ECM is from an esophagus. The ECM may or may not include the basement membrane portion of the ECM. In certain aspects, the ECM includes at least a portion of the basement membrane.

In some aspects, as U.S. Pat. No. 8,361,503 (incorporated herein by reference), a urinary bladder ECM, such as porcine bladder ECM is prepared by abrading bladder tissue to remove the outer layers including both the tunica serosa and the tunica muscularis using a longitudinal wiping motion with a scalpel handle and moistened gauze. Following eversion of the tissue segment, the luminal portion of the tunica mucosa is delaminated from the underlying tissue using the same wiping motion. In some aspects, perforation of the submucosa is prevented. After these tissues are removed, the resulting ECM consists mainly of the tunica submucosa. The production of hydrogels from decellularized dermal ECM is disclosed in Wolf et al., Biomaterials 33: 7028-7038, 2012, incorporated herein by reference. The production of ECM from esophageal tissue is disclosed, for example, in Badylak et al. J Pediatr Surg. 35(7):1097-103, 2000 and Badylak et al., J Surg Res. 2005 September; 128(1):87-97, 2005, both incorporated herein by reference. U.S. Pat. No. 6,893,666, incorporated herein by reference, discloses production of ECM from urinary bladder, skin, esophagus and small intestine.

Commercially available ECM preparations can also be used in the methods, devices and compositions described herein. In one aspect, the ECM is derived from small intestinal submucosa or SIS. Commercially available preparations include, but are not limited to, SURGISIS™, SURGISIS-ES™, STRATASIS™, and STRATASIS-ES™ (Cook Urological Inc.; Indianapolis, Ind.) and GRAFTPATCH™ (Organogenesis Inc.; Canton Mass.). In another aspect, the ECM is derived from dermis. Commercially available preparations include, but are not limited to PELVICOL™ (sold as PERMACOL™ in Europe; Bard, Covington, Ga.), REPLIFORM™ (Microvasive; Boston, Mass.) and ALLODERM™ (LifeCell; Branchburg, N.J.). In another aspect, the ECM is derived from urinary bladder. Commercially available preparations include, but are not limited to UBM (Acell Corporation; Jessup, Md.).

Tissue for preparation of ECM can be harvested in a large variety of ways and once harvested, a variety of portions of the harvested tissue may be used. ECM has also been prepared from the esophagus and small intestine, and hydrogels have been prepared from this ECM, see, for example, Keane et al., Tissue Eng. Part A, 21(17-18): 2293-2300, 2015, incorporated herein by reference. Esophageal ECM can be prepared by mechanically separating the mucosa and submucosa from the muscularis externa and digesting the mucosal layers in a buffer including trypsin, followed by exposure to sucrose, TRITON-X100®, deoxycholic acid, peracetic acid and DNAse. Small intestine submucosa (SIS) can be prepared by mechanically removing the superficial layers of the tunica mucosa, tunica serosa, and tunica muscularis externa from the intact small intestine, leaving the submucosa, muscularis mucosa, and basilar stratum compactum intact. The SIS is then treated with peracetic acid. Exemplary protocols are provided in Keane et al. Dermal hydrogels can be produced, for example, as disclosed in Wolf et al, J Biomed Mater Res A. 2013. 35(25):6838-49. PMID: 23873846. PMCID: 3808505, incorporated herein by reference.

In one aspect, the ECM is isolated from harvested porcine urinary bladder to prepare urinary bladder matrix (UBM). Excess connective tissue and residual urine are removed from the urinary bladder. The tunica serosa, tunica muscularis externa, tunica submucosa and most of the muscularis mucosa can be removed by mechanical abrasion or by a combination of enzymatic treatment, hydration, and abrasion. Mechanical removal of these tissues can be accomplished by abrasion using a longitudinal wiping motion to remove the outer layers (particularly the abluminal smooth muscle layers) and even the luminal portions of the tunica mucosa (epithelial layers). Mechanical removal of these tissues is accomplished by removal of mesenteric tissues with, for example, Adson-Brown forceps and Metzenbaum scissors and wiping away the tunica muscularis and tunica submucosa using a longitudinal wiping motion with a scalpel handle or other rigid object wrapped in moistened gauze. The epithelial cells of the tunica mucosa can also be dissociated by soaking the tissue in a de-epithelializing solution, for example and without limitation, hypertonic saline. The resulting UBM comprises basement membrane of the tunica mucosa and the adjacent tunica propria, which is further treated with peracetic acid, lyophilized and powdered, see U.S. Pat. No. 8,361,503, incorporated herein by reference.

Dermis sections can used for the preparation of the enzymatic ECM hydrogels, see PCT Application No. 2015/15164728, incorporated herein by reference. In a specific non-limiting example, the dermis can be decellularized with 0.25% Trypsin/1% Triton X-100 (i.e. no SDS) on a vortex shaker at 300 RPM at room temperature in the following solutions: 0.25% trypsin for 6 hours, 1×; deionized water, 15 minutes, 3×; 70% ethanol, 10 to 12 hours, 1×; 3% H₂O₂, 15 minutes, 1×, deionized water, 15 minutes, 2×; 1% Triton X-100 in 0.26% EDTA/0.69% Tris, 6 hours, 1× and then overnight, 1×; deionized water, 15 minutes, 3×; 0.1% peracetic acid/4% ethanol, 2 hours, 1×; PBS, 15 minutes, 2×; and finally deionized water, 15 minutes, 2×. Dermis sheets are then lyophilized and subsequently reduced to particulate form using a Waring blender and a Wiley Mill with a #20 mesh screen.

In some aspects, the epithelial cells can be delaminated first, by first soaking the tissue in a de-epithelializing solution such as hypertonic saline, for example and without limitation, 1.0 N saline, for periods of time ranging from 10 minutes to 4 hours. Exposure to hypertonic saline solution effectively removes the epithelial cells from the underlying basement membrane. The tissue remaining after the initial delamination procedure includes epithelial basement membrane and the tissue layers abluminal to the epithelial basement membrane. This tissue is next subjected to further treatment to remove the majority of abluminal tissues but not the epithelial basement membrane. The outer serosal, adventitial, smooth muscle tissues, tunica submucosa and most of the muscularis mucosa are removed from the remaining deepithelialized tissue by mechanical abrasion or by a combination of enzymatic treatment, hydration, and abrasion.

ECM can be sterilized by any number of standard techniques, including, but not limited to, exposure to peracetic acid, low dose gamma radiation, gas plasma sterilization, ethylene oxide treatment or electron beam treatment. More typically, sterilization of ECM is obtained by soaking in 0.1% (v/v) peracetic acid, 4% (v/v) ethanol, and 95.9% (v/v) sterile water for two hours. The peracetic acid residue is removed by washing twice for 15 minutes with PBS (pH=7.4) and twice for 15 minutes with sterile water. ECM material can be sterilized by propylene oxide or ethylene oxide treatment, gamma irradiation treatment (0.05 to 4 mRad), gas plasma sterilization, peracetic acid sterilization, or electron beam treatment. The ECM can also be sterilized by treatment with glutaraldehyde, which causes cross-linking of the protein material, but this treatment substantially alters the material such that it is slowly resorbed or not resorbed at all and incites a different type of host remodeling which more closely resembles scar tissue formation or encapsulation rather than constructive remodeling. Cross-linking of the protein material can also be induced with carbodiimide or dehydrothermal or photooxidation methods. As disclosed in U.S. Pat. No. 8,361,503, ECM is disinfected by immersion in 0.1% (v/v) peracetic acid (a), 4% (v/v) ethanol, and 96% (v/v) sterile water for 2 h. The ECM material is then washed twice for 15 min with PBS (pH=7.4) and twice for 15 min with deionized water.

Following isolation of the tissue of interest, decellularization is performed by various methods, for example and without limitation, exposure to hypertonic saline, peracetic acid, TRITON-X® or other detergents. Sterilization and decellularization can be simultaneous. For example, and without limitation, sterilization with peracetic acid, described above, also can serve to decellularize the ECM. Decellularized ECM can then be dried, either lyophilized (freeze-dried) or air dried. Dried ECM can be comminuted by methods including, but not limited to, tearing, milling, cutting, grinding, and shearing. The comminuted ECM can also be further processed into a powdered form by methods, for example and without limitation, such as grinding or milling in a frozen or freeze-dried state. In order to prepare solubilized ECM tissue, comminuted ECM is digested with an acid protease in an acidic solution to form a digest solution. The acid protease may be trypsin and/or pepsin, for example, or a combination thereof.

In one aspect, the decellularized ECM material is partially digested by the acid protease. In one example, the decellularized ECM material is digested less completely than a digestion of 1 mg/mL lyophilized, powdered ECM material with 1 mg/mL pepsin in 0.01 M HCl for 48 hours. In another example, the decellularized ECM material is digested less completely than a digestion of 10 mg/mL lyophilized, powdered ECM material with 1 mg/mL pepsin in 0.01 M HCl for 48 hours. In one further aspect, hyaluronic acid in the ECM material is digested less than 50%, 40%, 30%, 25%, 20% or 10% as compared to undigested ECM material, see PCT Application No. WO 2015/164728, incorporated herein by reference.

The digest solution of ECM typically is kept at a constant stir for a certain amount of time at room temperature. The ECM digest can be used immediately or be stored at −20 °C or frozen at, for example and without limitation, −20 °C or −80 °C Thus, the ECM digest can be kept in a solubilized form. Methods for keeping a hydrogel in a solubilized form are disclosed, for example, in PCT Application No. PCT/US16/52261, filed Sep. 10, 2016, incorporated herein by reference.

Once the ECM is solubilized (typically substantially completely) the pH of the solution is raised to between 7.2 and 7.8, and according to one aspect, to pH 7.4. The pH can be raised to about 7.2, 7.3, 7.4, 7.5, 7.6, 7.7 or 7.8. Bases, such as bases containing hydroxyl ions, including NaOH, can be used to raise the pH of the solution. Likewise buffers, such as an isotonic buffer, including, without limitation, Phosphate Buffered Saline (PBS), can be used to bring the solution to a target pH, or to aid in maintaining the pH and ionic strength of the gel to target levels, such as physiological pH and ionic conditions. This forms a “pre-gel” solution which is a solubilized ECM hydrogel. The neutralized digest solution (pre-gel, solubilized ECM hydrogel) can be gelled at lower critical solution temperature, see PCT Publication No. 2015/164728, incorporated herein by reference.

The ECM hydrogel forms a gel (sol to gel transition) upon an increase in temperature. The lower critical solution temperature (LCST) in a reverse gel is a temperature below which a reverse-gelling polymer is soluble in its solvent (e.g. water or an aqueous solvent). As the temperature rises above the LCST in a reverse gel, a hydrogel is formed. The general concept of reverse gelation of polymers and its relation to LCST are broadly known in the chemical arts. The ECM gels described herein are prepared, for example from decellularized, intact ECM as described below, by digestion of the ECM material with an acid protease, neutralization of the material to form a pre-gel, raising the temperature of the pre-gel above the LCST of the pre-gel to cause the pre-gel to gel, such as to form a hydrogel. The transition temperature for acid-protease-digested from solution to gel is typically within the range of from 10 °C to 40 °C and any increments or ranges there between, for example from 20 °C to 35 °C For example, the pre-gel can be warmed to 37 °C to form a hydrogel. For example, the pre-gel.

Thus, the ECM typically can be derived from mammalian tissue, such as, without limitation from one of urinary bladder, esophagus, or small intestine. In one specific non-limiting example, the ECM is derived from urinal bladder. According to one aspect, the decellularized ECM material prepared from the tissue is not dialyzed prior to the partial or complete digestion with the acid protease and/or is not dialyzed after digesting with an acid protease and before gelling of the neutralized, digested ECM material.

In one non-limiting aspect, the ECM is lyophilized and comminuted. The ECM is then solubilized with an acid protease in an acidic solution to produce digested ECM, such as urinary bladder ECM. The acid protease may be, without limitation, pepsin or trypsin, or a combination thereof. The ECM can then be solubilized at an acid pH suitable or optimal for the protease, such as greater than about pH 2, or between pH and 4, for example in a 0.01M HCl solution. The ECM is typically is solubilized for about 12 to about 48 hours, depending upon the tissue type (e.g., see examples below), with mixing (stirring, agitation, admixing, blending, rotating, tilting, etc.). ECM hydrogel is prepared by (i) comminuting an extracellular matrix, (ii) solubilizing intact, non-dialyzed or non-cross-linked extracellular matrix by digestion with an acid protease in an acidic solution to produce a digest solution, (iii) raising the pH of the digest solution to a pH between 7.2 and 7.8 to produce a neutralized digest solution (pre-gel solution), and (iv) gelling the solution.

Accordingly, disclosed is a composition of enzymatically digested ECM in an acid solution with an acid protease and containing exogenous MBV. When, neutralized, e.g., to pH 7.0-7.8, and when warmed to about 37 °C, the composition forms a gel and the proteases are inactivated. In one aspect, the exogenous MBV are not derived from bone or cardiac tissue. In a further aspect, the concentration of exogenous MBV in the composition is greater than 5 mg/mL.

Also disclosed is a composition of an enzymatically digested ECM in a neutral solution, e.g., pH 7.0-7.8, where the solution contains inactivated acid proteases, e.g., inactivated pepsin and/or trypsin, or another inactivated acid protease that in its active form is suitable for digesting ECM; the composition also contains exogenous MBV. The solution when warmed to about 37 °C forms a gel. In one aspect, the exogenous MBV are not derived from bone or cardiac tissue. In a further aspect, the concentration of exogenous MBV in the composition is greater than 5 mg/mL. Acid proteases can be inactivated or deactivated due to, e.g., pH changes.

In a further aspect, the ECM hydrogel can be centrifuged, and a soluble fraction is collected. Exemplary methods for fractionation of an ECM hydrogel are disclosed, for example, in PCT Publication No. WO 2015/164728, incorporated herein by reference. The methods disclosed in this PCT publication include partially or completely digesting with an acid protease, such as pepsin, decellularized ECM material prepared from a tissue; neutralizing the digested ECM material to a pH of 7.0-8.0, 7.2-7.8 or 7.4; gelling the neutralized, digested ECM material at a temperature above its Lower Critical Solution Temperature; centrifuging the gelled ECM material to produce a pellet and a supernatant; and separating the supernatant and the pellet thereby separating a structural and a soluble fraction of the ECM material.

The ECM hydrogel, when exposed to temperatures above the Lower Critical Solution Temperature, such as a temperature of about 37 °C, forms the gel. The ECM hydrogel in the “pre-gel” form (the solubilized ECM hydrogel), can be frozen and stored at, for example and without limitation, −20 °C or −80 °C The ECM hydrogel in the “pre-gel” form can be stored at room temperature, such about 25 °C In some non-limiting examples, the ECM hydrogel is in the pre-gel form at below 37 °C, such as at 25, 24, 23, 22, 21, 20, 19, 18, 17, 16, 15, 14, 13, 12, 11, 10, 9, 8, 7, 6, 5, 4 °C The ECM hydrogel can be frozen for storage, and thus, can be stored at below 0 °C As used herein, the term “pre-gel form” or “pre-gel” refers to the ECM hydrogel wherein the pH is increased, but has not gelled. For example, and without limitation, an ECM hydrogel in the pre-gel form has a pH between 7.2 and 7.8. In some aspects, the solubilized ECM hydrogel is used in the methods disclosed herein. Methods for keeping a hydrogel in a solubilized form are disclosed, for example, in PCT Application No. PCT/US16/52261, filed Sep. 10, 2016, incorporated herein by reference. In some aspects, the ECM composition prepared by any method described herein is absorbed into, adsorbed onto, or otherwise dispersed onto or into a biocompatible substrate. Non-limiting examples of a biocompatible substrate include: a mesh, a non-woven, decellularized tissue, a polymer composition, a polymeric structure, a cell growth scaffold, an implant, an orthopedic implant, and intraocular lens, sutures, intravascular implants, stents, and transplants. The compositions described herein can be applied to or incorporated into, by any suitable method, a non-woven material, such as a bandage, a suture, an implant, such as a ceramic, metal, or polymeric implant, for example a prosthesis, artificial or otherwise-modified vessel, a valve, an intraocular lens, or a tissue implant. As used herein, the term “coat”, and related cognates such as “coated” and “coating,” refers to a process comprising of covering, in part or in whole, an inorganic structure with a composition described herein. For example and without limitation, coating of an inorganic structure with solubilized fraction can include methods such as pouring, embedding, layering, dipping, spraying. Ultrasonication may be used to aid in coating of an inorganic structure.

Compositions of use include ECM hydrogels that are “enzymatic” ECM hydrogels containing exogenous MBV. Exogenous MBV are added to enrich the bioactive properties of the ECM hydrogels and improve their therapeutic efficacy in reducing inflammation and enhancing tissue growth and repair when administered to or implanted in a subject. Enzymatic ECM hydrogels are made from solubilized ECM. In order to prepare solubilized ECM tissue, comminuted ECM is digested with an acid protease in an acidic solution to form a digest solution. As used herein, the term “acid protease” refers to an enzyme that cleaves peptide bonds, wherein the enzyme has increased activity of cleaving peptide bonds in an acidic pH. For example and without limitation, acid proteases can include pepsin and trypsin. In one aspect, the ECM is lyophilized prior to comminution.

The digest solution of ECM typically is kept at a constant stir for a certain amount of time at room temperature. The ECM digest can be used immediately or be stored at −20 °C or frozen at, for example and without limitation, −20 °C or −80 °C To form a “pre-gel” solution, the pH of the digest solution is raised to a pH between 7.2 and 7.8. The pH can be raised by adding one or more of a base or an isotonic buffered solution, for example and without limitation, NaOH or PBS at pH 7.4. The method typically does not include a dialysis step prior to gelation, yielding a more-complete ECM-like matrix that typically gels at 37 °C more slowly than comparable collagen or dialyzed ECM preparations. The gel is therefore more amenable to injection into a patient, and also retains more of the qualities of native ECM due to retention of many native soluble factors, such as, without limitation, cytokines.

As used herein, the term “isotonic buffered solution” refers to a solution that is buffered to a pH between 7.2 and 7.8 and that has a balanced concentration of salts to promote an isotonic environment. As used herein, the term “base” refers to any compound or a solution of a compound with a pH greater than 7. For example and without limitation, the base is an alkaline hydroxide or an aqueous solution of an alkaline hydroxide. In certain aspects, the base is NaOH or NaOH in PBS.

This “pre-gel” solution can, at that point be incubated at a suitably warm temperature, for example and without limitation, at about 37 °C to gel. The pre-gel can be frozen and stored at, for example and without limitation, −20 °C or −80 °C As used herein, the term “pre-gel solution” or “pre-gel” refers to a digest solution wherein the pH is increased. For example and without limitation, a pre-gel has a pH between 7.2 and 7.8. The ECM hydrogel compositions may include inactivated acid protease. The ECM hydrogel compositions may have a pH between 7.2 and 7.8. The “pre-gel” may contain exogenous MBV. In one aspect, the exogenous MBV are not derived from cardiac or bone ECM.

The ECM hydrogel, the digest solution or pregel may contain solubilized ECM at a concentration of between 1 mg/mL and 500 mg/mL. In some aspects, the amount of solubilized ECM in the ECM hydrogel, the digest solution or pregel is between 1 mg/mL and 400 mg/mL, e.g., 1 mg/mL to 350 mg/mL, or 1 mg/mL to 300 mg/mL, or 1 mg/mL to 250 mg/mL or 1 mg/mL to 200 mg/mL or 1 mg/mL to 150 mg/mL or 1 mg/mL to 100 mg/mL or 1 mg/mL to 50 mg/mL, or 5 mg/mL to 250 mg/mL or 20 mg/mL to 200 mg/mL or 5 mg/mL to 200 mg/mL or 5 mg/n to 100 mg/mL. In more aspects, the amount of solubilized ECM in the ECM hydrogel, the digest solution or pregel is between about 5 mg/ml to about 50 mg/ml, such as about 10 mg/ml to about 50 mg/ml, about 20 mg/ml to about 50 mg/ml, about such as about 30 mg/nil to about 50 mg/ml, about 40 mg/ml to about 50 mg/ml, about 5 mg/ml to about 40 mg/ml, about 5 mg/ml to about 30 mg/ml, about 5 mg/ml to about 20 mg/mg, or about 5 mg/ml to about 10 mg/nil. For example, the ECM hydrogel, the digest solution or pregel may contain solubilized ECM at a concentration of about 5, 10, 15, 20, 25, 30, 35, 40, 45, 50, 60, 65, 70, 75, 80, 85, 90, 95, 100, 150, 200, 250, 300, 350, 400, 450, or 500 mg/nil. In one non-limiting example, the amount of solubilized ECM in the ECM hydrogel, the digest solution or pregel is between 10 mg/mL and 30 mg/mL. In another non-limiting example, the amount of solubilized ECM in ECM hydrogel, the digest solution or pregel is between 1 mg/mL and 20 mg/mL. In yet another non-limiting example, the amount of solubilized ECM in the ECM hydrogel, the digest solution or pregel is between 4 mg/mL and 20 mg/mL. In another non-limiting example, the amount of solubilized ECM in the ECM hydrogel, the digest solution or pregel is between 1 mg/mL and 50 mg/mL. In some aspects, the amount of solubilized ECM in the ECM hydrogel, the digest solution or pregel is about 5 to about 100 mg/mL, e.g., 50 to 100 mg/mL, 25 to 75 mg/mL, 60 to 80 mg/mL, 40 to 60 mg/mL, 50 to 80 mg/mL, or about 30 to about 60 mg/mL.

### Preparation of ECM Hydrogels Containing Exogenous MBV

Exogenous MBV can be added to ECM hydrogels (or pregel), e.g., enzymatic hydrogels or acoustic hydrogels as disclosed herein. The exogenous MBV may be present in the ECM hydrogel at a concentration of less than 1 mg/mL. For example, the MBV may be present in the ECM hydrogel at a concentration of at least about 1×10⁵ to about 1×10²⁰ particles/mL. In some aspects, the exogenous MBV are present in the ECM hydrogels (or pregel) at a concentration of about 1×10⁵ to about 1×10²⁰ particles/mL, such as about 1×10⁵ to about 1×10¹⁸ particles/mL such as about 1×10⁵ to about 1×10¹⁶ particles/mL, such as about 1×10⁵ to about 1×10¹⁴ particles/mL, or such as about 1×10⁵ to about 1×10¹² particles/mL. In some aspects, the exogenous MBV are present in the ECM hydrogels (or pregel) at a concentration of about 1×10⁶ to about 1×10²¹ particles/mL, such as about 1×10⁶ to about 1×10¹⁸ particles/mL, such as about 1×10⁶ to about 1×10¹⁶ particles/mL, such as about 1×10⁶ to about 1×10¹⁴ particles/mL, such as about 1×10⁶ to about 1×10¹² particles/mL, such as about 1×10⁷ to about 1×10¹² particles/mL, such as about 1×10⁷ to about 1×10¹¹ particles/mL, such as about 1×10⁸ to about 1×10¹² particles/mL, such as about 1×10⁸ to about 1×10¹¹ particles/mL, such as about 1×10⁹ to about 1×10¹² particles/mL, such as about 1×10⁹ to about 1×10¹¹ particles/mL, such as about 1×10¹⁰ to about 1×10¹² particles/mL, such as about 1×10¹⁰ to about 1×10¹¹ particles/mL, such as about 1×10¹¹ to about 1×10¹² particles/mL. In one non-limiting example, the exogenous MBV are present at a concentration of about 1×10⁸ to 1×10¹¹ particles/mL. In one non-limiting example, the exogenous MBV are present at a concentration of about 1×10⁵ to 1×10¹² particles/mL. In one non-limiting example, the exogenous MBV are present in an ECM hydrogel disclosed herein (or pregel) at a concentration of about 1×10⁶ to 1×10¹² particles/mL. In some aspects, the exogenous MBV are present in the ECM hydrogel (or pregel) at a concentration of about 1×10⁶, 1×10⁷, 1×10⁸, 1×10⁹, 1×10¹⁰, 1×10¹¹, 1×10¹², 1×10¹⁴, 1×10¹⁶, 1×10¹⁸, or about 1×10²⁰ particles/mL. In other aspects, the exogenous MBV are present in the ECM hydrogel (or pregel) at a concentration of about 5×10⁶, 5×10⁷, 5×10⁸, 5×10⁹, 5×10¹⁰, 5×10¹¹, or about 5×10¹² particles/mL. In a specific non-limiting example, the exogenous MBV are present in the ECM hydrogel (or pregel) at a concentration of about 1×10¹¹ particles/mL. In another non-limiting example, the exogenous MBV are present in the ECM hydrogel (or pregel) at a concentration of about 1×10¹² particles/mL. In a further non-limiting example, the exogenous MBV are present in the ECM hydrogel (or pregel) at concentration of about 1×10¹⁰ particles/mL or about 1×10⁹ particles/mL. In some aspects, the exogenous MBV are present in the ECM hydrogel (or pregel) at a concentration of about 1×10⁶ to 1×10¹⁸ particles/mL, e.g. about 1×10⁶ to 1×10¹⁴, about 1×10¹⁰ to 1×10¹¹, 1×10¹² to 1×10¹², 1×10¹⁴ to 1×10¹⁸, or 1×10¹⁰ to 1×10¹⁸. In another non-limiting example, the exogenous MBV are present in the ECM hydrogel (or pregel) at a concentration of less than 1 mg/mL, e.g., ≤0.9 mg/mL, ≤0.8 mg/mL, ≤0.7 mg/mL, ≤0.6 mg/mL, ≤0.5 mg/mL, ≤0.4 mg/mL, ≤0.3 mg/mL, ≤0.2 mg/mL, ≤0.1 mg/mL, ≤90 μg/mL, ≤80 μg/mL, ≤70 μg/mL, ≤60 μg/mL, ≤50 μg/mL, ≤40 μg/mL, ≤30 μg/mL, ≤20 μg/mL, or ≤10 μg/mL, but greater than 0 μg/mL, e.g., greater than 0.1 μg/mL, or greater than 0.5 μg/mL, or greater than 1 μg/mL.

The MBV may be added to the hydrogel, for example, prior to gelation of an enzymatic hydrogel. For example, the MBV may be added to an enzymatic hydrogel prior to raising the temperature to above 25 °C, e.g., to 37 °C For example, the MBV may be added to an acoustic hydrogel at any point after sonication of the ECM to produce the hydrogel.

### Methods of Treatment

Disclosed herein are a variety of methods for treating a subject having a muscle wasting disease. Various muscle wasting diseases are disclosed herein and these methods may be used to treat any of the disclosed disorders. The methods involve using MBV either directly administered to a subject suffering from the muscle wasting disease, or using MBV in combination with cells to provide cells for adoptive transfer to a subject, or to prepare a conditioned media resulting from culture of cells with MBV for administration to the subject. In some examples, a human subject suffering from a muscle wasting disease is selected, and treated, according to the methods disclosed herein.

Muscle wasting diseases include small muscular atrophy, and muscular dystrophy, as well as other conditions that cause muscular atrophy. For example, the methods of treatment disclosed herein may be used to treat spinal muscular atrophy (“SMA”) such as infantile progressive spinal muscular atrophy (SMA Type I), intermediate spinal muscular atrophy (SMA Type II), juvenile spinal muscular atrophy (SMA Type III), or adult spinal muscular atrophy (SMA Type IV). The methods of treatment disclosed herein may also be used to treat muscular atrophy that occurs in cachexia or sarcopenia. The methods of treatment disclosed herein may also be used to treat muscular dystrophies (MD), such as Becker MD, Congenital MD, Duchenne MD, Distal MD, Emery-Dreifuss MD, Facioscapulohumeral MD (FSHD), Limb-Girdle MD, Myotonic MD, Oculopharyngeal MD, Bethlem myopathy, or Ullrich Congenital Muscular Dystrophy. For example, the disclosed methods can be used to treat FSHD. For example, the methods disclosed herein can be used to treat SMA. A subject with SMA can be selected for treatment, using the disclosed methods.

Methods of treatment are also disclosed for promoting muscle regeneration or repair in a subject in need thereof, where the subject is administered an effective amount of a composition comprising exogenous matrix bound nanovesicles (MBV) derived from extracellular matrix, wherein the MBV do not express CD63 and CD81 or are CD63^{lo}CD81^{lo} and wherein the MBV do not contain alkaline phosphatase.

### Administration of MBV

In some aspects, disclosed is a method of treating a subject with a muscle wasting condition, the method comprising administering to the subject a composition comprising an effective amount of exogenous matrix bound nanovesicles (MBV) derived from extracellular matrix, wherein the MBV do not express CD63 and CD81 or are CD63^{lo}CD81^{lo} and wherein the MBV do not contain alkaline phosphatase. In more aspects, disclosed is a method of promoting muscle regeneration or repair in a subject in need thereof, where the subject is administered an effective amount of a composition comprising exogenous matrix bound nanovesicles (MBV) derived from extracellular matrix, wherein the MBV do not express CD63 and CD81 or are CD63^{lo}CD81^{lo} and wherein the MBV do not contain alkaline phosphatase. In all of these aspects, the MBV may be prepared as disclosed herein.

The MBV may be administered in an ECM hydrogel as disclosed herein, for example, an enzymatic hydrogel, or an acoustic hydrogel. The enzymatic hydrogel may be in a pregel state. The exogenous MBV contained in the hydrogel may be present in the hydrogel in quantities as described herein. The ECM hydrogel containing MBV may be administered systemically, e.g., intravenously. For example, the ECM hydrogel containing MBV may be administered locally to a muscle affected by atrophy or dystrophy, as in any of the muscle wasting disorders described herein. The amount of hydrogel administration will depend on the concentration of MBV in the hydrogel and desired outcomes in the patient including disease severity.

MBV may also be suspended in a pharmaceutically acceptable carrier. For example, MBV may be provided in a carrier, such as a balanced salt solution, or physiological saline, that may be isotonic, hypotonic, or hypertonic depending on the needs of the subject. For example, the salt solution maybe a magnesium chloride solution, sodium chloride solution, potassium chloride solution or a calcium chloride solution. The carrier may be lactated Ring's solution, or a dextrose or other sugar solution. The carrier may be buffered as appropriate to maintain physiologic pH in the subject. The carrier may include glycerol. MBV may be provided in the carrier at a concentration of less than 1 mg/mL, e.g., ≤0.9 mg/mL, 0.8 mg/mL, ≤0.7 mg/mL, ≤0.6 mg/mL, ≤0.5 mg/mL, ≤0.4 mg/mL, ≤0.3 mg/mL, ≤0.2 mg/mL, ≤0.1 mg/mL, ≤90 μg/mL, ≤80 μg/mL, ≤70 μg/mL, ≤60 μg/mL, ≤50 μg/mL, ≤40 μg/mL, ≤30 μg/mL, ≤20 μg/mL, or ≤10 μg/mL, but greater than 0 μg/mL, e.g., greater than 0.1 μg/mL, or greater than 0.5 μg/mL, or greater than 1 μg/mL.

The amount of solution administration may be, for example, from 100 microliters to 10 mL depending on the concentration of MBV in the solution and the desired dose.

Administration of MBV, e.g. in a pharmaceutically acceptable carrier, or in a hydrogel, prepared as described herein, can be administered to a subject, e.g., a human subject suffering from a muscle wasting disorder to treat the muscle wasting disorder. The muscle wasting disorder may be, for example, any of those disclosed herein. Administration may be intravenous, or intramuscular, or subcutaneous, for example.

Administration of MBV to the subject may occur daily weekly, every two weeks, monthly (e.g., about every 28 days), every two months (e.g., about every 56 days), or every three months (e.g., about every 84 days). The quantity of MBV administered and the frequency of dosing can be adjusted depending on the desired outcome and the type and severity of the muscle wasting disorder in the subject.

For example, a subject is administered about 1×10¹ to about 1×10²⁰ MBV per kg of body weight per administration. For example, a subject is administered about 1×10⁶ to about 1×10²⁰ MBV/kg of body weight, such as about 1×10⁶ to about 1×10² MBV per kg of body weight per administration. In some examples, a subject is administered about 1×10⁶ to about 1×10¹⁹ MBV, about 1×10⁶ to about 1×10¹⁸ MBV, about 1×10⁶ to about 1×10⁷ MBV, about 1×10⁶ to about 1×10¹⁶ MBV, about 1×10⁶ to about 1×10¹⁵ MBV, about 1×10⁶ to about 1×10¹⁷ MBV, about 1×10⁶ to about 1×10¹⁶ MBV, or about 1×10⁶ to about 1×10¹² MBV per administration. In other examples, a subject is administered about 1×10⁷ to about 1×10¹¹ MBV per kg of body weight per administration. In another example, a subject is administered 1×10⁷ to 1×10⁸ MBV per kg of body weight per administration. In another embodiment, a subject is administered 1×10⁸ to 1×10¹⁰ MBV per kg of body weight per administration. In another embodiment, a subject is administered 1×10⁹ to 1×10¹⁰ MBV per kg of body weight per administration. In another embodiment, a subject is administered 1×10⁶ to 1×10⁸ MBV per kg of body weight per administration. In another embodiment, a subject is administered 1×10⁷ to 1×10⁹ MBV per kg of body weight per administration. In another embodiment, a subject is administered 1×10⁸ to 1×10¹¹ MBV per kg of body weight per administration. In another embodiment, a subject is administered 1×10⁹ to 1×10¹¹ MBV per kg of body weight per administration. In another embodiment, a subject is administered 1×10⁹ to 1×10¹¹ MBV per kg of body weight per administration. In another embodiment, a subject is administered 1×10¹⁰ to 1×10² MBV per kg of body weight per administration. In another embodiment, a subject is administered 1×10⁶ to 1×10¹⁴ MBV per kg of body weight per administration. In another embodiment, a subject is administered 1×10¹² to 1×10¹⁴ MBV per kg of body weight per administration. In one embodiment, administration of MBV according to any of the aforementioned amounts is by systemic administration. For example, in one embodiment, the administration is intravenous. In another embodiment, the administration is by bolus subcutaneous injection, for example, into muscle.

### Adoptive Transfer

In some aspects, disclosed is a method of treating a subject with a muscle wasting condition, the method comprising administering to the subject a composition comprising an effective amount of myeloid progenitor cells or a progeny thereof (a “myeloid-derived cell) that has been treated with exogenous MBV. In some aspects, the myeloid-derived cell is a macrophage. Macrophages are resident phagocytic cells in lymphoid and nonlymphoid tissues with highly diverse roles in the maintenance of an organism's biological integrity ranging from development, homeostasis, to repair, and immune responses to pathogens. Macrophages exert these functions through clearance of cell debris, production of growth factors, highly efficient phagocytosis (notably of tumor cells), and the production of inflammatory cytokines. Macrophages are usually classified into classically activated, pro-inflammatory or M1 macrophages, and alternatively activated, anti-inflammatory, or M2 macrophages. In some aspects, the myeloid cell of use in the disclosed methods in an anti-inflammatory M2 macrophage.

M2 or anti-inflammatory macrophages (also named alternatively-activated macrophages) can be induced by IL-4 or IL-13 secreted by innate and adaptive immune cells, such as mast cells, basophils, and TH-2 lymphocytes. M2a or activated macrophages typically express surface markers comprising CD206, CD36, ILHRa, and CD163; transcription factors comprising STAT6, GATA3, SOCS1 and PPARy; and metabolic enzymes comprising ARG1 and CARKL, and can secrete cytokines including, but not limited to, IL10 and TGFp. M2b or regulatory macrophages are typically induced by stimulation with immune complexes and TLR ligands or by IL-1R agonists. M2b macrophages typically express surface markers comprising CD86 and MHC-II molecules; transcription factors comprising STATS, IRF4 and p50 (NF-kb); and metabolic enzymes comprising ARG1 and CARKL. In some aspect, the myeloid-derived cell is a M2b macrophages. M2c macrophages are activated by glucocorticoids or IL-10 and exhibits a strong anti-inflammatory profile and a phagocytosis activity of apoptotic bodies. M2c macrophages typically express surface markers comprising CD163, TLR1 and TLR8; transcription factors comprising STATS, STAT6, IRF4 and p50 (NF-kb); and metabolic enzymes comprising ARG1 and GS. M2c macrophages typically secrete cytokines comprising IL10 and TGF-b. The expression of surface markers comprising CD14, CD206 and CD163 on M2 macrophages has been described to be associated with phagocytosis competence (Schulz et al. In-Depth Characterization of Monocyte-Derived Macrophages using a Mass Cytometry-Based Phagocytosis Assay. Sci Rep 9, 1925 (2019), doi.Org/10.1038/s41598-018-38127-9). The disclosed methods can utilize M2 macrophages, including M2a, M2b and/or M2c macrophages.

In some aspects myeloid progenitor cells or myeloid-derived cells, such as those isolated directly from a subject (autologous) and/or isolated from a subject and treated with the MBV. In other aspects, the cells are derived from cell lines and treated with the MBV. The cells can also be obtained from a xenogeneic source, such as a mouse, a rat, a non-human primate, or a pig and treated with the MBV. In some aspects, the cells are human cells. In some aspects, the myeloid-derived cells, such as M2 macrophages can be obtained from human induced pluripotent stem cells (IPSCs) (See Hansen et al., Stem cell research vol. 29 (2018): 232-244; Lachmann et al., Stem cell reports vol. 4,2 (2015): 282-96. doi:10.1016/j.stemcr.2015.01.005; Mukherjee et al., A Simple Multistep Protocol for Differentiating Human Induced Pluripotent Stem Cells into Functional Macrophages. In: Rousselet G. (eds) Macrophages. Methods in Molecular Biology, vol 1784. Humana Press, New York, NY.).

With reference to the subject to be treated, the myeloid progenitor cells or myeloid-derived cells can be allogeneic and/or autologous. The cells and compositions are typically isolated from a sample, notably a biological sample, e.g., obtained from or derived from a subject. Typically, the subject is in need for a cell therapy (adoptive cell therapy) and/or is the one who will receive the cell therapy. The subject is preferably a mammal, notably a human.

In autologous adoptive cell transfer, the myeloid progenitor cells or myeloid-derived cells are collected from the subject, treated with MBV, and returned to the subject. In allogeneic adoptive cell transfer, myeloid progenitor cells or myeloid-derived cells are collected from healthy donors, rather than the patient, treated with MBV, and administered to a subject of interest with a muscle wasting disorder to be treated. In some aspects, these allogeneic cells are HLA matched to reduce the likelihood of rejection by the host. The myeloid-derived cell as herein described may thus also comprise modifications such as disruption or removal of HLA class I molecules. For example, Torikai et al., Blood. 2013; 122: 1341-1349 and Ren et al., Clin. Cancer Res. 2017; 23:2255-2266).

In certain aspects, the myeloid progenitor cells or myeloid-derived cells can be obtained from a unit of blood collected, or from bone marrow, or collected from a subject using any number of techniques known to the skilled artisan, such as Ficoll separation for blood cells. In one aspect, cells from the circulating blood of an individual can be obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media. In another aspect, cells can be isolated from peripheral blood by lysing the red blood cells and depleting the lymphocytes and red blood cells, for example, by centrifugation through a PERCOLL™ gradient. Alternatively, cells can be isolated from umbilical cord. In any event, a specific subpopulation of myeloid cells, typically the macrophages can be further isolated by positive or negative selection techniques.

In one aspect, the cells or population of cells comprising myeloid derived cells, such as macrophages, are cultured for expansion. In another aspect, the cells or population of cells comprising myeloid progenitor cells are cultured for differentiation and expansion into myeloid derived cells, such as macrophages. In expanding the cells, the myeloid derived cells, such as macrophages, can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one aspect, the cells expand in the range of about 20 fold to about 50 fold.

Following culturing, the cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. In some aspects, the level of confluence is 70% or greater before passing the cells to another culture apparatus. More preferably, the level of confluence is 90% or greater. A period of time can be any time suitable for the culture of cells in vitro. The culture medium may be replaced during the culture of the cells at any time. In some aspects, the culture medium is replaced about every 2 to 3 days. The cells are treated with an effective amount of MBV, and harvested from the culture apparatus whereupon the cells can be used immediately or stored, such as by freezing, for use at a later time.

The culturing step as described herein (including contact with MBV as described herein) can be very short, for example less than 24 hours such as about 1, 2, 3, 4, 5, 6, 7, 8, 9, 10, 11, 12, 13, 14, 15, 16, 17, 18, 19, 20, 21, 22, 23 or 24 hours. The culturing step as described further herein (contact with MBV as described herein) can be for example 1, 2, 3, 4, 5, 6, 7, 8, 9, 10, 11, 12, 13, 14, or more days.

In one aspect, the myeloid progenitor cells or myeloid derived-cells may be cultured for several hours (about 3 hours) to about 14 days or any hourly integer value in between. The myeloid derived cells can be cultured for 1-10 days, such as 2-9 days, such as 3-8 days, such as 3, 4, 5, 6, 7, 8, or 9 days. In some aspects, the myeloid derived cells are cultured for 7 days. Conditions appropriate for cell culture include an appropriate media (e.g., macrophage complete medium, DMEM/F12, DMEM/F 12-10 (Invitrogen) or DMEM high glucose) that may contain factors necessary for proliferation and viability, including serum (e.g., fetal bovine or human serum), L-glutamine, insulin, M-CSF, GM-CSF, IL-10, IL-12, IL-15, TGF-b, and TNF-a. or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AEVI-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of the cells. Antibiotics, e.g., penicillin and streptomycin, are included only in experimental cultures, not in cultures of cells that are to be infused into a subject. The cells are maintained under conditions necessary to support growth, for example, an appropriate temperature (e.g., 37 °C) and atmosphere (e.g., air plus 5% C02).

Following this period, MBV can be added to the culture media at a concentration of, for example, about 1×10⁶ MBV/ml to about 1×10²⁰ MBV/ml, such as 1×10⁹ MBV/ml to about 1×10¹⁴ MBV/ml, such as about 1×10¹⁰ MBV/ml to about 1×10¹² MBV/ml, or 1×10¹⁰ MBV/ml to about 1×10¹¹ MBV/ml, or 1×10¹¹ MBV/ml to about 1×10¹² MBV/ml, or 1×10¹⁰ MBV/ml to about 1×10¹¹ MBV/ml, or 1×10¹¹ MBV/ml to about 1×10¹⁴ MBV/ml, or 1×10⁹ MBV/ml to about 1×10¹¹ MBV/ml, for example, about 1×10⁹ MBV/ml, about 1×10¹⁰ MBV/ml, about 1×10¹¹ MBV/ml, about 1×10¹² MBV/ml, about 1×10¹³ MBV/ml, or 1×10¹⁴ MBV/ml. In some aspects, the MBV can be added to the culture at about 1×10⁶ to about 1×10¹² MBV/ml. In some examples, the MBV can be added to the culture at a concentration of about 1×10⁶ to about 1×10¹⁹ MBV/ml, about 1×10⁶ to about 1×10¹⁸ MBV/ml, about 1×10⁶ to about 1×10¹⁷ MBV/ml, about 1×10⁶ to about 1×10¹⁶ MBV/ml, about 1×10⁶ to about 1×10¹⁵ MBV/ml, about 1×10⁶ to about 1×10¹⁴ MBV/ml, about 1×10⁶ to about 1×10¹³ MBV/ml, or about 1×10⁶ to about 1×10¹² MBV/ml. In other examples, the MBV can be added to the culture media at a concentration of, for example, about 1×10⁷ to about 1×10¹¹ MBV/ml.

In some aspects, the myeloid derived cells are treated with the MBV for about 2 to about 72 hours, such as for about 12 to about 48 hours, such as for about 24 to about 36 hours. The myeloid progenitor cells or myeloid-derived cells, such as macrophages, for example, M2 macrophages, can be treated with the MBV for about 12, about 24, about 36 or about 48 hours. The myeloid-derived cells, such as macrophages, for example, M2 macrophages, can be treated with the MBV for about 24 hours.

The present method also relates to a method of treatment and notably an adoptive cell therapy, preferably an adoptive myeloid-derived cell therapy, comprising the administration to a subject in need thereof of myeloid progenitor cells or myeloid-derived cells that have been contacted with MBV. The administration can occur, for example, without about 2 to 48 hours following contact of the myeloid progenitor cells or myeloid-derived cells with the MBV, such as about 12 to about 24 hours following contact of the myeloid progenitor cells or myeloid-derived cells with the MBV, such as within about 2, 4, 6, 8, 10, 12, 14, 16, 19, 20, 22 or 24 hours. In other aspects, the myeloid progenitor cells or myeloid-derived cells are frozen following contact with the MBV, as disclosed herein, and then utilized at a later date.

In some aspects, an effective amount of the myceloid progenitor or myeloid derived cells that have been treated with the MBV or compositions including these myeloid-derived cells are administered to the subject, such as a subject having a muscle-wasting condition. In some aspects, the methods thereby treat, e.g., ameliorate one or more symptom of, the muscle wasting condition.

In some aspects, the adoptive cell therapy is carried out by autologous transfer, in which the myeloid progenitor or myeloid-derived cells are isolated and/or otherwise prepared from the subject who is to receive the myeloid-derived cell therapy. Thus, in some aspects, the myeloid progenitor or myeloid derived cells are produced from a subject with the muscle wasting condition. Following isolation and treatment with the MBV, the myeloid progenitor or myeloid derived cells are administered to the same subject. In some aspects, the adoptive cell therapy is carried out by allogeneic transfer, in which the myeloid progenitor or myeloid-derived cells are isolated and/or otherwise prepared from a different subject (a first subject) other than the subject with the muscle wasting condition (a second subject). In such aspects, the myeloid progenitor or myeloid derived cells are treated with the MBV, and then are administered to a different subject, such as, of the same species, with the muscle wasting condition.

In some aspects, the first and second subjects are genetically identical. In some aspects, the first and second subjects are genetically similar. In some aspects, the second subject expresses the same HLA class or supertype as the first subject. In some aspects, HLA matching is less important when the immune cell has been mod ified to reduce expression of endogenous TCR and HLA class I molecules.

Administration of myeloid progenitor or myeloid-derived cells that have been treated with MBV can be combined with one or more additional therapeutic agents or in connection with another therapeutic intervention, either simultaneously or sequentially in any order. In some contexts, the myeloid-derived cells are co-administered with another therapy sufficiently close in time such that the cell populations enhance the effect of one or more additional therapeutic agents, or vice versa. In some aspects, the cell populations are administered prior to the one or more additional therapeutic agents. In some aspects, the cell populations are administered after to the one or more additional therapeutic agents.

Administration of myeloid progenitor or myeloid-derived cells prepared as described herein, can be administered to a subject, e.g., a human subject suffering from a muscle wasting disorder to treat the muscle wasting disorder. The muscle wasting disorder may be, for example, any of those disclosed herein. The myeloid progenitor or myeloid-derived cells can be administered systemically, for example, intravenously.

Myeloid progenitor or myeloid-derived cells that have been treated with MBV can be administered to the subject weekly, every two weeks, monthly (e.g., about every 28 days), every two months (e.g., about every 56 days), every three months (e.g., about every 84 days). The number of cells administered and the frequency of dosing can be adjusted depending on the desired outcome and the type and severity of the muscle wasting disorder in the subject.

### Conditioned Medium

In some aspects, disclosed is a method of treating a subject with a muscle wasting condition, the method comprising administering to the subject a composition comprising an effective amount of conditioned media or a fraction thereof obtained from a culture of macrophages and cultured in the presence of the exogenous MBV.

The isolation of myeloid progenitor or myeloid-derived cells, and treating them with MBV in vitro, is disclosed above. In some aspects, medium conditioned by culturing such cells can be utilized in the disclosed methods.

Additionally, macrophages, such as M2 macrophages, may be cultured in any medium suitable for mammalian cell culture such as but not limited to RPMI-1640 medium optionally with human or fetal bovine serum, or Dulbecco's Modified Eagle Medium/Nutrient Mixture F12 (DMEM-F12) along with MBV to produce a conditioned medium for use in treating muscle wasting conditions.

MBV may be added to condition the culture medium containing the macrophages at a concentration o about 1×10⁶ MBV/ml to about 1×10²⁰ MBV/ml, for example, about 1×10⁹ MBV/ml to about 1×10¹⁴ MBV/ml, such as about 1×10¹⁰ MBV/ml to about 1×10¹² MBV/ml, or 1×10¹⁰ MBV/ml to about 1×10¹¹ MBV/ml, or 1×10¹¹ MBV/ml to about 1×10¹² MBV/ml, or 1×10¹⁰ MBV/ml to about 1×10¹⁴ MBV/ml, or 1×10¹¹ MBV/ml to about 1×10¹⁴ MBV/ml, or 1×10⁹ MBV/ml to about 1×10¹¹ MBV/ml, for example, about 1×10⁹ MBV/ml, about 1×10¹⁰ MBV/ml, about 1×10¹¹ MBV/ml, about 1×10¹² MBV/ml, about 1×10¹³ MBV/ml, or 1×10¹⁴ MBV/ml. In some aspects, the MBV can be added to the culture at about 1×10⁶ to about 1×10¹² MBV/ml. In some examples, the MBV can be added to the culture at a concentration of about 1×10⁶ to about 1×10¹⁹ MBV/ml, about 1×10⁶ to about 1×10¹⁸ MBV/ml, about 1×10⁶ to about 1×10¹¹ MBV/ml, about 1×10⁶ to about 1×10¹⁶ MBV/ml, about 1×10⁶ to about 1×10¹⁵ MBV/ml, about 1×10⁶ to about 1×10¹⁴ MBV/ml, about 1×10⁶ to about 1×10¹³ MBV/ml, or about 1×10⁶ to about 1×10¹² MBV/ml. In other examples, the MBV can be added to the culture media at a concentration of, for example, about 1×10⁷ to about 1×10¹¹ MBV/ml.

In some aspects, the macrophages are cultured in the medium with the MBV for about 2 to about 72 hours, such as for about 12 to about 48 hours, such as for about 24 to about 36 hours. The macrophages, for example, M2 macrophages, can be treated with the MBV for about 12, about 24, about 36 or about 48 hours. The macrophages, for example, M2 macrophages, can be treated with the MBV for about 24 hours.

Once the desired period for preparing the conditioned medium has been reached, e.g., the desired concentration of macrophage secretome molecules are present in the medium has been reached, the culture medium can be separated from the cells to isolate the conditioned media. This can be achieved, for example, through centrifugation and other known techniques. In some examples, when centrifugation is used, the conditioned media will be the supernatant and the macrophages will be in the pellet. The conditioned media is, in some examples, therefore free or substantially free of cells. For example, no more 10%, e.g., no more than 5% of the conditioned media contains cells by e.g., weight, or e.g. volume.

The conditioned medium can be subject to lyophilization for preservation and/or concentration of the bioactive agents that promote tissue repair. A typical lyophilization process comprises three separate and interdependent processes: freezing, primary drying (sublimation), and secondary drying (desorption). Various biocompatible preservatives, cryoprotectants, and stabilizer agents can be used to preserve activity where required. Non-limiting examples of biocompatible agents include, among others, glycerol, dimethyl sulfoxide, and trehalose. The lyophilizate, in some aspects, also includes one or more excipients such as buffers, bulking agents, and tonicity modifiers. The freeze-dried media is reconstituted by addition of a suitable solution or pharmaceutical diluent.

In some aspects, the conditioned medium can be processed by precipitating the bioactive agents (e.g., growth factors, cytokines, and/or Wnt proteins) in the medium. Precipitation may be performed using various procedures, such as salting out with ammonium sulfate or use of hydrophilic polymers, for example polyethylene glycol.

In other aspects, the conditioned medium is subject to filtration using various selective filters. Processing the conditioned medium by filtering is useful in concentrating the factors that promote tissue repair and for removing small molecules and solutes used in the conditioned medium. Filters with selectivity for specified molecular weights include <5000 Daltons, <10,000 Daltons, and <15,000 Daltons. Other filters can be used and the processed media assayed for tissue repair promoting activities as described herein. Exemplary filters and concentrator system include those based on, among others, hollow fiber filters, filter disks, and filter probes.

In still other aspects, the conditioned medium is subject to chromatography to remove salts, impurities, or fractionate various components of the medium. Various chromatographic techniques may be employed, such as molecular sieving, ion exchange, reverse phase, affinity chromatographic techniques. For processing conditioned medium without significant loss of bioactivity, mild chromatographic media is used. Non-limiting examples include, among others, dextran, agarose, polyacrylamide based separation media (e.g., available under various tradenames, such as SEPHADEX® and SEPHAROSE®. In some aspects, impurities are removed from the conditioned medium using the methods disclosed in U.S. Patent Publication No. 2004/0248803.

The conditioned medium can be used directly without the addition of pharmaceutically acceptable carriers, or pharmaceutical compositions can be prepared that include the conditioned medium and various pharmaceutically acceptable carriers. A pharmaceutical composition refers to a form of the conditioned media and at least one pharmaceutically acceptable carrier. The compositions can also contain formulating agents, such as suspending, stabilizing or dispersing agents. Formulations for injection can be presented in unit dosage form, ampules in multidose containers, with or without preservatives. Alternatively, the compositions can be presented in powder form for reconstitution with a suitable vehicle including, by way of example and not limitation, sterile pyrogen free water, saline, buffer, or dextrose solution.

In still other aspects, the conditioned medium comprising the bioactive agents can be introduced or encapsulated into the lumen of liposomes for delivery and for extending the life time of the bioactive agents. Liposomes can be categorized into various types: multilamellar (MLV), stable plurilamellar (SPLV), small unilamellar (SUV) or large unilamellar (LUV) vesicles. Liposomes can be prepared from various lipid compounds, which can be synthetic or naturally occurring, including phosphatidyl ethers and esters, such as phosphotidylserine, phosphatidylcholine, phosphatidyl ethanolamine, phosphatidylinositol, dimyristoylphosphatidylcholine; steroids such as cholesterol; cerebrosides; sphingomyelin; glycerolipids; and other lipids (see, e.g., U.S. Pat. No. 5,833,948).

The conditioned medium can be used alone, or in combination with other compatible bioactive agents useful for treating muscle wasting. In some aspects, the conditioned medium can be used with other compounds or compositions.

The conditioned medium, processed as described herein, can be administered to a subject, e.g., a human subject suffering from a muscle wasting disorder to treat the muscle wasting disorder. The muscle wasting disorder may be, for example, any of those disclosed herein. The conditioned medium can be administered systemically, for example, intravenously or by intramuscular or subcutaneous administration. Intramuscular administration may target a particular muscle of interest suffering from wasting, e.g., a muscle experiencing atrophy or dystrophy.

Conditioned media may be administered to the subject daily, weekly, every two weeks, monthly (e.g., about every 28 days), every two months (e.g., about every 56 days), every three months (e.g., about every 84 days). The amount and frequency of dosing can be adjusted depending on the desired outcome and the type and severity of the muscle wasting disorder in the subject.

### Treatment of SMA

Provided herein are methods for treating a subject (for example, a human subject) with SMA. The methods may be utilized to treat (i.e., prevent, ameliorate, suppress, and/or alleviate) a motor impairment due to SMA in the subject. In aspects, the SMA is type 1 SMA. In other aspects, the SMA is type 2 SMA. In further aspects, the SMA is type 3 or 4 SMA.

Any appropriate subject with or at risk of a motor impairment due to SMA can be treated with the method provided herein. The motor impairment can be in a limb of the upper or lower body, such as an above or below the elbow, or an above or below the knee, or including the entire arm or leg. The subject can have any of SMA types 1-4, such as type 1, type 2, type 3, or type 4. In some implementations, a subject with SMA is selected for treatment. The method can be in initiated at any time post-onset of the motor impairment in the subject, or even in advance of detectable motor impairment in an SMA patient at risk of motor impairment.

SMA may be treated by administering MBV, conditioned media, or myeloid-progenitor cells prepared as disclosed herein to the patient. The administration may be directly to the spinal cord, or musculature adjacent to the spinal cord, or administration may be systemic, e.g., by intravenous administration. Dosage and frequency of dosing according to the methods disclosed herein may be adjusted as needed to affect the desired outcome in the subject. For example, treatment outcomes may be assessed for a particular subject may by monitoring the metabolic activity of myoblasts from blood samples taken from the subject, or by monitoring other features of the subject, such as increase in weight, improvement in muscle tone and muscle fitness, or other quality of life improvements. For example, improvement in the functional abilities as demonstrated by changes in the Spinal Muscular Atrophy Functional Rating Scale (SMAFRS) may indicate effectiveness of the methods disclosed herein. In some examples, the methods of treatment disclosed herein result in improvements in the SMAFRS functional rating, when the subject treated suffers from SMA.

### Combination Therapy for SMA

The present methods can be combined with another treatment for SMA. Genetic therapies have had some success preventing the necessity of permanent breathing support and the death of neonatal patients (type 1-2) such as Onasemnogene abeparvovec (Zolgensma®), an IV administered adeno-associated viral vector-based gene therapy, that delivers a copy of the survival of motor neuron 1 (SMN1) gene. Additionally, SMA neurorestorative agents, such as Nusinersen (Spinraza®), an intrathecally delivered antisense oligonucleotide (ASO) targeting the SMN2 gene, and Risdiplam (Evrysdi®), an oral survival of motor neuron 2 (SMN2)-splicing modifier, are available that slow or prevent motor neuron death due to SMA. Those available treatments differ by their mechanisms of actions on the disease and their means of administration, but can be combined with the presently claimed methods.

### Treatment of FSHD and Combination Therapy

Provided herein are methods for treating a subject (for example, a human subject) with FSHD. The methods may be utilized to treat (i.e., prevent, ameliorate, suppress, and/or alleviate) a motor impairment due to FSHD in the subject. The subject can have FSHD type 1 or FSHD type 2.

FSHD is the third most common inherited myopathy, with an estimated incidence of about 1:20,000. The disease is characterized by progressive weakness and atrophy of the facial and shoulder girdle muscles, which subsequently spreads to the abdominal and pelvic girdle muscles with highly variable expression. The genetic defect follows autosomal dominant inheritance and new mutations account for approximately 10% of recognized cases. FSHD type 1 is s the most common form of FSHD, accounting for 95 percent of cases. FSHD is caused by ectopic expression of the germline transcription factor double homeobox (DUX)4 gene in muscle cells. FSHD1 is associated with the deletion of a chromosomal tandem repeat called D4Z4 near the end of chromosome 4 at the 4q35 location. The D4Z4 region is a polymorphic variable number tandem repeat (VNTR) array consisting of 3.3 kilobase units, and each unit encodes for the DUX4 gene. Unaffected individuals have a chromosome 4 D4Z4 array that has a span of 11 to 150 contiguous units. In individuals with FSHD, this D4Z4 repeat array is contracted to a range between one to 10 contiguous units. The contraction of the D4ZA repeat array causes hypomethylation and relaxation of chromatin, which activates expression of the toxic DUX4 gene. Adjacent to the D4Z4 region, toward the distal end of the chromosome, lies a polyadenylation site that is part of the DUX4 gene and is required for stable expression of the gene in the most distal D4Z4 unit. Chromosome 4 comes in two alleles, called type 4qA and 4qB based on DNA variations distal to the D4Z4 repeat array. Only the 4qA variant in combination with the shortened D47A repeat region is associated with FSHD These subjects are amenable to treatment using the methods disclosed herein.

Facioscapulohumeral muscular dystrophy type 2 (FSHD2) accounts for approximately 5% of all cases of FSHD and describes patients without a D4Z4 repeat contraction on chromosome 4. Phenotypically FSHD2 shows virtually no difference from FSHD1 and both forms of FSHD arise via a common downstream mechanism of epigenetic de-repression of the transcription factor DUX4 in skeletal muscle cells. This results in expression of DUX4 and target genes leading to skeletal muscle toxicity. FSHD2 is a digenic disease and that mutations in the genes SMCHD1, DNMT3B, and more recently LRTF1, can cause FSHD2. These subjects are amenable to treatment using the methods disclosed herein.

The presently disclosed methods are of use with other treatment for FSHD. These include, but are not limited to exercise, diet modifications, surgical procedures, such as to stabilize the shoulder, and mechanical assistance. The presently disclosed methods can also be used in conjunction with other therapeutic agents, see for example, U.S. Published Patent application No. 2021/0038653, which disclosed the use of antibodies linked to inhibitors of DUX4, PCT Publication No. WO 2022/115745A1, which discloses the use of DUX4 antisense RNA, and U.S. Pat. No. 10,907,157, which discloses antisense agents and interference agents that reduce the expression n\of DUX4 and DUX4c.

FSHD may be treated by administering MBV, conditioned media, or myeloid-progenitor cells prepared as disclosed herein to the patient. Administration may be by systemic, e.g., intravenous administration, or administration may be directly to affected muscles, for example, by intramuscular administration. Dosage and frequency of dosing according to the methods disclosed herein may be adjusted as needed to affect the desired outcome in the subject. For example, treatment outcomes may be assessed for a particular subject by monitoring the metabolic activity of myoblasts from blood samples taken from the subject, or by monitoring other features of the subject, such as increase in weight, improvement in muscle tone and muscle fitness, or other quality of life improvements. For example, improvement in the functional abilities as demonstrated by changes in the FSHD compositive outcome measure (FSHD-COM) indicative of an improvement in quality of life indicators, or through measurements of muscle strength, leg function (timed walk test, time to ascend stair test, or time to get up from chair) may indicate effectiveness of the methods disclosed herein. In some examples, the methods of treatment disclosed herein result in improvements in the FSHD-COM functional rating, in a timed walk test, timed stair test, and/or time to stand test, when the subject treated suffers from FSHD.

### Promoting Skeletal Muscle Regeneration

Provided herein are methods for promoting skeletal muscle regeneration or repair in a subject in need thereof. A subject in need thereof has experienced muscle loss or damage. For example, the subject may have a muscle degenerative disease as disclosed herein contributing to muscle damage or loss. The methods provide for administering an effective amount of a composition comprising exogenous matrix bound nanovesicles (MBV) derived from extracellular matrix, wherein the MBV do not express CD63 and CD81 or are CD63^{lo}CD81^{lo} and wherein the MBV do not contain alkaline phosphatase.

Subjects administered MBV may demonstrate increases in satellite cells and/or myoblasts in muscle tissue, in particular, in a muscle tissue where regeneration or repair is desired. Subjects administered MBV may demonstrate increases in myofibers in the muscle tissue where regeneration or repair is desired. The MBV may be administered systemically. The MBV may be administered locally to the muscle where regeneration or repair is desired. The method can include measuring muscle regeneration, such as measuring increases in satellite cells and/or myoblasts in a sample from the subject.

While observance of such cellular changes in the muscle tissue can be examined by biopsy and staining of tissue, increase in muscle weight is another metric that can be used to determine that muscle regeneration is occurring. Increases in muscle weight may be determined by calculating body composition through, e.g. bioelectrical impedance, dual-energy X-ray absorptiometry (DEXA/DXA) scan, or magnetic resonance imaging (MRI), and determining the proportion of the body weight that is fat, muscle, water, or hone.

The level of muscle regeneration or repair achieved in a subject from administration of MBV can also be assessed functionally by using strength testing. For example, peak torque (PT), rate of torque development (RTD), or Average Torque (AT) over a single contraction can be used to assess muscle function during isometric contraction of the muscle. Assessments can be made both before and after treatment to assess muscle function, where increases in muscle function after treatment as compared to before treatment are indicative of regeneration and repair of skeletal muscle. According to the invention, a subject administered MBV experience a 1%, 2%, 3%, 4%, 5%, 6%, 7%, 8%, 9%, 10%, 15,%, 20% or more increase in PT, RTD, or AT after being treated with MBV, as compared to before treatment. The increase may be measured 1 week, 2 weeks, 1 month, 2 months or 3 months after receiving administration of MBV.

The skeletal muscle experiencing regeneration or repair may be the quadriceps, biceps, gastrocnemius, hamstrings, gluteus maximus, triceps, deltoid, latissimus dorsi, or trapezius, for example. The subject may be human.

According to the invention, a subject administered MBV experience a 1%, 2%, 3%, 4%, 5%, 6%, 7%, 8%, 9%, 10%, 15,%, 20% or more increase in muscle mass after being treated with MBV. For example, this increase may be measured after about 1 month, about 2 months, about 3 months, about 4 months, about 5 months, or about 6 months after beginning treatment with MBV. The increase is compared to the muscle mass of the subject prior to beginning treatment with MBV.

MBV may be administered chronically (e.g., indefinitely as long as a need for muscle regeneration exists) or for a limited period of time until a desired level of muscle regeneration is achieved.

For example, a subject is administered about 1×10¹ to about 1×10²⁰ MBV per kg of body weight per administration. For example, a subject is administered about 1×10⁶ to about 1×10²⁰ MBV/kg of body weight, such as about 1×10⁶ to about 1×10¹² MBV per kg of body weight per administration. In some examples, a subject is administered about 1×10⁶ to about 1×10¹⁹ MBV, about 1×10⁶ to about 1×10¹⁸ MBV, about 1×10⁶ to about 1×10¹⁷ MBV, about 1×10⁶ to about 1×10¹⁶ MBV, about 1×10⁶ to about 1×10¹⁸ MBV, about 1×10⁶ to about 1×10¹⁷ MBV, about 1×10⁶ to about 1×10¹⁶ MBV, or about 1×10⁶ to about 1×10¹² MBV per administration. In other examples, a subject is administered about 1×10⁷ to about 1×10¹¹ MBV per kg of body weight per administration. In another example, a subject is administered 1×10⁷ to 1×10⁸ MBV per kg of body weight per administration. In another embodiment, a subject is administered 1×10⁷ to 1×10¹⁰ MBV per kg of body weight per administration. In another embodiment, a subject is administered 1×10⁹ to 1×10¹⁰ MBV per kg of body weight per administration. In another embodiment, a subject is administered 1×10⁶ to 1×10⁸ MBV per kg of body weight per administration. In another embodiment, a subject is administered 1×10⁷ to 1×10⁹ MBV per kg of body weight per administration. In another embodiment, a subject is administered 1×10⁸ to 1×10¹¹ MBV per kg of body weight per administration. In another embodiment, a subject is administered 1×10⁹ to 1×10¹¹ MBV per kg of body weight per administration. In another embodiment, a subject is administered 1×10¹⁰ to 1×10¹¹ MBV per kg of body weight per administration. In another embodiment, a subject is administered 1×10¹¹ to 1×10¹² MBV per kg of body weight per administration. In another embodiment, a subject is administered 1×10⁶ to 1×10¹⁴ MBV per kg of body weight per administration. In another embodiment, a subject is administered 1×10¹² to 1×10¹⁴ MBV per kg of body weight per administration. In one embodiment, administration of MBV according to any of the aforementioned amounts is by systemic administration. For example, in one embodiment, the administration is intravenous. In another embodiment, the administration is by bolus subcutaneous injection. The administration may be, for example, intramuscular, for example, into the muscle where regeneration is desired. The intramuscular injection may be a single bolus injection or divided and introduced at multiple sites in the muscle in need of repair or regeneration.

The MBV may be administered once per day, once per week, biweekly, every two weeks, once per month, or as needed to achieve the desired level of muscle regeneration or repair.

The following examples are provided to illustrate particular features of certain aspects of the disclosure, but the scope of the claims should not be limited to those features exemplified.

### EXAMPLES

Matrix Bound Nanovesicles (MBV) are extracellular vesicles present in the extracellular matrix (ECM) of all animal tissues, including muscle. MBV have been shown to regulate the phenotype of macrophages by promoting pro-remodeling phenotypes and downregulating inflammation. Many other cell types such as mesenchymal stem cells, fibroblast and neural progenitors take up MBV, and have biological responses to MBV. The following examples show that MBV have a direct effect on FSHD primary muscle cells and a homeostatic effect on macrophages. The effect on the secretome of neural progenitor cells and microvessel-endothelial cells is also shown herein. In addition, MBV were demonstrated to have an effect in animal models of FSHD and spinal muscle atrophy. The results support the therapeutic utility of MBV as a treatment for muscle wasting conditions disclosed herein.

### Example 1

### Materials and Methods

MBV isolation: MBV were isolated from porcine urinary bladder (UBM) previously decellularized. MBV isolation was carried out by liberase (collagenases and dispase enzyme cocktail) digestion of UBM, ultracentrifugation and size exclusion chromatography (SEC). Isolated MBV were quantified by nanotracking analysis (NTA) and stored at −20 °C before use.

FSHD2 primary cell treatment: Cell culture was carried out under sterile conditions, and cells were incubated at 37 °C and 5% CO₂ conditions for all the experiments. Myoblasts from healthy and Facioscapulohumeral Muscular Dystrophy (FSHD)-diagnosed patients were cultured in proliferation medium (Ham's F-10, 20% FBS, 1% PS) in 96-well plates. At 80% confluency, cells were treated with different doses of MBV (1×10⁹, 1×10¹⁰ and 1×10¹¹ particles/ml) for 24 hours. Cells were then washed in PBS and incubated with 10% ALAMARBLUE™ to measure the metabolic activity.

Macrophage secretome generation: Macrophages were isolated from C57BL/6 mice hone marrow and differentiated for 7 days as per standard laboratory protocol. Differentiated macrophages were then treated with 1×10¹¹ MBV/ml in complete medium (DMEM high glucose 10% FBS and 1% PS) for 24 hours, using LPS and IL-4 treated macrophages as M1 and M2 controls respectively. After 24 hours, medium was aspirated, cells were washed with PBS, and blank DMEM (0% FBS) was added. Cells were incubated for 6 hours and medium with the macrophages' secretome was collected, filtered and stored at −20 °C.

Human microvascular endothelial cells (HMEC-1) culture and angiogenesis assay: HMEC-1 were grown in MCDB131 medium with 10% FBS and 1% PS and seeded in 24-well plates until they reached 90% confluency. Then, a straight scratch was performed on the cell monolayer with a 20 μl pipette-tip, cells were washed with PBS and treated with a 1:1 mixture of MCDB131 medium with 20% FBS and the macrophage-derived secretome (MBV, M1, and M2). Pictures of the scratch area were taken at 0 h, 2 h, 4 h and 12 h. The pictures were analyzed with ImageJ software to calculate the scratch area, which decreased with cells, indicating cell migration.

For metabolic activity analysis, cells were seeded in 48-well plates. When they reached 80% confluence, HMEC-1 cells were cultured with the macrophage secretome and complete medium in a 1:1 mixture for 24 and 72 h. Then, the medium was replaced with 10% ALAMARBLUE™ and incubated for 3 hours. After incubation, fluorescence (530 nm excitation, 590 nm emission) was recorded and related to the metabolic activity.

Mouse neuroblastoma cells treatment and differentiation: N1E-155 neuroblastoma cells were cultured in DMEM with 10% FBS and 1% PS. Cells were seeded in 48-well plates until they reached 80% confluency. Then, cells were cultured with the macrophage secretome and complete medium in a 1:1 mixture for 24 and 72 h. The medium was replaced with 10% ALAMARBLUE™ and incubated for 3 hours. After incubation, fluorescence (530 nm excitation, 590 nm emission) was recorded and related to the metabolic activity.

For differentiation analysis, cells were seeded at 20% confluency with macrophage-secretome and complete medium in a 1:1 mixture for 24 h and 72 h. At each time point, representative pictures of cells were taken and images were analyzed with software CellProfiler. Single cell features such as roundness, eccentricity and Max Feret diameter were measured and related to differentiation stages of neuroblastoma cells.

Statistical analysis: Data was presented as average t standard deviation. Figures and statistical analysis were carried out the Prism GraphPad Software. One-way ANOVA and Tukey's post-hoc tests were performed to assess statistical differences in metabolic activity assays, whereas two-way ANOVA and uncorrected Fisher's post-hoc assays were used to assess statistical differences in the scratch and differentiation assays.

### Example 2

### MBV-Direct Effect on FSHD-Cells

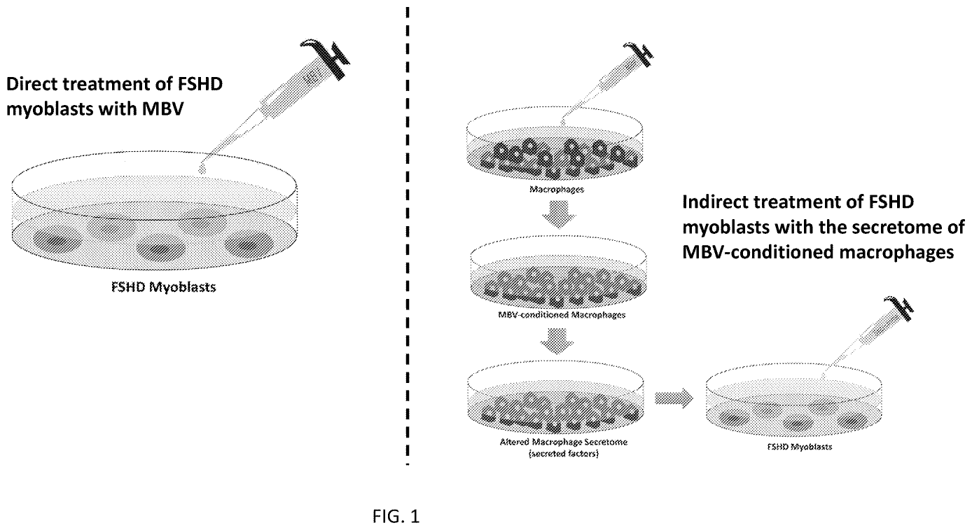

The metabolic activity of healthy and FSHD2 patient-derived myoblast showed a linear increase in metabolic activity with increasing doses of MBV (FIG. 1). These results demonstrated that MBV increased mitochondrial respiration and/or cell proliferation of both healthy and affected myoblasts. Measurements were taken 24 hours after being treated with different doses of the MBV.

### Example 3

### MBV-Treated Macrophage Secretome Effect on HMEC-1 Cells

The macrophage derived secretome from all groups slightly increased the metabolic activity of HMEC-1 cells after 24 hours (FIG. 2, left panel), while only the MBV-derived secretome increased the metabolic activity of endothelial cells after 72 hours (FIG. 2, right panel).

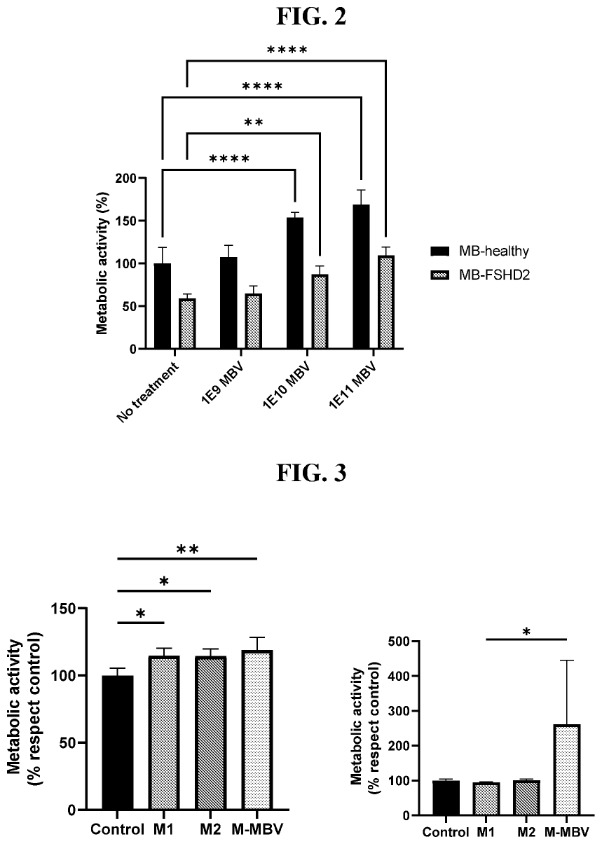

A scratch assay showed no differences between treatments after 2 and 4 hours. However, at 12 hours, cells treated with the MBV-secretome showed a higher migration than the rest of the groups (FIG. 3). These results indicate that the secretome derived from MBV-treated macrophages induces an activation effect on endothelial cells, an effect that is associated with angiogenesis.

### Example 4

### MBV-Treated Macrophage Secretome Effect on NIE-155 Cells

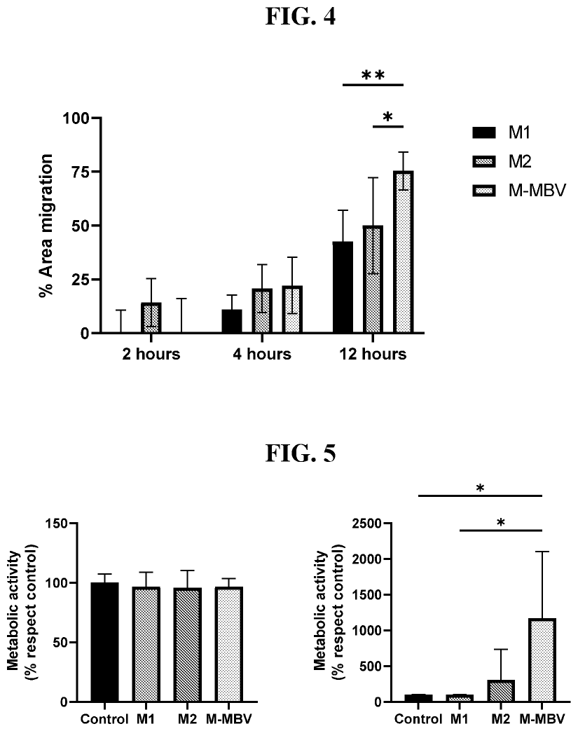

The macrophage derived secretome from all groups (M1, M2, MBV) showed no difference on the metabolic activity of NIE-155 cells after 24 hours, while the M2 and MBV-derived secretome increased the metabolic activity of neuroblastoma cells after 72 hours. This effect was significantly more marked in when cells were treated with the MBV-secretome (FIG. 4).

Neuroblastoma cells showed changes in morphology (roundness, eccentricity ratio and Feret diameter) after 72 h when treated with M0, M1 and M2 macrophage secretomes (FIGS. 5A-5B). However, when treated with MBV-secretome, roundness and Feret diameter showed no changes after 72 hours. As the changes in morphology of NIE-155 cells are highly related to their level of terminal differentiation, these results suggested that the MBV-secretome from macrophages preserved the sternness of neural progenitors in vitro.

Overall, the results support the use of MBV directly and of MBV-treated macrophages or the secretome derived from MBV-treated macrophages for treatment of muscular dystrophies. The results showed that MBV directly interact with myoblasts, increasing their metabolic activity. In the case of FSHD2 myoblasts, because FSHD is highly related to metabolic malfunction and oxidative stress, the increase of metabolic activity implies an improvement at metabolic level of affected cells. The results also demonstrated that macrophages, when treated with MBV, are able to produce a secretome that promotes activity of endothelial cells and stemness of neural progenitor cells. As macrophages are the main regulators of the innate immune response and regeneration, the effect of MBV on macrophages in vivo can promote muscle regeneration in this dystrophy.

### Example 5

### Uptake Analysis Measured by Flow Cytometry

This analysis was undertaken to determine whether MBV accumulate in the bone marrow, interact with myeloid progenitors, and potentially reprogram or alter the phenotype of myeloid innate cells.

A panel of markers was used to assess the interaction of MBV with different cell types in the bone marrow. To do so, before staining, bone marrow from BalbC mice were flushed, strained through a 40 μm strainer and exposed to 1×10¹¹ CFSE-tagged MBV/ml in vitro 24 h and 3 h before flow cytometry.

Cells were then tagged with the following panel of markers for different immune cell subtypes:

| Antibody | Target |
|---|---|
| CSFE | MBV |
| CD11b | Myeloid Cells |
| CD34 | Hematopoetic/myeloid progenitor cells |
| FcRgI | Myeloid cells |
| CD64 | Progenitors |
| F4/80 | Macrophages |
| CD4 | T-cells |
| CD335 | NK cells |
| Ly6G | Neutrophils |

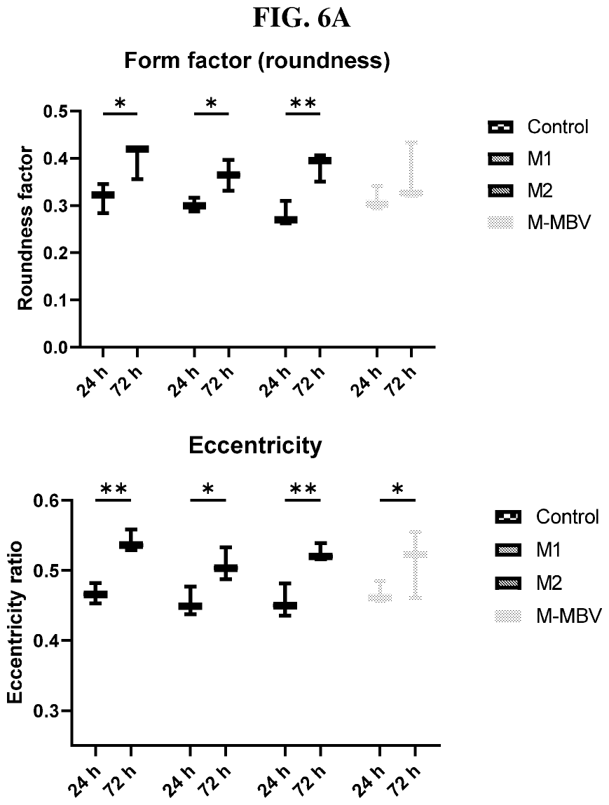

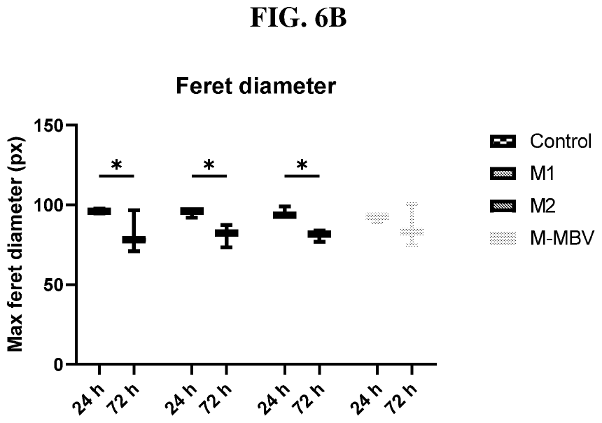

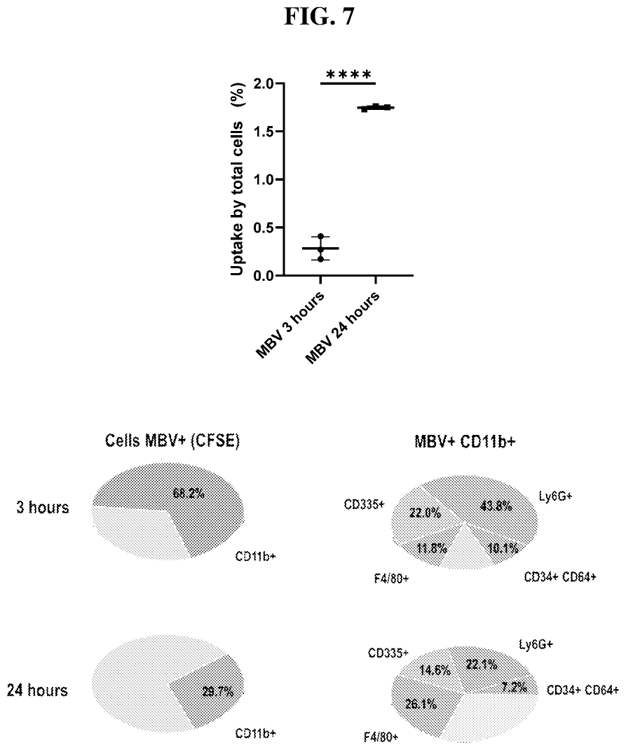

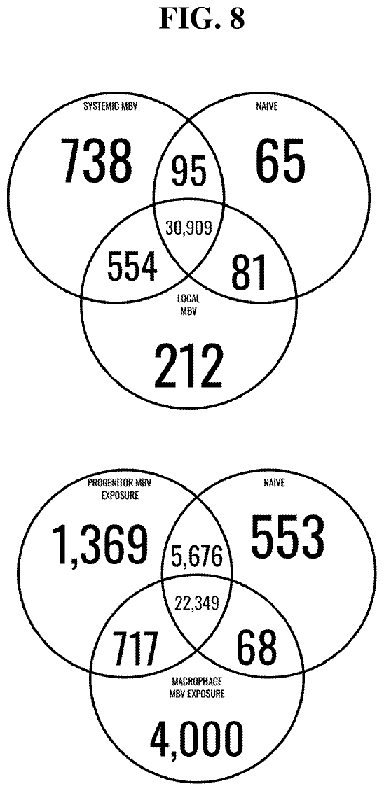

Flow cytometry showed that cells from bone marrow take up MBV in vitro. After 3 hours, the vast majority of these cells were myeloid cells, whereas more cells from other cell lineages were able to uptake MBV after 24 hours. See FIG. 6. Within these myeloid cells, a considerable percentage were myeloid progenitors (CD34 and CD64 positive), whereas the other major myeloid cells that uptake MBV are neutrophils (Ly6G positive) and macrophages (F4/80 positive).

### Example 6

### ATAC-Seq Analysis

An Assay of Transposase Accessible Chromatin Sequencing was carried out on bone marrow flush in vitro, differentiated macrophages in vitro, or after systemic and local injection to determine if treatments by MBV affect the accessibility of chromatin within the whole genome, and translate into stable phenotypic changes. If such changes occur in progenitor cells, it could indicate a stable change in the subsequent cell lineages of the innate immune response.

ATAC-seq analysis showed that myeloid progenitors and terminally differentiated macrophages are differently affected by MBV in vitro (FIG. 7), which translated in upregulation of 28 genes uniquely in MBV-treated progenitors, 5917 in MBV-treated macrophages, while 168 of these upregulated genes were shared (Venn Diagram). These results suggest that MBV elicit stable phenotypic changes in progenitors that can be inherited descendent myeloid lineages.

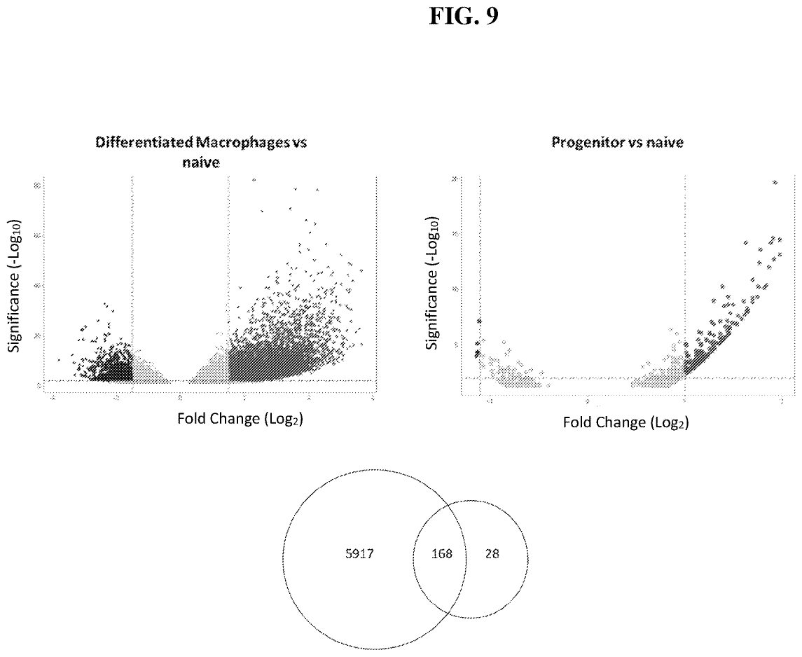

The distribution of statistically significant regulated genes at the epigenetic level in the different treatments (macrophages or hone marrow) in vitro is shown in FIG. 9. In the Venn diagram, the large circle corresponds to the dots on the left side of the graph, and the small circle corresponds to the dots on the right side of the graph.

### Example 7

### Accumulation of MBV in Bone Marrow

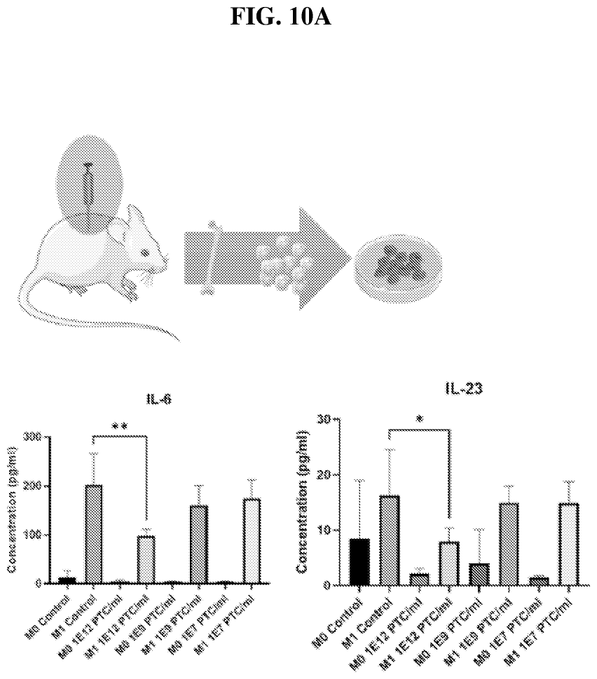

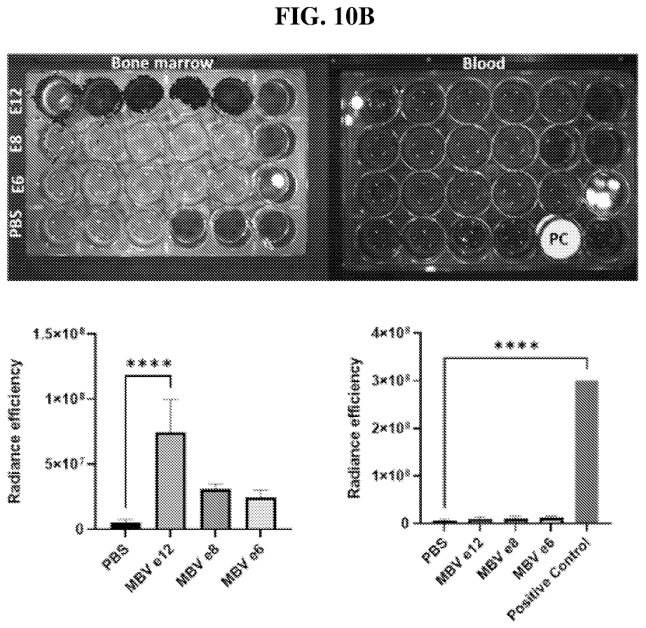

DiD-tagged MBV were injected in Balb/c mice 3 h and 24 h. At the different time points, bone marrow was flushed and the presence of fluorescent signal in the marrow was measured ex vivo using an IVIS equipment. Er vivo analysis showed that MBV accumulated in the bone marrow after systemic injection, generating a significant signal. In addition, this accumulation was linked to a change in phenotype (FIGS. 10A-B).

The data from Examples 5, 6, and 7 show that MBV accumulate in the bone marrow and interact with different myeloid cell types, including progenitors. This interaction is linked to changes in chromatin accessibility at epigenetic level, which can be related to changes in the phenotype. These results indicate that MBV could be used for stable modulation of myeloid lineages' immune response.

### Example 8

### Results in Animal Models of FSHD and SMA

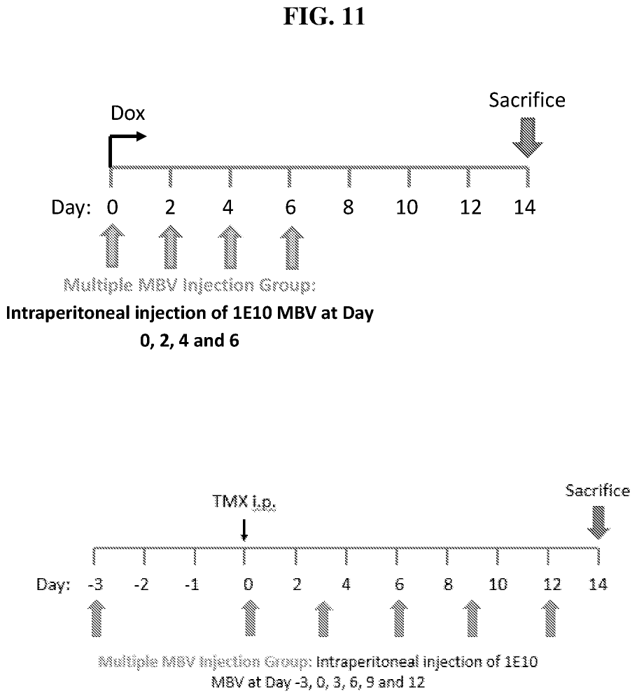

The effect of MBV was tested in two animal models of FSHD, see FIG. 11. These animal models are disclosed in DeSimone et al., Disease Models & Mechanisms (2020) 13, dmm046904. doi:10.1242/dmm.046904, 2022, incorporated herein by reference. The effect of MBV was also tested in an animal model of SMA, see Feng et al., Human Mol. Gen. 25(5): 964-975, 206, incorporated by reference herein.

For these studies, the SMA intermediate model was generated by treating delta7 mice with SMN-C3, an SMN-upregulating compound. Mice were treated with SMN-C3L (3 mg/kg I.P., daily) starting postnatal day (PND) 1. At PND21, mice were injected with either MBV in saline, MBV in hydrogel or PBS, intramuscularly, into both gastrocnemius, both tibialis anteriors, both quadriceps, both triceps and both masseters. Muscle function was measured at PND50, then the mice euthanized, and tissues collected.

For FSHD, a first study was performed using the iDUX4 pA-HSA mouse model. This model used doxycycline (dox) to induce expression of the iDUX4 pA transgene via a muscle fiber-restricted reverse tetracycline-controlled transactivator (rtTA). (Bosnakovski et al., J. Clin. Invest., 2020 13(5): 2465). DUX4 expression was induced by feeding four-week-old female iDUX4 pA-HAS mice dox chow on Day 0 and maintained for the duration of the study to simulate a “moderate” DUX4 environment. Animals were treated with MBV or saline according to the protocol shown in FIG. 11 (top).

In a second animal model, the FLExDUX4(+TMX) mouse model was used which permits TMX-inducible cre-expressing to permit spatiotemporal control of DUX4-fl expression during development or in adults. (Jones & Jones, 2018, PLoS One, 13(2):e0192657). Animals were treated with MBV or saline according to the protocol shown in FIG. 11 (bottom).

The animals were graded on a severity scoring scale where normal is assigned a score of 1; 2 reflects increased but minimal inflammatory cells with some satellite cell activation; 3 represents myonecrosis, including satellite cell activation, including myoblasts, abundant inflammatory cells and loss cytoplasmic integrity; and 4 is the most severe with demonstration of disseminated myonecrosis, and abundant inflammatory cells, including myoblasts, fibrosis, and loss of cytoplasmic integrity.

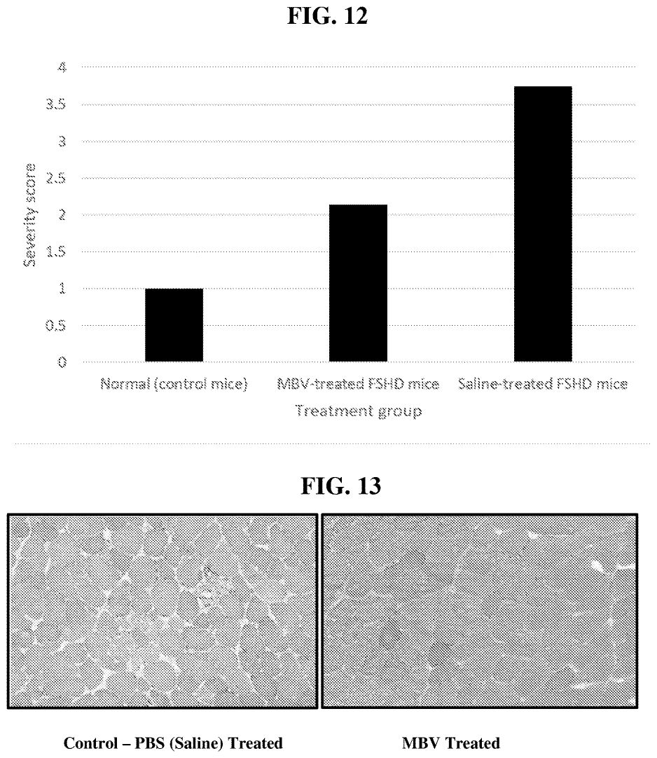

As shown in FIG. 12, FSHD animals (TMX induced) treated with MBV had an average severity score of about 2.2, whereas FSHD animals treated with saline only had an average severity score of about 3.7, demonstrating that MBV have a positive impact in reducing FSHD severity score as compared to no treatment.

As shown in the exemplary tissue images found in FIG. 13 of skeletal muscle samples taken from PBS (saline) TMX-treated mice (left) and MBV treated mice (right), the PBS treated animal shows shrunken skeletal muscle fibers, focal areas of necrosis, and increased numbers of inflammatory cells between fibers. In contrast, the exemplary muscle fiber sample from an MBV treated animal shows normal size fibers and evidence of activated satellite cells at the periphery of the skeletal muscle fibers. Accordingly, MBV have a positive impact inducing phenotypic changes indicative of healthy muscle cells in FSHD mice.

An SMA intermediate model generated by treating delta7 mice with SMN-C3, an SMN-upregulating compound was used to study the impact of MBV on SMA. Mice were treated with SMN-C3L (3 mg/kg I.P., daily) starting PND1. At PND21, mice were injected with either MBV in saline, MBV in hydrogel or PBS, intramuscularly, into both gastrocnemius, both tibialis anteriors, both quadriceps, both triceps and both masseters. (FIG. 15). Muscle function was measured at PND50, then the mice euthanized, and tissues collected. Of PBS treated control mice, the mean FSHD score was 3.3 (n=7). Of MBV treated mice, the mean FSHD score was 2.5 (N=12). Of mice treated with MBV contained within an extracellular matrix hydrogel, the mean FSHD score was 2.2 (n=11).

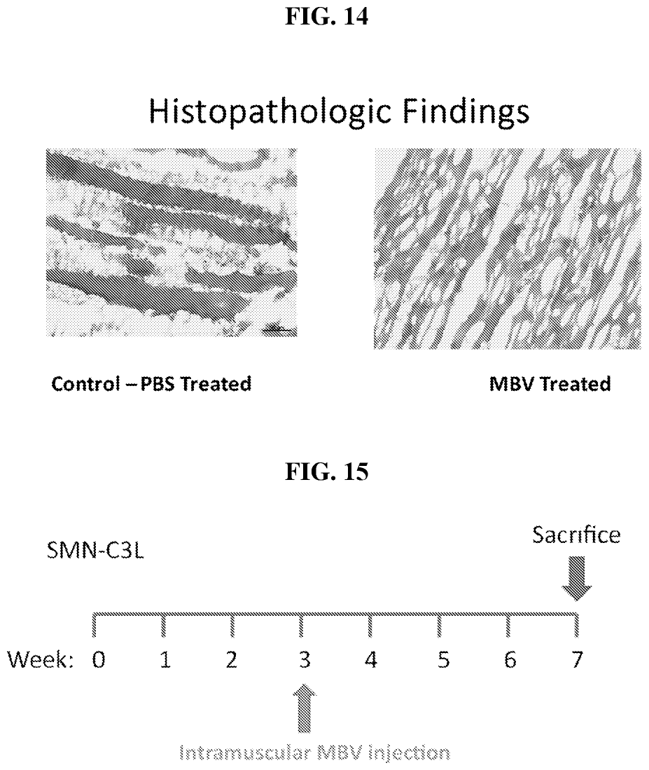

As shown in FIG. 14, control SMA animals showed marked atrophy of fibers (longitudinal sections) with abundant inflammatory cells between fibers and areas of muscle necrosis. In contrast, MBV treated animals showed muscle atrophy but diminished inflammatory cells compared with PBS treated animals. There is also less damage to the individual fibers. Accordingly, MBV may be used to mitigate the impacts of SMA on muscle fibers.

### Example 9

### MBV do not Express the Common Exosomal Markers CD63 or CD81

EXO-CHECK™ Exosome Antibody Array (System Biosciences) was used to compare the presence or absence of common exosomal markers on murine exosomes, murine bone marrow bone matrix vesicles (Bone MV), and murine matrix bound nanovesicles (MBV). The results are shown in FIG. 15A and clearly show that MBV are virtually devoid of the canonical and well accepted markers of exosomes such as CD63 and CD81. In addition, the other signaling molecules identified in FIG. 15A were absent in MBV, whereas they were present to either moderate or high degrees in hone microvesicles and exosomes. A densitometry plot of the expression values is shown in FIG. 15B.

The data show that while exosomes and Bone MV share a similar expression profile with moderate to high expression of these markers, MBV are significantly different in the expression of these EV markers. For example, MBV are also virtually devoid of, i.e., have “low” or “undetectable” levels of one or more of EpCAM, ANXA5, TSG101, FLOT1, ICAM1, and ALIX as indicated by blank wells as compared to the presence of dark rings or solid dark spots in the same positions on the exosome wells and by the relative expression levels of these markers as shown in the graph in the bottom panel. Bone MV also have higher levels of expression of all of these markers as compared to MBV as shown by the dark spots on the wells and by the relative expression levels of these markers as shown in the graph in the bottom panel.

In one embodiment, MBV have low or undetectable levels or one or more of CD63, CD81, EpCAM, ANXA5, TSG101, FLOT1, ICAM1, and ALIX as compared to a positive control of the EXO-CHECK™ Exosome Antibody Array. In one embodiment, MBV have low or undetectable levels of one or more of CD63, CD81, EpCAM, ANXA5, TSG101, FLOT1, ICAM1, and ALIX as compared to an exosome, for example, a plasma exosome. In one embodiment, MBV have low or undetectable levels or one or more of Cd63, CD81, EpCAM, ANXA5, TSG101, FLOT1, ICAM1, and ALIX as compared to a bone MV. In one embodiment, MBV are characterized by low or undetectable levels of ANXA5, TSG101, and ICAM1 as compared to exosomes or bone MV. In one embodiment, MBV are characterized by low or undetectable levels of CD81, CD63, ANXA5, TSG101, and ICAM1 as compared to bone MV or plasma exosome.

### Example 10

### MBV have Low or No Expression of Bone MV Markers

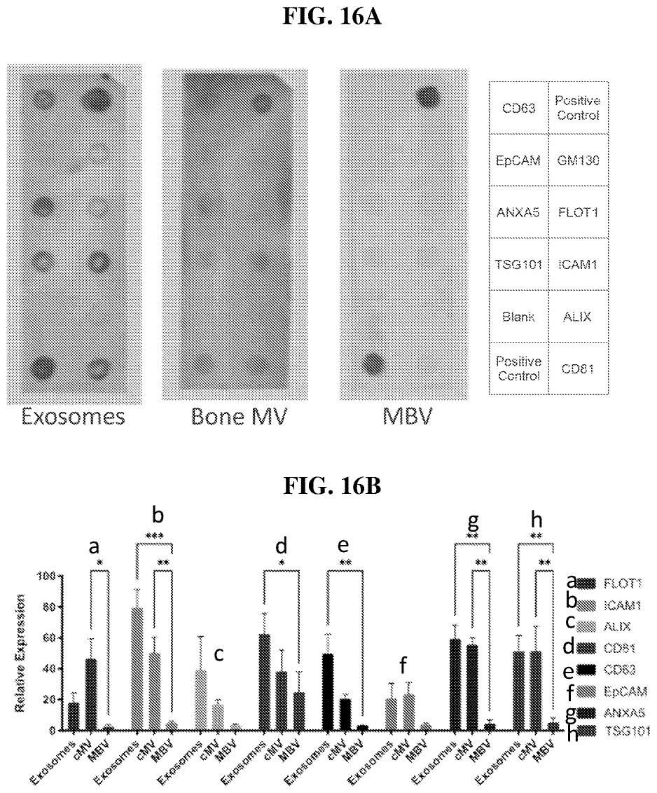

The expression of the Bone MV markers Annexin V, and Tissue Non-specific Alkaline Phosphatase (TNAP) were evaluated by Western blot analysis. The results are shown in FIG. 16. Lysate prepared from 1711A Cells was used as a positive control. The results of this experiment show that matrix bound nanovesicles (MBV) are devoid of any expression of both markers of bone microvesicles, TNAP and Annexin V. Plasma exosomes do express Annexin V, but do not express TNAP. These results clearly distinguish MBV from both exosomes and bone microvesicles

### Example 11

### MBV have a Differential Immunomodulatory Effect Compared to Exosomes or Bone MV

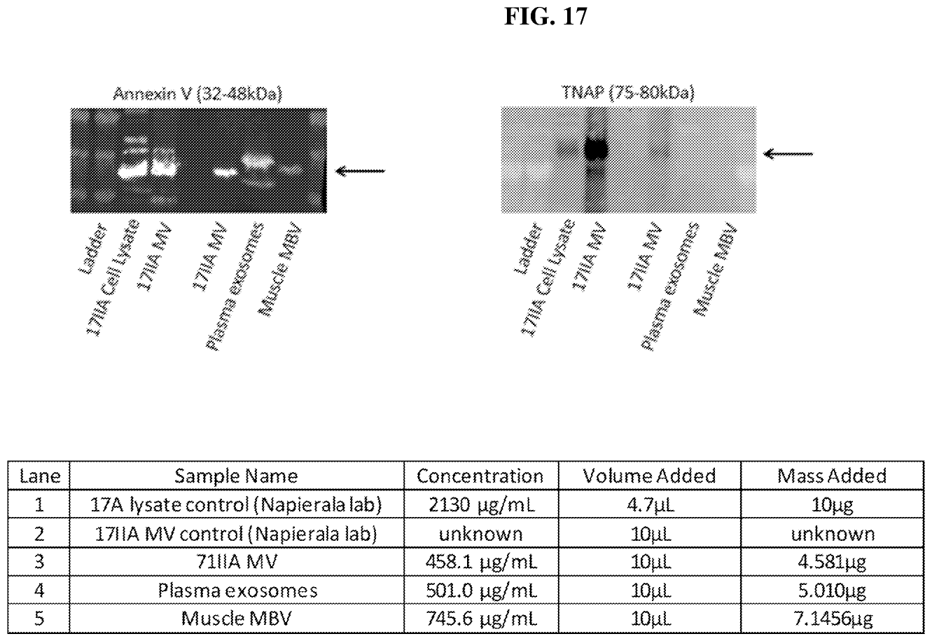

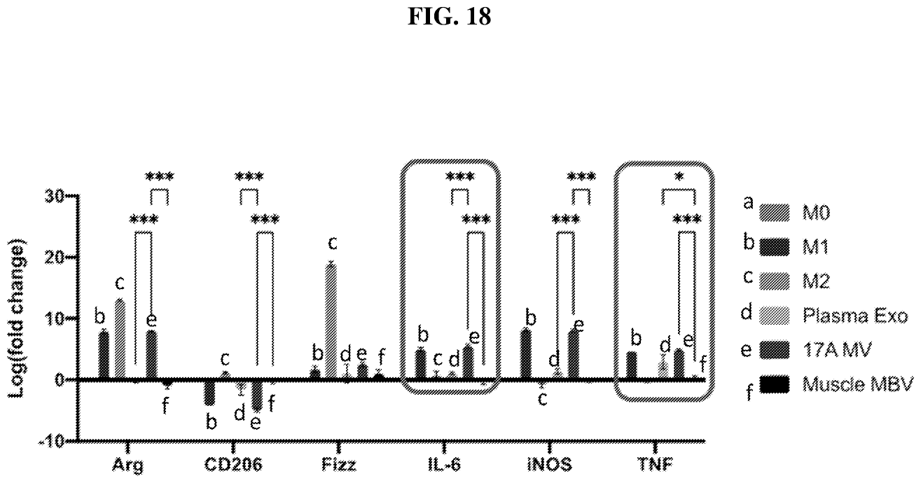

Bone Marrow-Derived Macrophages (BMDM) harvested from mice were untreated (M0) or treated with the following test articles for 24 hours: IFNγ+LPS to induce an M1 phenotype (M1), IL-4 to induce an M2-like phenotype (M2), Exosomes derived from plasma, Bone MV derived from 17A cell, or MBV isolated from muscle. After treatment, the fold change in the expression of the indicated genes was evaluated by qPCR. The results, shown in FIG. 17, show the downregulation of the pro-inflammatory markers IL-6 and TNF-α by MBV are clearly distinguished from the downregulation of the same two inflammatory mediators by exosomes and hone microvesicles. MBV had a potent anti-inflammatory effect; whereas exosomes and bone microvesicles did not.

### Example 12

### D2.mdx Mouse Study

### Materials and Methods:

Preparation of urinary bladder matrix (UBM): UBM was prepared as previously described (Mase V J, et al. Orthopedics. 2010; 33(7):511). Porcine urinary bladders from market-weight animals were acquired from Tissue Source, LLC. Briefly, the tunica scrosa, tunica muscularis externa, tunica submucosa, and tunica muscularis mucosa were mechanically removed. The luminal urothelial cells of the tunica mucosa were dissociated from the basement membrane by washing with deionized water. The remaining tissue consisted of basement membrane and subjacent lamina propria of the tunica mucosa and was decellularized by agitation in 0.1% peracetic acid with 4% ethanol for 2 hours at 300 rpm. The tissue was then extensively rinsed with PBS and sterile water. The UBM was then lyophilized and milled into particulate using a Wiley Mill with a #60 mesh screen.

Isolation of Matrix Bound Nanovesicles: MBV were isolated from laboratory produced porcine urinary bladder matrix (UBM) by enzymatic digestion with Liberase TL (highly purified Collagenase I and Collagenase II) in buffer (50 mM Tris pH7.5, 5 mM CaCl₂, 150 mM NaCl) for 24 h at room temperature on an orbital rocker. Digested ECM was then subjected to centrifugation at 10,000×g (30 min) to remove ECM debris. The clarified supernatant containing the liberated MBV was then centrifuged at 100,000×g (Beckman Coulter Optima L-90K Ultracentrifuge) at 4 °C for 2 hr to pellet the MBV. MBV were then resuspended in IX PBS and stored at 4 °C until further use.

The D2.mdx mouse model: This study used male mdx mice of the DBA/2J (D2.mdx; Jax #013141), see Sci Rep 10, 14070 (2020). Mice were randomly assigned into groups 1) saline treatment, and 2) MBV treatment. 100 μl containing 4.2×10\hat{ }⁹ of MBV were administered via intraperitoneal injection on days 1, 3, and 5, and then once weekly thereafter. Body weight was recorded weekly.

Isometric torque measurement: Functional analysis was performed by measuring isometric torque production of the gastrocnemius at weeks 1 and 7, see Tissue Eng Part A. 2018 January; 24(0-2):34-46. Animals were anesthetized while the hind limb was stabilized by platform supports with the foot in the flexed position. Muscles were stimulated at eight different frequencies (25-200 Hz) with a 2-min rest period between each frequency. Twitch contraction and tetanic contraction were analyzed using a Dynamic Muscle Analysis program and data were normalized to the animals' weights.

Histological Evaluation: Animals were sacrificed at week eight. Tissues were collected and fixed in neutral-buffered formalin, and paraffin embedded. Hematoxylin and Eosin (HE) and Masson's Trichrome staining was performed to evaluate the degenerative and/or regenerative areas, which were identified by the presence of inflammatory cells and muscle fiber necrosis.

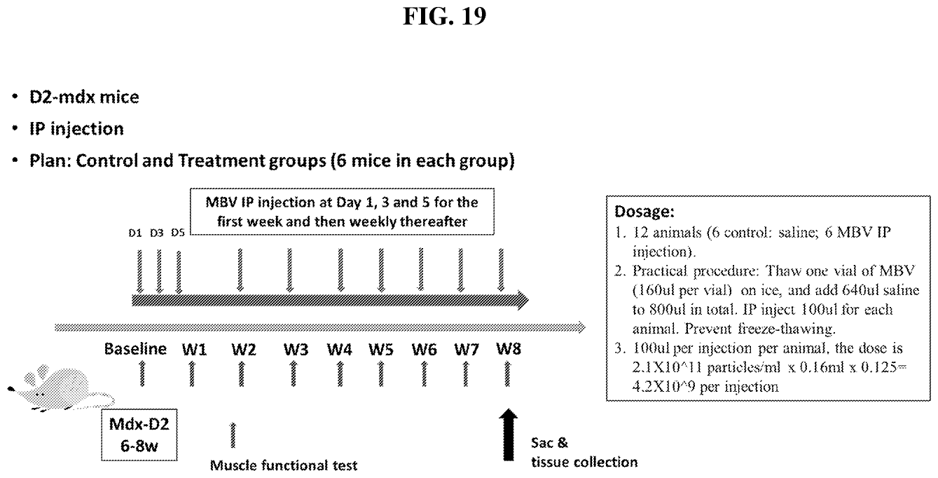

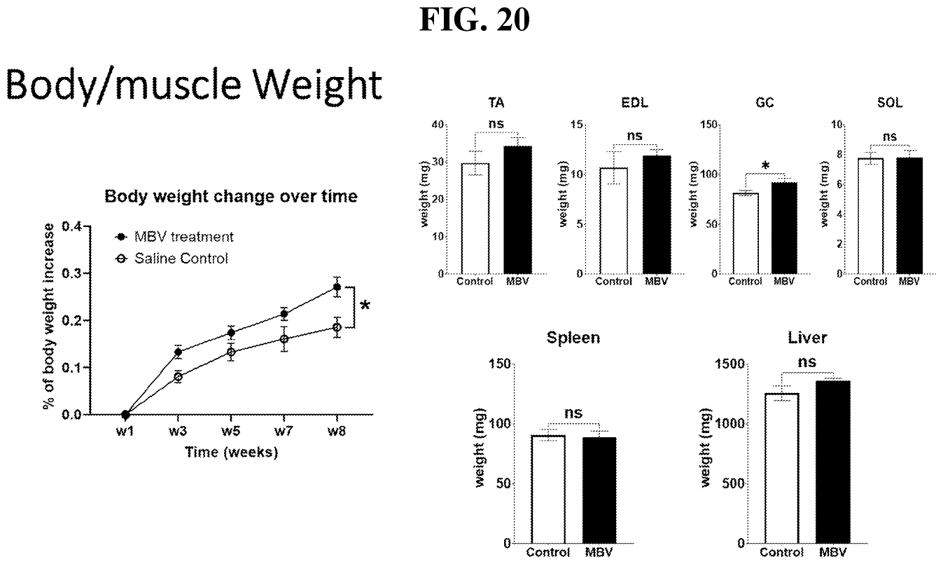

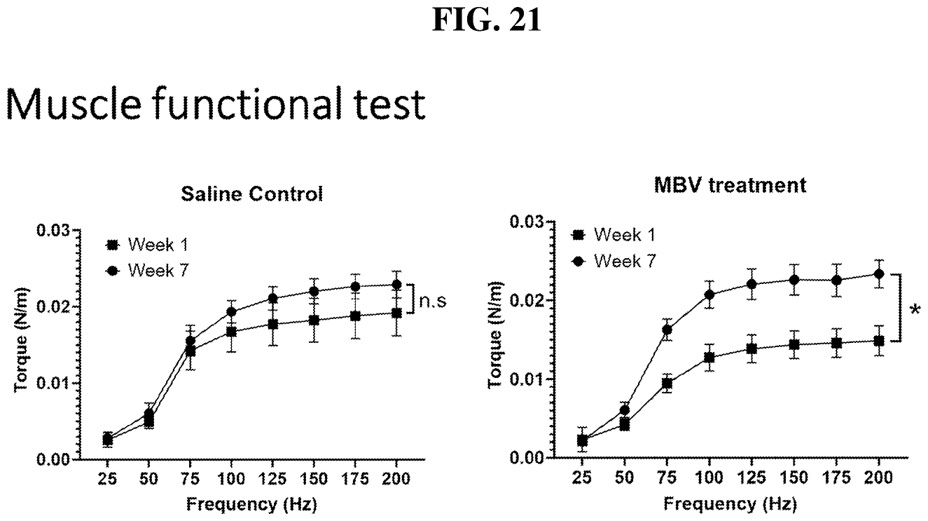

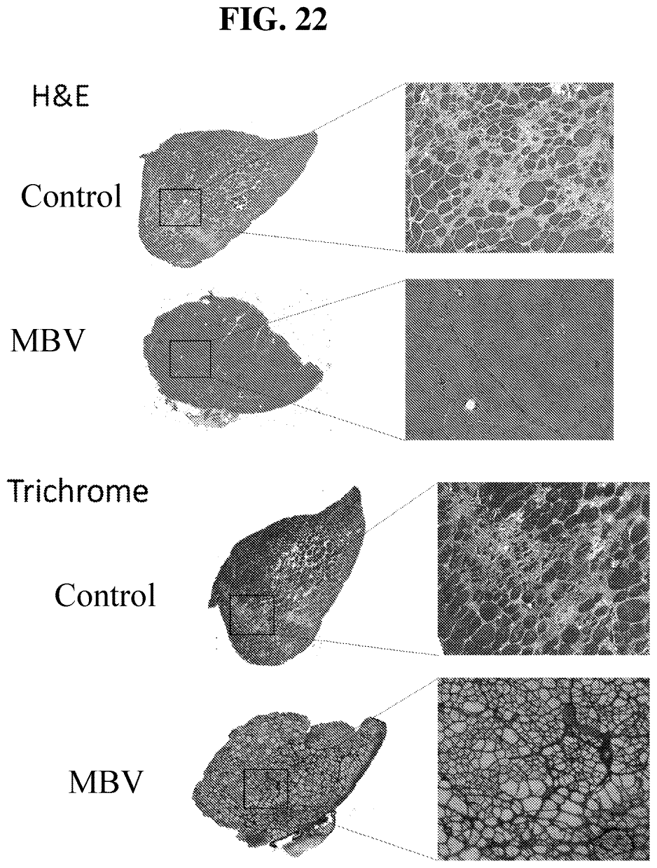

Results: The study protocol is shown in FIG. 19. Body and muscle weights were evaluated for the two treatment groups, see FIG. 20. Results from muscle function tests are shown in FIG. 21. Histological evaluation is shown in FIG. 22.

The results strongly suggest that MBV can mitigate the progression of DMD. The D2.mdx model is a severe version of the disease, and the use of the MBV provide unexpectedly superior efficacy. As shown in FIG. 20, the weight of the gastrocnemius in MBV treated animals was statistically significantly greater than in control animals, and for the other muscles, the trend was toward greater mass for those muscles in MBV treated animals as compared to controls, suggesting that MBV contribute to muscle regeneration and repair in the DMD model. As shown in FIG. 21, MBV treatment yielded muscles with statistically significant increases in torque production as compared to controls. Torque is a quantitative measure of muscle strength. Theses results are attributed to more muscle mass in the MBV treated group rather than greater strength for each muscle fiber. Increased torque over control animals suggests that MBV contributed to muscle repair and regeneration.

Further, the histology shows decreased muscle necrosis which, without being bound by theory, can be the result of decreased proinflammatory actions and/or an effect upon muscle cell anabolism in the MBV treated animals as compared to controls. Further, and surprisingly, satellite cell activation including the presence of myoblasts were observed in the MBV treated tissue, indicative of a cellular environment that promotes muscle regeneration and repair. These were not seen in the control tissue. This suggests that MBV can promote regeneration and repair of skeletal muscle tissue

It will be apparent that the precise details of the methods or compositions described may be varied or modified without departing from the spirit of the described aspects of the disclosure. We claim all such modifications and variations that fall within the scope and spirit of the claims below.

1. A method of treating a subject with a muscle wasting condition, the method comprising administering to the subject a composition comprising an effective amount of:
a) exogenous matrix bound nanovesicles (MBV) derived from extracellular matrix, wherein the MBV do not express CD63 and CD81 or are CD63^{lo}CD81^{lo} and wherein the MBV do not contain alkaline phosphatase;
b) myeloid progenitor cells or myeloid-derived cells treated with the exogenous MBV; and/or
c) conditioned media, or a fraction thereof, obtained from macrophages cultured in the presence of the exogenous MBV;
thereby treating the muscle wasting condition in the subject.

2-4. (canceled)

5. A method of promoting regeneration and repair of skeletal muscle in a subject in need thereof, the method comprising administering to the subject a composition comprising an effective amount of exogenous matrix bound nanovesicles (MBV) derived from extracellular matrix, wherein the MBV do not express CD63 and CD81 or are CD63^{lo}CD81^{lo} and wherein the MBV do not contain alkaline phosphatase, thereby promoting regeneration and repair of skeletal muscle in the subject.

6. The method of claim 1, wherein the exogenous MBV are contained within an extracellular-matrix (ECM) hydrogel or pregel prepared from extracellular matrix (ECM), and wherein the MBV contained within the hydrogel or pregel are administered to the subject.

7-12. (canceled)

13. The method of claim 11, wherein the ECM hydrogel or pregel is prepared from extracellular matrix of urinary bladder, small intestine, heart, dermis, liver, kidney, uterus, brain, blood vessel, lung, bone, muscle, pancreas, placenta, stomach, spleen, colon, adipose tissue, or esophagus.

14. (canceled)

15. The method of claim 6, wherein the MBV are present in the ECM hydrogel or pregel at a concentration of at least about 1×10⁵ to 1×10²⁰ particles/mL.

16. The method of claim 1, wherein the MBV are: a) derived from extracellular matrix of urinary bladder, small intestine, heart, dermis, liver, kidney, uterus, brain, blood vessel, lung, bone, muscle, pancreas, placenta, stomach, spleen, colon, adipose tissue, or esophagus; b) not derived from bone or cardiac ECM; or c derived from extracellular matrix of urinary bladder, small intestine, dermis, liver, kidney, uterus, brain, blood vessel, lung, muscle, pancreas, placenta, stomach, spleen, colon, adipose tissue, or esophagus.

17-19. (canceled)

20. The method of any one of claim 1, wherein the MBV are derived from extracellular matrix from a human, monkey, pig, cow, or sheep.

21. The method of claim 1 wherein the MBV are administered to the subject by systemic administration.

22. (canceled)

23. The method of claim 1, wherein the MBV are administered to the subject by local administration to a tissue or area of interest.

24. The method of claim 23, wherein the tissue or area of interest is a muscle, a nerve or a spinal cord.

25. The method of claim 1, where the myeloid-derived cells are macrophages, monocytes, or granulocytes.

26. The method of claim 1, wherein the myeloid progenitor cells or myeloid-derived cells are autologous to the subject.

27. (canceled)

28. The method of claim 1, wherein the myeloid progenitor cells or myeloid-derived cells are treated with exogenous MBV in a cell culture and are isolated from the cell culture after treatment with the exogenous MBV in order to be administered to the subject.

29. (canceled)

30. The method of claim 25, wherein the macrophages are autologous to the subject.

31. (canceled)

32. The method of claim 5, wherein the subject has a muscle wasting disease.

33. The method of claim 32, wherein the muscle wasting condition is a spinal muscular atrophy (SMA).

34. (canceled)

35. The method of claim 32, wherein the muscle wasting condition is a muscular dystrophy.

36-38. (canceled)

39. The method of claim 1, wherein the composition a) increases myotube formation in the subject; b) maintains neuronal progenitor cells in an undifferentiated state in the subject; c) increases the migratory capacity of endothelial cells in the subject; and/or d) increases the growth of muscle tissue in the subject.

40-42. (canceled)

43. The method of claim 1, wherein the subject is human.

44. The method of claim 5, wherein the subject experiences a) an increase in satellite cell activation and/or myoblast activity in a skeletal muscle in need of regeneration or repair after administration of the composition; b) an increase in muscle strength in a skeletal muscle in need of repair or regeneration after administration of the composition as compared to before administration; and/or c) an increase in muscle mass after administration of the composition as compared to before administration.

45-48. (canceled)
